(19) United States
(12) Patent Application Publication
(10) Pub. No.: US 2025/0387561 A1
(43) Pub. Date: Dec. 25, 2025

(54) TUBE CONNECTOR FOR AN INFUSION SET

(71) Applicant: Unomedical A/S, Lejre (DK)

(72) Inventors: Chih-Ho Chen, Taipei City (TW); Hung-Hsiang Chen, Lejre (DK); Steffen Gyrn, Lejre (DK)

(21) Appl. No.: 19/260,352

(22) Filed: Jul. 4, 2025

Related U.S. Application Data
(63) Continuation of application No. PCT/EP2024/050131, filed on Jan. 4, 2024.
(60) Provisional application No. 63/436,956, filed on Jan. 4, 2023.
(60) Provisional application No. 63/436,954, filed on Jan. 4, 2023.
(60) Provisional application No. 63/436,942, filed on Jan. 4, 2023.
(60) Provisional application No. 63/436,917, filed on Jan. 4, 2023.
(60) Provisional application No. 63/436,914, filed on Jan. 4, 2023.

(51) Int. Cl. A61M 5/142; A61M 39/10

(52) U.S. Cl. CPC A61M 5/14248; A61M 39/1011; A61M 39/105; A61M 2039/1072; A61M 2205/0216; A61M 2205/586; A61M 2210/04

(57) ABSTRACT

The present disclosure relates to a tube connector (402) for an infusion set, for example for delivering insulin from an insulin pump. The tube connector includes a connector body (404) adapted for connection with a tube (406). The tube connector also includes opposing first and second latch arms (412, 414) resiliently joined to the connector body at flexing points (420, 422). The tube connector also includes a first lever (424) attached to the first latch arm and a second lever (426) attached to the second latch arm. The first and second levers extend alongside the first and second latch arms, respectively, such that pressing the first and second levers towards each other deflects the first and second latch arms relative to the body portion about the flexing points.

The present invention relates to a tube connector for an infusion set, an infusion set comprising a tube connector, an infusion portion for an infusion set, and an infusion system. In examples, the infusion system is for intermittent or continuous subcutaneous delivery of a medication or a therapeutic fluid, such as insulin. In examples, the infusion set is for intermittent or continuous subcutaneous delivery of a medication or a therapeutic fluid, such as insulin.

### BACKGROUND

Infusion systems are generally known in the art for delivering a medication or a therapeutic fluid to a subcutaneous site in a patient. For example, infusion systems are known for delivering insulin to a patient from an insulin pump. The combination of an insulin pump and an infusion system can provide benefits for controlling dosing volumes, configuring dosing schedules, and providing continuous or intermittent delivery. Infusion systems generally comprise an infusion set having an infusion portion with a housing that is attached to a user. The infusion portion is typically adhered to a user's skin, and may remain in place for several days before being replaced. The infusion portion includes a tubular cannula extending from the housing, and the infusion portion is adapted to receive the medication via a connecting tube for suitable connection to further components of the infusion system, for example a pump. The infusion set includes a tube connector for connecting the tube to the infusion portion. The tube connector provides the possibility of disconnecting the infusion portion from the connecting tube and further parts of the infusion set and system in order to improve user comfort. Disconnecting the connecting tube may allow the user to perform activities that are hindered or prevented by the pump and other components.

It is common to make an infusion set (including the housing, the connecting tube, and the tube connector) small and compact so that the infusion system is not obstructive or uncomfortable for the user to wear for prolonged periods of time. However, the compact design of the infusion set may make it more difficult for some users to use the infusion set, for example to connect and disconnect the tube connector from the infusion portion. In particular, users with impaired movement and dexterity may have more difficulty using compact infusion sets. Such users may be elderly, and/or may suffer from specific conditions such as Parkinson's disease, Essential Tremor, other central nervous system (CNS) disorders, and/or arthritis.

### SUMMARY

According to the present invention, there is provided a tube connector for an infusion set, the tube connector including a connector body adapted for connection with a tube, opposing first and second latch arms resiliently joined to the connector body at flexing points, and a first lever attached to the first latch arm and a second lever attached to the second latch arm, the first and second levers extending alongside the first and second latch arms, respectively, such that pressing the first and second levers towards each other deflects the first and second latch arms relative to the connector body about the flexing points.

Pressing the first and second levers to deflect the first and second latch arms may cause the latch arms to disengage corresponding latches in the infusion set, for connecting and disconnecting the tube connector and the infusion set. The levers thereby reduce the force needed to connect and disconnect the tube connector and the infusion set, which may be advantageous for users with impaired movement and dexterity. In addition, providing separate levers, rather than relying on the user to directly contact the latch arms, means the levers can be larger and easier to grip, and longer to increase leverage, making the tube connector easier to use, particularly for users with impaired movement and dexterity.

In some examples, the connector body may include a tube connection point. The first latch arm and the first lever may be arranged on a first side of the tube connection point. The second latch arm and the second lever may be arranged on a second side of the tube connection point, opposite to the first side. The tube connection point may be centrally located between the first and second latch arms. In examples, the first latch arm is disposed between the first lever and the tube connection point, and the second latch arm may be disposed between the second lever and the tube connection point. In this way, the latch arms are disposed inwards of the levers. The levers may be the outermost parts of the tube connector, making them easy to identify and to grip.

In examples, the connector body may be substantially planar in a plane. The first and second latch arms and the first and second levers may be arranged in substantially the same plane as the plane of the connector body. That is, the tube connector, in particular the connector body, may be substantially flat and planar, and the latch arms and levers may be arranged in the same plane as the connector body. In examples, the tube connection point may also be substantially level with the plane of the connector body. In some examples, the first and second latch arms, first and second levers, and the tube connection point may be aligned in the plane of the connector body.

In examples, the first and second latch arms may each comprise a latch. The latch of the first latch arm may extend towards the first lever, and the latch of the second latch arm may extend towards the second lever. Each latch may comprise a protrusion, for example a hook-shaped protrusion. Each latch may have a shape corresponding to a corresponding latch part in the infusion set.

In examples, the first lever may be joined to the first latch arm along a portion of the first latch arm, and where the second lever is joined to the second latch arm along a portion of the second latch arm. The first lever may be joined to the first latch arm along a minor portion of the first latch arm, proximate the flexing point, and the second lever may be joined to the second latch arm along a minor portion of the second latch arm, proximal the flexing point. In examples, the first and second levers may extend further from the connector body than the first and second latch arms, respectively. Therefore, the first and second levers can be elongated beyond the ends of the first and second latch arms to increase the leverage when a user squeezes the levers.

In examples, the first and second levers may each comprise a gripping surface. The gripping surface may be a contoured surface, such as with notches, knurling, or other contours. The contours may improve gripping of the first and second levers by a user.

The tube connector may also include further includes at least one guide arm extending from the connector body. In examples, the at least one guide arm may be substantially parallel with the first and second latch arms. In examples, the at least one guide arm may be arranged in substantially the same plane as the first and second latch arms. In examples, the tube connector comprises two guide arms spaced apart and parallel to each other.

In examples, the tube connector may also include a front portion having a fluid connector for fluidly coupling to an infusion part of the infusion set, and a rear portion that includes a tube connection for the tube. The flexing points may arranged at the rear portion such that the first and second latch arms extend from the rear portion towards the front portion. In examples, the first and second latch arms and the first and second levers may extend beyond the front portion of the tube connector.

According to a further aspect of the present invention, there is also provided an infusion set that includes an infusion portion having a cannula and a mounting portion for mounting the infusion portion to a patient's skin with the cannula in a subcutaneous position. The infusion set also includes a tube connector as described above.

In examples, the infusion set may include at least one opening for the first and/or second latch arm of the tube connector. The opening may include a latch portion for engagement by the first and/or second latch arm. In examples, the infusion set may comprise a first opening for the first latch arm and a second opening for the second latch arm. Each of the first and second openings may comprise a latch portion for engagement by the first and second latch arms, respectively.

In examples, the infusion set may also include a guide opening for a guide arm of the tube connector.

In examples, the infusion set may also include a body portion, and when the tube connector is connected to the infusion part the first and second levers may be arranged alongside the body portion and spaced therefrom. In this way, the first and second levers of the tube connector may be easily gripped and squeezed for connecting and disconnecting the tube connector and the infusion set.

According to a further aspect of the present invention, there is also provided an infusion system that includes the infusion set described above, and a source of medicament for delivery to the patient via the tube connector and the cannula.

In examples, the infusion system may also comprise a pump for continuous or intermittent delivery of medicament to the patient.

According to a further aspect of the present invention, there is also provided an infusion set insertion device for attaching an infusion set to a patient, the infusion set insertion device includes an insertion device body and a button operable to move an infusion set within the insertion device body, and the button and/or the insertion device body includes an opening adapted to receive a safety connector such that the safety connector prevents movement of the button relative to the insertion device body. The safety connector includes a connector body, opposing first and second latch arms resiliently joined to the connector body at flexing points, and a first lever attached to the first latch arm and a second lever attached to the second latch arm, the first and second levers extending alongside the first and second latch arms, respectively, such that pressing the first and second levers towards each other deflects the first and second latch arms relative to the body portion about the flexing points.

It will be appreciated that the safety connector may be similar to the tube connector described above, but without any connection for the connecting tube. This allows the safety connector to be connected to the infusion set in place of the tube connector, for example to seal the infusion set when the tube connector is disconnected. In some examples, the tube connector may comprise a fluid path and a septum that allows a user to inject, e.g., using a syringe, medicament into the infusion set via the safety connector when it is connected to the infusion set.

Further aspects of the present invention relate to use of the tube connector, infusion set, and infusion system described above. In examples, the tube connector, infusion set, and infusion system described above may be used to subcutaneously deliver a liquid medicament to patient. Such use of the tube connector, infusion set, and infusion system may be a method of treatment.

In examples, the method of treatment may be a method of treating diabetes by delivering insulin into the body of a patient using the tube connector, infusion set, and infusion system described above. The insulin may be delivered in a continuous manner, or intermittently, for example in response to detected blood sugar or on a pre-determined delivery schedule.

In other examples, the method of treatment may be a method of treating a central nervous system disorder, for example Parkinson's disease, by delivering a medicament into the body of a patient using the tube connector, infusion set, and infusion system described above. The medicament may levodopa (optionally with carbidopa), a dopamine agonists (particularly Apomorphine), and/or a monoamine oxidase-B inhibitor.

The method of treatment may comprise attaching the infusion set to a patient's skin, for example with an adhesive patch, such that the cannula is subcutaneously positioned. Attaching the infusion set may comprise using an insertion device, and may include removing a safety connector from the insertion device before using the insertion device to attach the infusion set to the patient's skin. The method of treatment may further comprise connecting a medicament source to the infusion set by the tube connector described above. The medicament source may be a pump. The method of treatment may further comprise delivering a medicament to the patient via the connecting tube, tube connector, and infusion set.

According to a further aspect of the present invention, there is provided an infusion portion of an infusion set. The infusion portion comprises a cannula for subcutaneous delivery of a medicament when the infusion portion is attached to a patient's skin. The infusion portion also includes a spigot having a side wall with a regular polygonal cross-section with a number of sides exactly divisible by four. A plurality of fluid ports are disposed in the side wall of the spigot, and each of the plurality of fluid ports is connected with the cannula.

Advantageously, a tube connector can be connected to the infusion portion in a sideways sliding motion to connect to one of the fluid ports. The connection can be made in a plurality of different positions because there are a plurality of fluid ports. This may improve usability for a patient with impaired movement or dexterity by allowing them to find an angle and direction that best suits their ability. In addition, the polygonal cross-section of the spigot can help to create correct alignment between the tube connector and the fluid port by using opposing sides of the spigot as a guide for the tube connector.

In examples, the side wall may have at least four sides, for example four sides, or eight sides, or twelve sides, or sixteen sides. Each of the sides is the same length so that sides are arranged in opposing pairs.

In examples, each side of the side wall may comprise one of the plurality of fluid ports. That is, each side of the spigot may comprise a fluid port so that a tube connector can be connected from the direction of any of the sides.

In examples, each side may comprise a recess, and on each side the fluid port may be located in the recess. The recess may help a needle of the tube connector to align with the fluid port during connection.

In examples, the spigot may further comprise a flange edge overhanging the side wall. The flange edge may comprise a plurality of guides. Each of the plurality of guides may comprise a recess. Each recess may be aligned with a fluid port. A tube connector can cooperate with a guide/recess to ensure alignment with a fluid port during connection of the tube connector to the infusion portion.

In examples, the infusion portion may further comprise a base portion. The spigot may protrude from the base portion. The base portion may be attachable to a patient's skin, for example by an adhesive patch. In examples, the base portion may comprise a plurality of guides. Each of the plurality of guides may comprise a recess. Each recess may be aligned with a fluid port. A tube connector can cooperate with a guide/recess to ensure alignment with a fluid port during connection of the tube connector to the infusion portion.

In examples, the infusion portion may further comprise a cannular chamber in fluid communication with the cannular. Each of the plurality of fluid ports may be in communication with the cannular chamber.

In examples, each of the plurality of fluid ports comprises a seal, for example a septum seal. The tube connector may have a needle arranged to pierce the septum seal of a fluid port during connection of the tube connector to the infusion portion. The septum may re-seal on removal of the needle, allowing the cannula to be sealed when the tube connector is detached.

According to a further aspect of the present invention, there is provided a tube connector. The tube connector is for connecting to an infusion portion having a side wall with a regular polygonal cross section with a number of sides exactly divisible by four. The infusion portion also has a plurality of fluid ports. The tube connector comprises a first arm and a second arm spaced from and parallel to the first arm. The tube connector also comprises a needle extending between the first and second arms. The first arm is spaced from the second arm such that the first and second arms engage opposite sides of the side wall of the infusion portion when the tube connector is connected to the infusion portion to align the needle with a fluid port between the opposite sides.

Advantageously, the first and second arms create alignment between the needle and a fluid port as the tube connector is connected to the infusion portion in a sideways sliding movement.

In examples, the tube connector may further comprise a guide arranged to engage a corresponding guide of the infusion portion when the tube connector is connected to the infusion portion. In examples, the guide may comprise a protrusion. The protrusion may be arranged to engage a recess of the infusion portion when the tube connector is connected to the infusion portion. The recess on the infusion portion may be provided on a flange edge of the spigot, and/or on a base portion of the infusion portion.

In examples, the tube connector may further comprise a connecting tube and a fluid path connecting the connecting tube to the needle. The connecting tube may be detachable from the tube connector.

In examples, the tube connector may further comprise a rotatably mounted locking member. When the tube connector is connected to the infusion portion the locking member may be rotatable to engage the infusion portion and hold the tube connector to the infusion portion.

In examples, the locking member may comprise a loop member. When the tube connector is connected to the infusion portion the loop member may be rotatable to at least partly surround a part of the infusion portion, for example it may loop around an opposite side of the infusion portion, in particular the spigot. In examples, the locking member may be rotatably mounted to the tube connector about a rotational axis perpendicular to the needle. In examples, the locking member may comprise a catch configured to retain the locking member in the locked position.

In examples, the first arm of the tube connector may comprise a first latch and the second arm comprises a second latch. The first latch and the second latch may be arranged to clip onto the spigot of the infusion portion when the tube connector is connected to the infusion portion. In this way, the tube connector can be retained in the connected position, and the clipping may provide a tactile and/or audible indication that the tube connector has been properly connected to the infusion portion. In examples, the first latch and the second latch may be arranged to engage recesses on opposite sides of the spigot when the tube connector is connected to the infusion portion.

In examples, the tube connector may comprise a first loop member and a second loop member joined together via an opening. The second loop member may be sized such that the infusion portion can protrude through the second loop member. The first loop member may comprise the first arm, the second arm, and the needle. In this example, the tube connector can be connected to the infusion portion by positioning the infusion portion in the first loop member and the sliding the tube connector sideways so that the infusion portion passes through the opening and into the first loop member an dis engaged by the needle, the first arm, and the second arm.

According to a further aspect of the present invention there is also provided an infusion set comprising the infusion portion and the tube connector described above.

According to a further aspect of the present invention there is also provided an infusion system comprising the infusion set and a source of medicament. The source of medicament may be a syringe or a pump. The pump may be an insulin pump. The pump may provide continuous or intermittent delivery of medicament to the patient via the infusion set.

According to a further aspect of the present invention, there is also provided an infusion set insertion device for attaching an infusion set to a patient, the infusion set insertion device includes an insertion device body and a button operable to move an infusion set within the insertion device body, and the button and/or the insertion device body includes an opening adapted to receive a safety connector such that the safety connector prevents movement of the button relative to the insertion device body.

The safety connector may be similar to the tube connector described above, but without any connection for the connecting tube and without any needle. This allows the safety connector to be connected to the infusion set in place of the tube connector, for example to seal the infusion set when the tube connector is disconnected. In some examples, the tube connector may comprise a fluid path and a septum that allows a user to inject, e.g., using a syringe, medicament into the infusion set via the safety connector when it is connected to the infusion set.

Further aspects of the present invention relate to use of the infusion portion, tube connector, infusion set, and infusion system described above. In examples, the infusion portion, tube connector, infusion set, and infusion system described above may be used to subcutaneously deliver a liquid medicament to patient. Such use of the infusion portion, tube connector, infusion set, and infusion system may be a method of treatment.

In examples, the method of treatment may be a method of treating diabetes by delivering insulin into the body of a patient using the infusion portion, tube connector, infusion set, and infusion system described above. The insulin may be delivered in a continuous manner, or intermittently, for example in response to detected blood sugar or on a pre-determined delivery schedule.

In other examples, the method of treatment may be a method of treating a central nervous system disorder, for example Parkinson's disease, by delivering a medicament into the body of a patient using the infusion portion, tube connector, infusion set, and infusion system described above. The medicament may levodopa (optionally with carbidopa), a dopamine agonists (particularly Apomorphine), and/or a monoamine oxidase-B inhibitor.

The method of treatment may comprise attaching the infusion portion to a patient's skin, for example with an adhesive patch, such that the cannula is subcutaneously positioned. Attaching the infusion set may comprise using an insertion device, and may include removing a safety connector from the insertion device before using the insertion device to attach the infusion set to the patient's skin. The method of treatment may further comprise connecting a medicament source to the infusion set by the tube connector described above. The medicament source may be a pump. The method of treatment may further comprise delivering a medicament to the patient via the connecting tube, tube connector, and infusion portion.

According to a further aspect of the present invention, there is provided a tube connector for an infusion set, the tube connector comprising:
a body portion comprising a tube connection point for connection with a tube and a fluid connector for fluidly coupling to an infusion portion of the infusion set,
a first latch arm and a second latch arm extending from the body portion on opposite sides of the fluid connector, and
a first tab attached to the first latch arm and a second tab attached to the second latch arm,
wherein the first and second tabs are attached to the first and second latch arms, respectively, on sides opposite to the fluid connector and extend over the body portion and spaced from the body portion such that pressing the first tab and the second tab towards the body portion deflects the first and second latch arms, respectively.

During connection and disconnection the first tab and the second tab advantageously provide improved contact area for a user to grip, and improved leverage for deflecting the first latch arm and the second latch arm. In particular, the first tab and the second tab together define a pressing surface for a user to press on, in particular with a single thumb or finger. The user can therefore press down on the tube connector to depress the first tab and the second tab for connecting and disconnecting the tube connector and the infusion portion of the infusion set. This advantageously reduces the force required to deflect the first latch arm and the second latch arm, and makes the force easier to apply, which may be advantageous for users with impaired movement.

In examples, the first latch arm and the second latch may be are joined to the body portion at respective first and second flexing points. The first and second flexing points may be adapted to flex when the first tab and the second tab are pressed towards the body portion. In examples, the body portion may comprise a front side and a rear side. In examples, the tube connection point may be disposed on the rear side and the fluid connector may be disposed on the front side. In examples, the first and second flexing points may be arranged on the rear side.

In examples, the first latch arm may comprise a first latch and the second latch arm may comprise a second latch. The first and second latches may extend from the first and second latch arms in a direction towards the first and second tabs.

In examples, the first tab and the second tab may be spaced apart from each other proximate to a central part of the body portion. In examples, the fluid connector may be disposed in the central part of the body portion.

In examples, the fluid connector may comprise a needle. In examples, the first latch arm and the second latch arm may be parallel to the needle. In examples, the first latch arm and the second latch arm may be arranged in the same plane as the needle. In examples, the needle may engage a fluid connector of the infusion portion when the tube connector is connector to the infusion portion.

In other examples, the fluid connector may comprise a port, optionally with a seal, arranged to engage a needle of the infusion portion of the infusion set when the tube connector is connector to the infusion portion.

In examples, the first latch arm and the second latch arm may protrude beyond a front side of the body portion.

In examples, the tube connector may further comprise at least one guide arm extending from the body portion. In examples, the tube connector may comprise two guide arms. In examples, the at least one guide arm may extend parallel to the first latch arm and the second latch arm. In examples, the at least one guide arm may be disposed between of the first and second latch arms. In examples, the at least one guide arm is in the same plane as the first and second latch arms. In examples, the at least one latch arm does not deflect when the first and second tabs are pressed towards the body portion. In particular, the at least one guide arm may extend from the body portion inwardly (i.e., towards the central part of the body portion) of the flexing point(s). Accordingly, the first and second latch arms are outward of the at least one guide arm, and the at least one guide arm will not be deflected when the first and second tabs are pressed towards the body portion.

According to the present invention, there is also provided an infusion set comprising an infusion portion having a cannula and a mounting portion for mounting the infusion portion to a patient's skin with the cannula in a subcutaneous position, and a tube connector as described above.

In examples, the infusion set may comprise at least one opening for the first and/or second latch arm. In examples, the opening may comprise a latch portion for engagement by the first and/or second latch arm.

In examples, the infusion set may comprise a first opening for the first latch arm and a second opening for the second latch arm. In examples, each of the first and second openings may comprise a latch portion for engagement by the first and second latch arms, respectively.

In examples, the infusion portion may further comprise a guide opening for at least one guide arm of the tube connector.

According to the present invention, there is also provided an infusion system comprising the infusion set described above, and a source of medicament for delivery to the patient via the tube connector and the cannula.

In examples, the infusion system may further comprise a pump for continuous or intermittent delivery of medicament to the patient.

According to the present invention, there is also provided an infusion set insertion device for attaching an infusion set to a patient, the infusion set insertion device comprising an insertion device body and a button operable to move an infusion set within the insertion device body, and wherein the button and/or the insertion device body comprises an opening adapted to receive a safety connector such that the safety connector prevents movement of the button relative to the insertion device body, and wherein the safety connector comprises:
a body portion having a central part,
a first latch arm and a second latch arm extending from the body portion on opposite sides of the central part, and
a first tab attached to the first latch arm and a second tab attached to the second latch arm,
wherein the first and second tabs are attached to the first and second latch arms, respectively, on sides opposite to the central part and extend over the body portion and spaced from the body portion such that pressing the first tab and the second tab towards the body portion deflects the first and second latch arms, respectively.

It will be appreciated that the safety connector may be similar to the tube connector described above, but without any connection for the connecting tube. This allows the safety connector to be connected to the infusion set in place of the tube connector, for example to seal the infusion set when the tube connector is disconnected. In some examples, the tube connector may comprise a fluid path and a septum that allows a user to inject, e.g., using a syringe, medicament into the infusion set via the safety connector when it is connected to the infusion set.

Further aspects of the present invention relate to use of the tube connector, infusion set, and infusion system described above. In examples, the tube connector, infusion set, and infusion system described above may be used to subcutaneously deliver a liquid medicament to patient. Such use of the tube connector, infusion set, and infusion system may be a method of treatment.

In examples, the method of treatment may be a method of treating diabetes by delivering insulin into the body of a patient using the tube connector, infusion set, and infusion system described above. The insulin may be delivered in a continuous manner, or intermittently, for example in response to detected blood sugar or on a pre-determined delivery schedule.

In other examples, the method of treatment may be a method of treating a central nervous system disorder, for example Parkinson's disease, by delivering a medicament into the body of a patient using the tube connector, infusion set, and infusion system described above. The medicament may levodopa (optionally with carbidopa), a dopamine agonists (particularly Apomorphine), and/or a monoamine oxidase-B inhibitor.

The method of treatment may comprise attaching the infusion set to a patient's skin, for example with an adhesive patch, such that the cannula is subcutaneously positioned. Attaching the infusion set may comprise using an insertion device, and may include removing a safety connector from the insertion device before using the insertion device to attach the infusion set to the patient's skin. The method of treatment may further comprise connecting a medicament source to the infusion set by the tube connector described above. The medicament source may be a pump. The method of treatment may further comprise delivering a medicament to the patient via the connecting tube, tube connector, and infusion set.

According to a further aspect of the present invention, there is provided a tube connector for an infusion set. The tube connector comprises a connector body having a tube connection point for connection with a tube and a fluid path for connecting the tube with the infusion set during use. The tube connector further comprises an arm arranged to engage the infusion set when the tube connector is connected to the infusion set. The tube connector further comprises a loop portion extending from the connector body such that a user can pull the loop portion to disconnect the tube connector from the infusion set.

Advantageously, the loop portion may be gripped in a number of different ways, including as a pull ring, which may make it easier for a patient with impaired dexterity to grip and pull the tube connector to disconnect it from the infusion portion.

In examples, the tube connector comprises a first arm (the arm) and a second arm. The second arm may be parallel to and spaced from the first arm. The first arm and the second arm may each comprise a latch for connecting the tube connector to the infusion set. The latches of the first arm and the second arm may be facing each other. The arm, the first arm and/or the second arm may be resiliently deflectable.

In examples, the tube connector may further comprise a needle extending from the connector body and connected to the fluid path. The needle may be arranged to engage a fluid port of the infusion set when the tube connector is connected to the infusion set. The fluid port may include a seal, for example a septum, that is pierced by the needle. The needle may be parallel with the latch arm.

In examples, the loop portion may be rotatably connected to the connector body. The arm (or the first and second arms) may extend from the connector body in an axial direction, and the loop portion may be rotatably connected to the connector body about a rotational axis perpendicular to the axial direction. In examples, the loop portion may be joined to the connector body at a hinge.

In examples, when the tube connector is connected to the infusion set, the loop portion may be rotatable to engage the infusion set to retain the tube connector on the infusion set. That is, the loop portion may be a locking member. The loop portion may be configured to clip over a part of the infusion set.

In some examples, the infusion set comprises a spigot having a side wall with a fluid port. The side wall of the infusion set may comprise a plurality of fluid ports. The tube connector may be configured to be connected to the infusion set at any one of the plurality of fluid ports.

In examples, the infusion set comprises a guide corresponding to each fluid port. The tube connector may comprise a corresponding guide arranged to engage the guide of the infusion set to align with one of the plurality of fluid ports as the tube connector is connected to the infusion set.

According to a further aspect of the present invention there is also provided an infusion set comprising an infusion portion having a cannula and a mounting portion for mounting the infusion portion to a patient's skin with the cannula in a subcutaneous position, and a tube connector as described above.

In examples, the infusion portion may comprise a spigot having side wall with a fluid port. The side wall of the spigot may comprise a plurality of fluid ports. In examples, the spigot may comprise a regular polygonal cross-sectional shape having a number of sides exactly divisible by four. The tube connector may have a first arm and a second arm that engage opposite sides of the side wall of the infusion portion to ensure alignment when the tube connector is connected to the infusion portion.

In examples, the spigot may comprises one or more recesses. The arm (or the first arm and the second arm) of the tube connector may (each) comprise a latch arranged to engage one of the recesses of the spigot when the tube connected is connected to the infusion portion. In examples, one of the tube connector and the infusion portion comprises a needle.

The other of the tube connector and the infusion portion may comprise a fluid port. The fluid port may comprise a seal, for example a septum, that is pierceable by the needle.

In examples, the infusion portion may comprise a guide feature. The guide feature may be engaged by a corresponding guide feature of the tube connector to create alignment during connection of the tube connector to the infusion portion.

According to a further aspect of the invention, there is also provided an infusion system comprising the infusion set described above, and a source of medicament for delivery to the patient via the tube connector and the cannula.

In examples, the infusion system may further comprise a pump for continuous or intermittent delivery of medicament to the patient.

According to a further aspect of the invention, there is also provided an infusion set insertion device for attaching an infusion set to a patient. The infusion set insertion device comprises an insertion device body and a button operable to move an infusion set within the insertion device body. The button and/or the insertion device body comprises a connector opening adapted to receive a safety connector such that the safety connector prevents movement of the button relative to the insertion device body. The safety connector comprises an arm for engaging the infusion set, and a loop portion extending from the connector body such that a user can pull the loop portion to disconnect the tube connector from the infusion set.

According to a further aspect of the invention, there is also provided a tube connector for an infusion set, the tube connector comprising a connector body having a tube connection point for connection with a tube and a fluid path for connecting the tube with the infusion set during use. The tube connector further comprises an arm arranged to engage the infusion set when the tube connector is connected to the infusion set. The tube connector further comprises a locking member rotatably attached to the connector body and being rotatable to engage the infusion portion when the tube connector is connected to the infusion portion to retain the tube connector on the infusion set.

In examples, the locking member may be shaped to at least partly surround a part of the infusion set when in the locked position. The locking member may comprise a loop portion. The loop portion may surround a part of the infusion set, for example a spigot of the infusion set.

It will be appreciated that the safety connector may be similar to the tube connector described above, but without any connection for the connecting tube or a needle. This allows the safety connector to be connected to the infusion set in place of the tube connector, for example to seal the infusion set when the tube connector is disconnected. In some examples, the tube connector may comprise a fluid path and a seal (e.g., a septum) that allows a user to inject, e.g., using a syringe, medicament into the infusion set via the safety connector when it is connected to the infusion set.

Further aspects of the present invention relate to use of the tube connector, infusion set, and infusion system described above. In examples, the tube connector, infusion set, and infusion system described above may be used to subcutaneously deliver a liquid medicament to patient. Such use of the tube connector, infusion set, and infusion system may be a method of treatment.

In examples, the method of treatment may be a method of treating diabetes by delivering insulin into the body of a patient using the tube connector, infusion set, and infusion system described above. The insulin may be delivered in a continuous manner, or intermittently, for example in response to detected blood sugar or on a pre-determined delivery schedule.

In other examples, the method of treatment may be a method of treating a central nervous system disorder, for example Parkinson's disease, by delivering a medicament into the body of a patient using the tube connector, infusion set, and infusion system described above. The medicament may levodopa (optionally with carbidopa), a dopamine agonists (particularly Apomorphine), and/or a monoamine oxidase-B inhibitor.

The method of treatment may comprise attaching the infusion set to a patient's skin, for example with an adhesive patch, such that the cannula is subcutaneously positioned. Attaching the infusion set may comprise using an insertion device, and may include removing a safety connector from the insertion device before using the insertion device to attach the infusion set to the patient's skin. The method of treatment may further comprise connecting a medicament source to the infusion set by the tube connector described above. The medicament source may be a pump. The method of treatment may further comprise delivering a medicament to the patient via the connecting tube, tube connector, and infusion set.

According to a further aspect of the present invention, there is provided a tube connector for connecting to an infusion portion of an infusion set, the tube connector comprising:
a first loop portion and a second loop portion that together define a closed loop,
wherein the first loop portion is larger than the second loop portion and sized such that the first loop portion can be positioned over a spigot of the infusion portion,
wherein the second loop portion is sized to receive the spigot when the tube connector is moved sideways and the spigot of the infusion portion moves from the first loop portion into the second loop portion, and
wherein the tube connector further comprises a fluid connection disposed in the second loop portion for forming a fluid connection between the tube connector and the infusion portion.

Advantageously, the sideways sliding movement to connect the tube connector to the infusion portion may be easy for a patient with impaired dexterity.

In examples, the second loop portion may comprise a resiliently deflectable arm arranged to engage the spigot of the infusion portion. In examples, the second loop portion may comprise a first resiliently deflectable arm and an opposing second resiliently deflectable arm. The first and second resiliently deflectable arms may be arranged to engage opposite sides of the spigot of the infusion portion. Engagement between the or each resiliently deflectable arm and the spigot may create alignment between the tube connector and the spigot, in particular between the fluid connection of the tube connector and a corresponding feature of the infusion portion.

In examples, the or each resiliently deflectable arm may comprise a latch arranged to latch onto the spigot of the infusion portion. The or each latch may engage a recess on the spigot to clip the tube connector to the infusion portion.

In examples, the fluid connection of the tube connector may comprise a needle protruding into the second loop portion. During connection of the tube connector to the infusion portion the needle may engage a seal, for example a septum, of the infusion portion to create a fluid connection between the tube connector and the infusion portion. In other examples, the fluid connection of the tube connector may comprise a seal, for example a septum, and the infusion portion may comprise a needle arranged to engage the fluid connection.

In examples, the spigot of the infusion portion may comprise a polygonal cross-section having opposing parallel sides. In such examples, the tube connector may further comprise an opening between the first loop portion and the second loop portion. The opening may be sized such that the spigot can only pass through the opening when the direction of movement is parallel to the opposing parallel sides of the spigot. That is, a width of the opening may correspond to (e.g., be slightly larger than) a distance between opposing sides of the spigot so that the spigot can only pass through the opening in a correct alignment. In examples, the spigot of the infusion portion may comprise a regular polygonal cross-section having a number of sides that is exactly divisible by four. In this way, when two opposite parallel sides are aligned with the opening, a third side is perpendicular to the two opposite parallel sides and a fluid port of the third side can be engaged by the fluid connection (e.g., needle).

In examples, the tube connector may further comprise at least one protrusion extending into the closed loop. The opening may be defined between the at least one protrusion and an opposite side of the tube connector. In examples, the tube connector comprises a first protrusion on a first side of the closed loop and a second protrusion on an opposite side of the closed loop, and the opening is defined between the first and second protrusions. The or each protrusion may be tapered to guide the spigot through the opening.

In examples, the second loop portion may be shaped to match a cross-sectional shape of the spigot of the infusion portion. In some examples the spigot may have a generally triangular or other (regular) polygonal cross-sectional shape, and the second loop portion may comprise a corresponding profile that allows the spigot to slide into the second loop portion while creating an alignment between the fluid connection and a corresponding feature of the infusion portion (e.g., a fluid port).

In examples, the tube connector may further comprise a connecting tube in fluid communication with the fluid connection. The connecting tube may be for connection to a medicament source, for example a pump.

In examples, a part of the first loop portion opposite to the second loop portion is shaped to match a portion of the spigot. In this way, the profile of first loop portion can create an alignment between the fluid connection and a corresponding feature of the infusion portion (e.g., a fluid port) before the spigot moves into the second loop portion.

According to a further aspect of the invention there is also provided an infusion portion for an infusion set. The infusion portion comprises a base portion for attachment to a patient's skin and a cannula for subcutaneously delivering a medicament during use. The infusion portion comprises a spigot extending from the base portion and the spigot comprises a fluid port for fluid connection with the tube connector described above.

In examples, the spigot may comprise a polygonal cross-sectional shape having a plurality of sides. For example, the spigot may comprise a generally triangular cross-sectional shape with the fluid port arranged at one of the tips of the spigot. In such an example, the second loop portion of the tube connector may comprise a partial triangular profile to receive the spigot and align the fluid port and the fluid connection.

In other examples, the spigot may comprise a regular polygonal cross-sectional shape with the fluid port being arranged on one side of the spigot. In such an example the second loop portion of the tube connector may comprise a partial regular polygonal profile to receive the spigot and align the fluid port and the fluid connection.

In examples, the spigot may comprise a plurality of fluid ports disposed in different sides of the spigot. In this way, the tube connector may be connected to the infusion portion at a plurality of different angles.

In examples, each side of the spigot comprises a recess. One or more recesses may be engaged by one or more resiliently deformable arms of the tube connector to clip the tube connector to the infusion portion. Additionally or alternatively, at least one of the recesses may be engaged by a protrusion on the tube connector to align the fluid connection with a fluid port of the infusion portion.

In examples, the spigot may comprise a flange edge overhanging a side wall of the spigot opposite to the base portion. When the spigot is received in the second loop portion of the tube connector the second loop portion may be disposed between the base portion and the flange edge. This can help form a connection between the tube connector and the infusion portion.

According to a further aspect of the invention there is also provided an infusion set comprising the tube connector and the infusion portion described above.

According to a further aspect of the invention there is also provided an infusion system comprising the infusion set and a source of medicament, for example a medicament pump such as an insulin pump. The source of medicament may be a syringe or a pump. The pump may be an insulin pump. The pump may provide continuous or intermittent delivery of medicament to the patient via the infusion set.

According to a further aspect of the present invention, there is also provided an infusion set insertion device for attaching an infusion set to a patient, the infusion set insertion device includes an insertion device body and a button operable to move an infusion set within the insertion device body, and the button and/or the insertion device body includes an opening adapted to receive a safety connector such that the safety connector prevents movement of the button relative to the insertion device body.

The safety connector may be similar to the tube connector described above, but without any connection for the connecting tube and without any needle. This allows the safety connector to be connected to the infusion set in place of the tube connector, for example to seal the infusion set when the tube connector is disconnected. In some examples, the tube connector may comprise a fluid path and a septum that allows a user to inject, e.g., using a syringe, medicament into the infusion set via the safety connector when it is connected to the infusion set.

Further aspects of the present invention relate to use of the tube connector, infusion portion, infusion set, and infusion system described above. In examples, the tube connector, infusion portion, infusion set, and infusion system described above may be used to subcutaneously deliver a liquid medicament to patient. Such use of the tube connector, infusion portion, infusion set, and infusion system may be a method of treatment.

In examples, the method of treatment may be a method of treating diabetes by delivering insulin into the body of a patient using the tube connector, infusion portion, infusion set, and infusion system described above. The insulin may be delivered in a continuous manner, or intermittently, for example in response to detected blood sugar or on a pre-determined delivery schedule.

In other examples, the method of treatment may be a method of treating a central nervous system disorder, for example Parkinson's disease, by delivering a medicament into the body of a patient using the tube connector, infusion portion, infusion set, and infusion system described above. The medicament may levodopa (optionally with carbidopa), a dopamine agonists (particularly Apomorphine), and/or a monoamine oxidase-B inhibitor.

The method of treatment may comprise attaching the infusion portion to a patient's skin, for example with an adhesive patch, such that the cannula is subcutaneously positioned. Attaching the infusion set may comprise using an insertion device, and may include removing a safety connector from the insertion device before using the insertion device to attach the infusion set to the patient's skin. The method of treatment may further comprise connecting a medicament source to the infusion set by the tube connector described above. The medicament source may be a pump. The method of treatment may further comprise delivering a medicament to the patient via the connecting tube, tube connector, and infusion portion.

Other technical features may be readily apparent to one skilled in the art from the following figures, descriptions, and claims.

### BRIEF DESCRIPTION OF THE DRAWINGS

Examples of the invention are described with reference to the accompanying drawings, in which:

FIG. 1A illustrates attachment of an infusion portion of an infusion set to a user;

FIG. 1B illustrates a pump, connecting tube and tube connector of the infusion system;

FIG. 1C illustrates connection of the connecting tube to the infusion portion;

FIG. 2 illustrates an example infusion set having an infusion portion and a tube connector;

FIG. 3A illustrates a first example infusion portion of the infusion set of FIG. 2;

FIG. 3B illustrates a second example infusion portion of the infusion set of FIG. 2;

FIG. 4 illustrates the tube connector of the infusion set of FIG. 2;

FIG. 5A illustrates an infusion portion attachment tool and a safety connector;

FIG. 5B illustrates the safety connector of FIG. 5A;

FIG. 6A illustrates an example infusion portion;

FIG. 6B illustrates a cross-section of the infusion portion of FIG. 6A;

FIG. 7 illustrates a tube connector for the infusion portion of FIG. 6A and FIG. 6B;

FIG. 8A illustrates the tube connector of FIG. 7 and the infusion portion of FIG. 6A;

FIG. 8B illustrates another view of the tube connector of FIG. 7 and the infusion portion of FIG. 6A;

FIG. 8C illustrates connection of the tube connector of FIG. 7 to the infusion portion of FIG. 6A;

FIG. 8D illustrates the tube connector of FIG. 7 when connected to the infusion portion of FIG. 6A;

FIG. 9A illustrates a further example infusion portion;

FIG. 9B illustrates a tube connector being connected with the infusion portion of FIG. 9A;

FIG. 9C illustrates the tube connector and infusion portion once the tube connector has been connected and locked;

FIG. 10A illustrates a further example tube connector;

FIG. 10B illustrates initial connection of the tube connector for FIG. 10A to an infusion portion;

FIG. 10C illustrates the tube connector for FIG. 10A connected to an infusion portion;

FIG. 11A illustrates an example infusion portion insertion device and a safety connector;

FIG. 11B illustrates the infusion portion insertion device of FIG. 11A with the safety connector in a different configuration;

FIG. 11C illustrates a further example safety connector and an infusion portion insertion device;

FIG. 12 illustrates an example infusion set having an infusion portion and a tube connector;

FIG. 13A illustrates a first example infusion portion of the infusion set of FIG. 12;

FIG. 13B illustrates a second example infusion portion of the infusion set of FIG. 12;

FIG. 14A shows a top view of the tube connector of the infusion set of FIG. 12;

FIG. 14B shows a front view of the tube connector of the infusion set of FIG. 12;

FIG. 14C shows a rear view of the tube connector of the infusion set of FIG. 12;

FIG. 14D shows a different rear view of the tube connector of the infusion set of FIG. 12;

FIG. 15A illustrates an infusion portion attachment tool and a safety connector;

FIG. 15B illustrates the safety connector of FIG. 15A;

FIG. 16A illustrates an example infusion set having an infusion portion and a tube connector;

FIG. 16B illustrates a further view of the infusion set of FIG. 16A, with the tube connected locked onto the infusion portion;

FIG. 17 illustrates a first example infusion portion of the infusion set of FIG. 16A;

FIG. 18 illustrates the tube connector of the infusion set of FIG. 16A;

FIG. 19A illustrates a further example infusion portion of an infusion set;

FIG. 19B illustrates a cross-section of the infusion portion of FIG. 19A;

FIG. 20A illustrates an infusion set with the tube connector of FIG. 18 and the infusion portion of FIG. 19A and FIG. 19B;

FIG. 20B illustrates a further view of the infusion set of FIG. 20A;

FIG. 20C illustrates initial connection of the tube connector to the infusion portion of the infusion set of FIG. 20A and FIG. 20B;

FIG. 20D illustrates the infusion set of FIG. 20A to FIG. 20C with the tube connector connected to the infusion portion;

FIG. 21A illustrates a further example infusion portion of an infusion set;

FIG. 21B illustrates an infusion set with a tube connector and the infusion portion of FIG. 21A;

FIG. 21C illustrates the tube connector connected to the infusion portion of FIG. 21A;

FIG. 22A illustrates an infusion portion insertion device with a safety connector;

FIG. 22B illustrates a further view of the infusion portion insertion device of FIG. 22A;

FIG. 23 illustrates an example infusion portion of infusion set;

FIG. 24A illustrates an example tube connector for connecting to the infusion portion of FIG. 23;

FIG. 24B illustrates the tube connector of FIG. 24A;

FIG. 25A illustrates initial connection of the tube connector or FIG. 24A and FIG. 24B with the infusion portion of FIG. 23;

FIG. 25B illustrates the tube connector or FIG. 24A and FIG. 24B connected to the infusion portion of FIG. 23;

FIG. 26A illustrates an alternative example infusion portion of the infusion set;

FIG. 26B illustrates a cross-section of the infusion portion of FIG. 26A;

FIG. 27 illustrates a further example infusion portion of the infusion set;

FIG. 28 illustrates a tube connector for the infusion portions of FIG. 26A, FIG. 26B and FIG. 27;

FIG. 29A illustrates initial connection of the tube connector of FIG. 28 to the infusion portion of FIG. 26A, FIG. 26B or FIG. 27;

FIG. 29B illustrates the tube connector of FIG. 28 connected to the infusion portion; and

FIG. 30 illustrates an example safety connector and an infusion portion insertion device.

### DETAILED DESCRIPTION

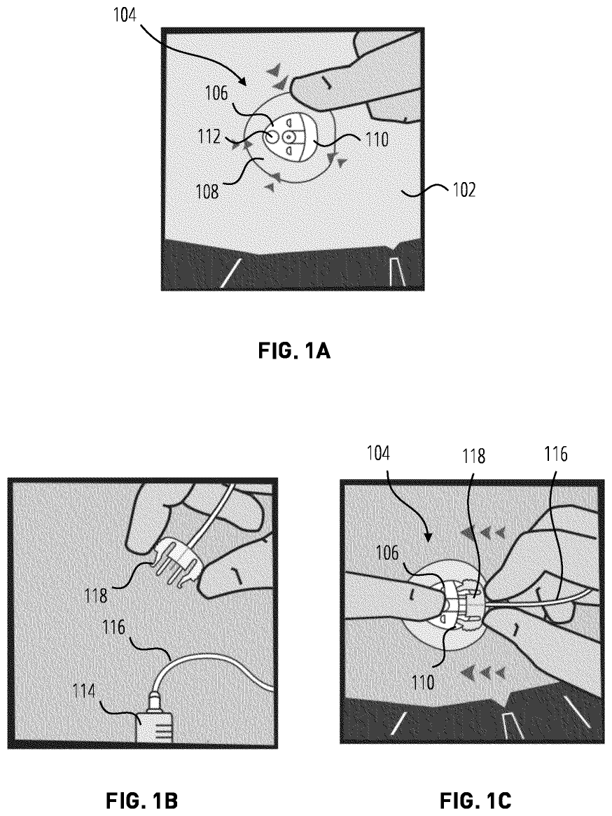

As shown in FIG. 1A, a user attaches an infusion portion 104 to their skin 102. The infusion portion 104 includes a housing 106 and a mounting portion 108 that attaches the housing 106 to the skin 102. In this example the mounting portion 108 is an adhesive patch that is adhered to the skin 102 to attach the infusion portion 104 to the user. The housing 106 also includes a cannula 112 that is subcutaneously positioned when the infusion portion 104 is attached to the skin 102. A tube connecting portion 110 is provided for connection of a connecting tube (see FIG. 1B and FIG. 1C below). The tube connecting portion 110 is fluidly connected to the cannula 112 such that medicament can flow from the tube connecting portion 110 into the cannula 112 for delivery to the user.

FIG. 1B illustrates a pump 114, connecting tube 116 and tube connector 118 that is connectable to the tube connecting portion 110 illustrated in FIG. 1A. The connecting tube 116 is connected between the pump 114 and the tube connector 118.

As shown in FIG. 1C, the tube connector 118 is connectable to the tube connecting portion 110 of the housing 106 when the infusion portion 104 is attached to the user. In this way, fluid can be pumped by the pump 114 (see FIG. 1B) through the connecting tube 116, the tube connector 118, the housing 106 and to the cannula 112 (see FIG. 1A) for delivery to the patient. The pump 114 (see FIG. 1B) can operate continuously or intermittently to deliver medicament at the desired rate.

The tube connector 118 can be disconnected from the tube connecting portion 110 of the housing 106, allowing the pump 114 to be disconnected but leaving the infusion portion 104 in position. Accordingly, the pump 114 can be removed and then re-attached by the tube connector 118.

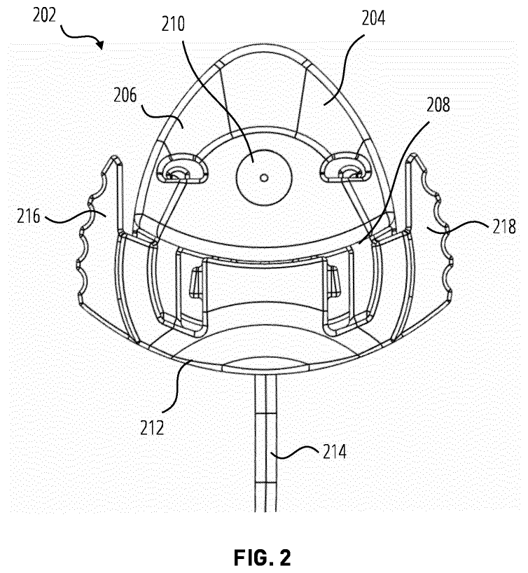

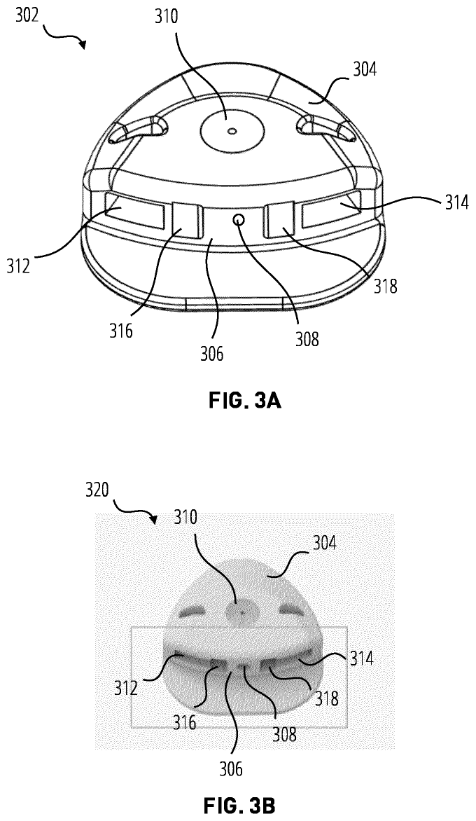

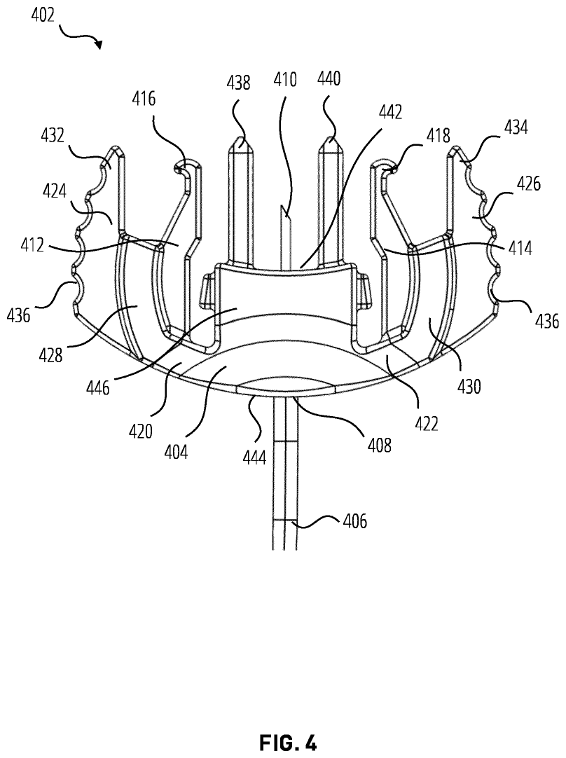

FIG. 2 illustrates an example infusion set 202. The infusion set 202 includes an infusion portion 204. The infusion portion 204 has a body portion 206 that is attachable to a user as described with reference to FIG. 1A to FIG. 1C. A cannula 210 is provided within the body portion 206 and the cannula 210 is subcutaneously positioned when the body portion 206 is attached to the user. It will be appreciated that the cannula 210 protrudes from the opposite side of the body portion 206 to that illustrated, and the top of cannula 210 is indicated in FIG. 2. The infusion portion 204 also includes a tube connecting portion 208 for connecting with a connecting tube 214 via a tube connector 212. The tube connecting portion 208 and tube connector 212 are described in more detail with reference to FIG. 3A to FIG. 4.

As shown, the tube connector 212 includes a first lever 216 and a second lever 218. As described further below, from the position shown in FIG. 2 the first lever 216 and the second lever 218 can be pushed inwards, towards the body portion 206, to disconnect tube connector 212 from the infusion portion 204. As shown, when the tube connector 212 is connected to the infusion portion 204 the first lever 216 and the second lever 218 extend either side of the body portion 206 and are spaced from the body portion 206.

FIG. 3A illustrates a first example infusion portion 302 that may be the infusion portion 204 described with reference to FIG. 2. As shown, the infusion portion 302 includes a body portion 304 that is attachable to a user as described with reference to FIG. 1A to FIG. 1C. A cannula 310 is provided within the body portion 304 and the cannula 310 is subcutaneously positioned when the body portion 304 is attached to the user. It will be appreciated that the cannula 310 protrudes from the opposite side of the body portion 304 to that illustrated, and the top of cannula 310 is indicated in FIG. 2. The infusion portion 302 also includes a tube connecting portion 306 for connecting with a connecting tube via a tube connector as described further with reference to FIG. 4.

The tube connecting portion 306 includes a fluid connector 308 for forming a fluid connection with the tube connector and connecting tube shown in FIG. 4. The fluid connector 308 may include a septum or other seal that is pierced by the tube connector, and allows for resealing the fluid connector 308 when the tube connector is disconnected from the infusion portion 302.

As shown in FIG. 3A, the tube connecting portion 306 includes a first latch opening 312 and a second latch opening 314 that receive first and second latch arms of the tube connector described further with reference to FIG. 4. The first latch opening 312 and the second latch opening 314 include internal latch portions as described further below. The tube connecting portion 306 also includes a first guide opening 316 and a second guide opening 318. The first guide opening 316 and the second guide opening 318 are arranged to receive first and second guide arms of the tube connector described further with reference to FIG. 4.

FIG. 3B illustrates a further example infusion portion 320 that may be the infusion portion 204 described with reference to FIG. 2. The infusion portion 320 of FIG. 3B is similar to that of FIG. 3A and the same reference numerals are used for alike features. The difference between the infusion portion 302 of FIG. 3A and the infusion portion 320 of FIG. 3B is that the first latch opening 312 and the first guide opening 316 are formed as separate parts of a single opening in the body portion 304. Similarly, the second latch opening 314 and the second guide opening 318 are formed as separate parts of a single opening in the body portion 304. In particular, the first latch opening 312 is formed in a first part of the opening arranged generally outwardly from the fluid connector 308, and the first guide opening 316 is arranged in a second part of the opening arranged adjacent to the fluid connector 308. In the same manner, the second latch opening 314 is formed in a first part of the opening arranged generally outwardly from the fluid connector 308, and the second guide opening 318 is arranged in a second part of the opening arranged adjacent to the fluid connector 308.

FIG. 4 illustrates a tube connector 402 for use with the infusion portion 302 of FIG. 3A and the infusion portion 320 of FIG. 3B.

As shown in FIG. 4, the tube connector 402 includes a connector body 404. The connector body 404 includes a front side 442 and a rear side 444. The connector body 404 has is symmetrical about a mid-plane.

The connecting tube 406 is connected to a fluid path 408 at a first side of the connector body 404, and a needle 410 extends from an opposite side of the connector body 404. The connecting tube 406, fluid path 408 and needle 410 are arranged on the mid-plane of the connector body 404. The connecting tube 406 is connected to the fluid path 408 at the rear side 444 and the needle 410 is connected to the fluid path 408 at the front side 442 and protrudes beyond the front side 442. When the tube connector 402 is connected to the infusion portion 302 (FIG. 3A) or infusion portion 320 (FIG. 3B) the needle 410 engages the fluid connector 308 to form a fluid connection between the connecting tube 406 and the cannula 310.

As shown in FIG. 4, the tube connector 402 includes a first latch arm 412 and a second latch arm 414. The first latch arm 412 and the second latch arm 414 extend from the connector body 404 on the front side 442. The first latch arm 412 and the second latch arm 414 protrude from the connector body 404. The first latch arm 412 and the second latch arm 414 are substantially parallel to each other. The first latch arm 412 includes a first latch 416 at a distal end of the first latch arm 412 (the end opposite to the connector body 404). The second latch arm 414 includes a second latch 418 at a distal end of the second latch arm 414 (the end opposite to the connector body 404). The first latch 416 is directed away from the second latch arm 414 and the second latch 418 is directed away from the first latch arm 412. That is, the first latch 416 and the second latch 418 are directed outwardly, and away from the mid-plane of the tube connector 402. As described below, the first latch 416 and the second latch 418 are configured to latch onto internal latch points of the first latch opening 312 and the second latch opening 314 of the infusion portion 302, 320, respectively, during use.

The first latch arm 412 is joined to the connector body 404 at a first flexing point 420. The second latch arm 414 is joined to the connector body 404 at a second flexing point 422. The first flexing point 420 and the second flexing point 422 are resiliently deformable such that the first latch arm 412 and the second latch arm 414 are resiliently deflectable, allowing the first latch 416 and the second latch 418 to be latched and unlatched as described below. The first flexing point 420 and the second flexing point 422 may be formed by a thinner section of the connector body 404, or by a different material (e.g., provided by co-moulding), or by an embedded or attached feature such as a spring feature. As shown in FIG. 4, the first latch arm 412 and the second latch arm 414 are both spaced from a central part 446 of the connector body 404, and the spacing allows the first latch arm 412 and the second latch arm 414 to be deflected inwardly.

As shown in FIG. 4, a first lever 424 is joined to the first latch arm 412. Similarly a second lever 426 is joined to the second latch arm 414. The first lever 424 and the second lever 426 are positioned outwardly of the mid-plane of the connector body 404. The first lever 424 and the second lever 426 are substantially parallel to the first latch arm 412 and the second latch arm 414 and extend alongside the first latch arm 412 and the second latch arm 414, respectively. The first lever 424 and the second lever 426 can be pressed inwards, towards each other, to deflect the first latch arm 412 and the second latch arm 414 for connecting and disconnecting the tube connector 402 from the infusion portion 302, 320.

As shown in FIG. 4, the first lever 424 is joined to the first latch arm 412 along a part of the length of the first lever 424 and along a part of the length of the first latch arm 412. The first lever 424 is joined to the first latch arm 412 at joined portion 428, and the first latch arm 412 protrudes beyond the joined portion 428. A free portion 432 of the first lever 424 also extends beyond the joined portion 428. Similarly, the second lever 426 is joined to the second latch arm 414 along a part of the length of the second lever 426 and along a part of the length of the second latch arm 414. The second lever 426 is joined to the second latch arm 414 at joined portion 430, and the second latch arm 414 protrudes beyond the joined portion 430. A free portion 434 of the second lever 426 also extends beyond the joined portion 430. The distal ends of the first lever 424 and the second lever 426 are substantially level with the distal ends of the first latch arm 412 and the second latch arm 414, and in some examples the first lever 424 and the second lever 426 may extend further than the first latch arm 412 and the second latch arm 414. As shown, each of the first lever 424 and the second lever 426 includes a gripping surface 436.

As shown in FIG. 2, when the tube connector 212, 402 is connected to the infusion portion 204 the first lever 216, 424 and the second lever 218, 426 extend along opposite sides of the infusion portion 204.

Referring again to FIG. 4, the tube connector 402 also includes a first guide arm 438 and a second guide arm 440. The first guide arm 438 and the second guide arm 440 protrude from the connector body 404, in particular from the central part 446, in a forwards direction. That is, the first guide arm 438 and the second guide arm 440 extend from the front side 442 of the connector body 404. The first guide arm 438 is adapted to be received in the first guide opening 316 of the infusion portion 302, 320 (see FIG. 3A and FIG. 3B), and the second guide arm 440 is adapted to be received in the second guide opening 318 of the infusion portion 302, 320 (see FIG. 3A and FIG. 3B). The first guide arm 438 and the second guide arm 440, in cooperation with the first guide opening 316 and the second guide opening 318 respectively, act to guide connection of the tube connector 402 and the infusion portion 302, 320 and particularly to align the needle 410 with the fluid connector 308.

Referring to FIG. 3A, FIG. 3B and FIG. 4, during connection of the tube connector 402 to the infusion portion 302, 320 the first latch arm 412 is received in the first latch opening 312 and the second latch arm 414 is received in the second latch opening 314. The first guide arm 438 and the second guide arm 440 are simultaneously received in the first guide opening 316 and the second guide opening 318, respectively. As the tube connector 402 is pushed onto the infusion portion 302, 320 the first latch 416 and the second latch 418 will engage corresponding latch features in the first latch opening 312 and the second latch opening 314, respectively. Engagement of the first guide opening 316 and the second guide opening 318 with the latch features may deflect the first latch arm 412 and the second latch arm 414 inwards and further movement will couple the first latch 416 and the second latch 418 with the corresponding latch features of the infusion portion 302, 320. Additionally or alternatively, the user may press the first lever 424 and the second lever 426 towards each other to deflect the first latch arm 412 and the second latch arm 414 and facilitate engagement of the first latch 416 and the second latch 418 with the corresponding latch features. Meanwhile, the needle 410 has connected with the fluid connector 308, for example by piercing a septum of the fluid connector 308 and forming a fluid connection with the cannula 310.

The tube connector 402 can be disconnected from the infusion portion 302, 320 by pressing the first lever 424 and the second lever 426 towards each other to disengage the first latch 416 and the second latch 418 from the corresponding latch features, and pulling the tube connector 402 away from the infusion portion 302, 320.

During connection and disconnection the first lever 424 and the second lever 426 advantageously provide improved contact area for a user to grip, and improved leverage for deflecting the first latch arm 412 and the second latch arm 414. In particular, the first lever 424 and the second lever 426 are arranged on the outside of the first latch arm 412 and the second latch arm 414, respectively, and the free portions 432, 434 elongate the first lever 424 and the second lever 426 further from the first flexing point 420 and the second flexing point 422. This advantageously reduces the force required to inwardly deflect the first latch arm 412 and the second latch arm 414. In addition, the elongated first lever 424 and second lever 426 may be gripped in different manners by a user, for example using the insides of their knuckles rather than their finger tips. Moreover, once connected the elongated first lever 424 and second lever 426 extend alongside the body portion 304, as shown in FIG. 2, and make it easier for a user to sense and regulate how much force to apply to the first lever 424 and the second lever 426 before pulling the tube connector 402 away. This is because the body portion 304 would limit the deflection of the first lever 424 and the second lever 426 and so the user would see that no excess force is needed. All of these advantages make it easier for a user with impaired movement or dexterity to use the tube connector 402 and the infusion set.

As described above, the tube connector 118, 212, 402 is used to connect a connecting tube 116, 214, 406 to an infusion portion 104, 204, 302, 320. The tube connector 118, 212, 402 has a connecting tube 116, 214, 406 attached to it and clips into the infusion portion 104, 204, 302, 320.

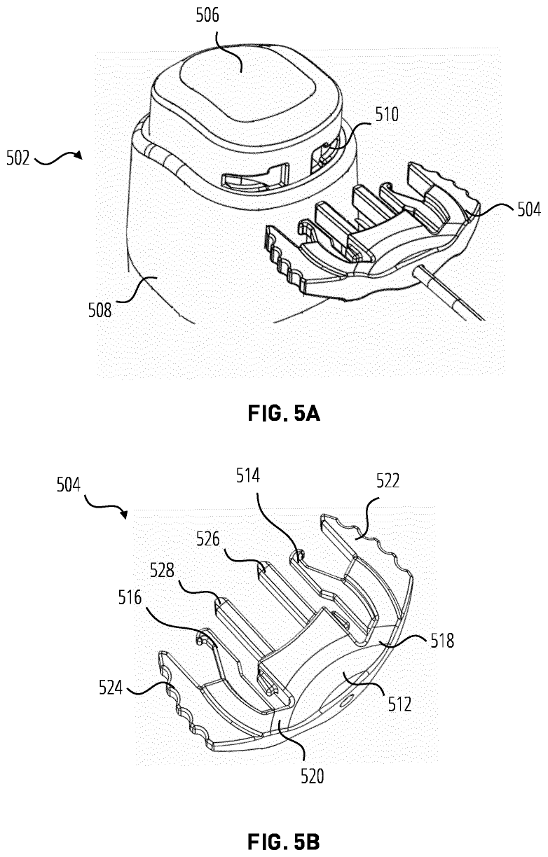

In other examples, as shown in FIG. 5A and FIG. 5B, a similar safety connector 504 can be used with an insertion device 502. The insertion device 502 shown in FIG. 5A is for attaching an infusion portion (e.g., the infusion portion 104, 204, 302, 320) to a user's skin 102 in the manner shown in FIG. 1A, so that the cannula 112 is inserted into a subcutaneous position. The insertion device 502 includes an insertion device body 508 that holds the infusion portion 104, 204, 302, 320 and the mounting portion 108 (see FIG. 1A). The infusion portion 104, 204, 302, 320 is held with the cannula directed towards an open end of the insertion device body 508. A button 506 is provided at the opposite end and pressing the button 506 pushes the infusion portion 104, 204, 302, 320 down through the insertion device body 508, out of the open end, and into contact with the user's skin. In this way the cannula pierces the skin and the mounting portion attaches the infusion portion 104, 204, 302, 320 to the skin.

Insertion devices 502 are known, for example, from EP2552513B1.

As shown in FIG. 5A, a safety connector 504 is used in connection portion 510 to lock the button 506 in an extended position before use of the insertion device 502. When the infusion system is provided to a user the infusion portion 104, 204, 302, 320 is provided within the insertion device body 508 of the insertion device 502, and the safety connector 504 is positioned to lock the button 506. Before use, the user removes the safety connector 504 from the connection portion 510, positions the insertion device 502 on their skin, and pushes the button 506 to insert the cannula and attach the infusion portion 104, 204, 302, 320 to the skin.

As shown in FIG. 5B, the safety connector 504 is similar to the tube connector 402 described above with reference to FIG. 4. The difference between the safety connector 504 and the 402 described with reference to FIG. 4 is that the safety connector 504 does not include a tube connection point or any needle or fluid path. However, in order to simplify manufacturing and reduce the number of unique components, the safety connector 504 may include a tube connection and fluid path which are not used.

The remainder of the safety connector 504 is as described with reference to FIG. 4 above. In particular, the safety connector 504 includes a connector body 512 from which extend a first latch arm 514 and a second latch arm 516. The first latch arm 514 and the second latch arm 516 are attached to the connector body 512 at a first flexing point 518 and a second flexing point 520, respectively. A first lever 522 is joined to the first latch arm 514 and a second lever 524 is joined to the second latch arm 516. The first lever 522 and the second lever 524 are as described with reference to the tube connector 402 of FIG. 4. The safety connector 504 also includes a first guide arm 526 and a second guide arm 528.

As shown in FIG. 5A, the insertion device 502 includes a connection portion 510 that receives the safety connector 504. In particular, the connection portion 510 includes an opening that receives the first latch arm 514, the second latch arm 516, the first guide arm 526 and the second guide arm 528. When the safety connector 504 is connected to the connection portion 510 the first lever 522 and the second lever 524 would be external and partially surrounding the button 506. In this way, when the safety connector 504 is connected to the connection portion 510 it prevents downward movement of the button 506, thereby preventing accidental or early actuation of the insertion device 502.

As mentioned above, before using the insertion device 502 the safety connector 504 is removed, allowing the button 506 to be depressed. Therefore, the advantages of the tube connector 402 described above, in terms of case of handling for removal, apply to the safety connector 504 also. In addition, it is advantageous to provide the same connector as the safety connector 504 and as the tube connector 402 so that the patient does not have to use two different types of connector.

As mentioned above, the tube connector 118, 212, 412 can be coupled via the connecting tube 214 to a pump for administrating a therapeutic substance, for example. However, the tube connector 118, 212, 412 can be replaced with a dummy connector or dummy part, which dummy part is shaped to correspond to the tube connector 118, 212, 412 but without the connecting tube 214. In this way, a patient can disconnect the real tube connector 118, 212, 412 and replace the real tube connector 118, 212, 412 with the dummy part. In this way, the pump for administrating medicines or a therapeutic substances can be disconnected from the patient's infusion set 202. When the patient is wearing the infusion set 202 with the dummy part or dummy connector and is in need of medication or a therapeutically substance, the patient can e.g. inject the medication or a therapeutically substance through a part of the 202 directly into the cannula 210, for example by using a syringe.

In this way, it is easier for the patient to leave the house for a shorter period of time without the inconvenience of having to bring unmanageable medical equipment such as the above mentioned pump for administrating medicines, but still be able to inject medicines or a therapeutic substance by use of e.g., a small syringe when needed.

Similarly, the safety connector 504 illustrated in FIG. 5A and FIG. 5B may be used as the dummy part after being removed from the insertion device 502.

In some examples, the safety connector 504 may additionally include the fluid path 408 and the needle 410 shown in FIG. 4, and a pierceable region, for example a septum, located at the opening of the fluid path 408. In this way, the septum may allow a patient to administer medicament by injecting medicament through the fluid path 408 and needle 410 and into the infusion set 202.

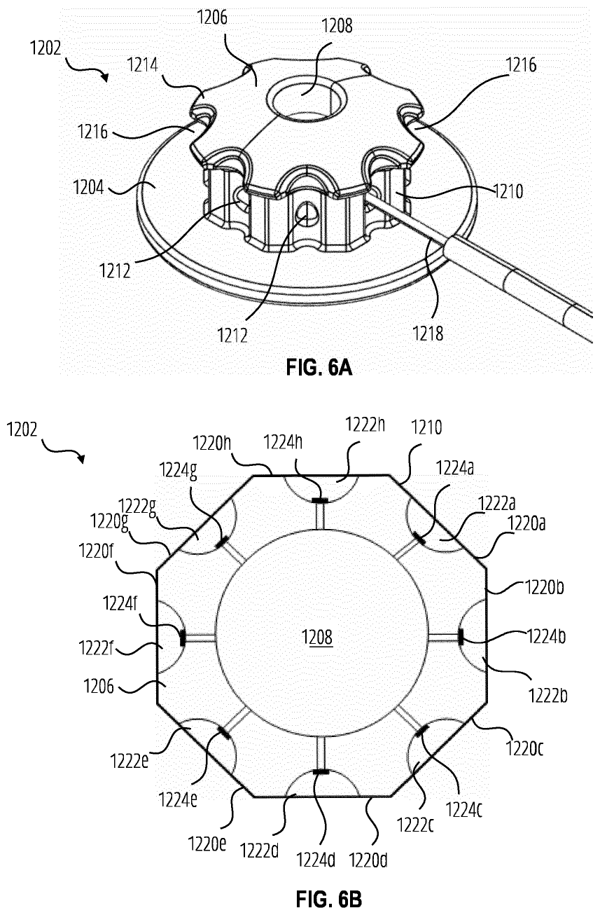

FIG. 6A shows an infusion portion 1202 of an infusion set. The infusion portion 1202 includes a base portion 1204 and a spigot 1206 protruding from the base portion 1204. In use, the base portion 1204 is attached to a patient's skin, for example using an adhesive patch as described with reference to FIG. 1A. The infusion portion 1202 includes a cannula in cannula opening 1208. A side wall 1210 of the spigot 1206 includes a plurality of fluid ports 1212. Each fluid port 1212 is in fluid communication with the cannula through the cannula opening 1208, and may include a seal, for example a septum. Accordingly, a needle 1218 of a tube connector may be connected to any of the fluid ports 1212 for fluidly connecting with the cannula.

The spigot 1206 also includes a flange edge 1214. The flange edge 1214 has a plurality of recesses 1216 formed about its peripheral edge. In this way, the flange edge 1214 has an undulating form. Each fluid port 1212 is in alignment with a recess 1216 of the flange edge 1214. As illustrated, the recesses 1216 may extend through the side wall 1210 as well, as described further below.

Accordingly, a tube connector can be attached to the infusion portion 1202 from any of a number of different directions in which the needle 1218 aligns with a fluid port 1212 in the infusion portion 1202. As described below, the shape of the side wall 1210 and/or the flange edge 1214 ensures correct alignment between the tube connector and one of the fluid ports 1212.

FIG. 6B shows a cross-section through the infusion portion 1202, in particular through the spigot 1206 below the flange edge 1214. As shown, the side wall 1210 of the spigot 1206 has a polygonal form. That is, the cross-section of the side wall 1210 is polygonal. In particular, the cross-section of the side wall 1210 has a regular polygonal cross-section with an even number of sides. Specifically, the side wall 1210 has a number of sides 1220a-1220h that is exactly divisible by four. The cross-section of the side wall 1210 is therefore regular and symmetrical, and the sides are arranged in parallel opposing pairs of sides with identical lengths.

In the illustrated example the side wall 1210 has eight sides 1220a-1220h. Each side 1220a-1220h has a recess 1222a-1222h, and a fluid port 1224a-1224h. In other examples, the side wall 1210 may have four sides, or twelve sides, or sixteen sides, each with a fluid port. Each side 1220a-1220h may or may not have a recess as shown in FIG. 6B.

Each fluid port 1224a-1224h is connected to the cannula opening 1208, which is connected to the cannula. In this way, any of the fluid ports 1224a-1224h can be used to connect to the cannula. Each fluid port 1224a-1224h comprises a seal, for example a septum, that can be punctured by a needle as described below.

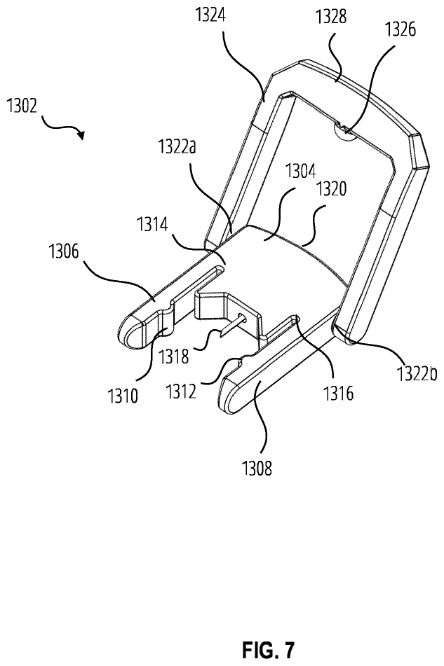

FIG. 7 illustrates a tube connector 1302 for use with the infusion portion 1202 of FIG. 6A and FIG. 6B.

As shown in FIG. 7, the tube connector 1302 includes a connector body 1304. The connector body 1304 includes a tube connection point 1320 where a tube (not shown) is connected. The connector body 1304 also includes a needle 1318 and a fluid path that extends between the needle 1318 and the tube connection point 1320. When the tube connector 1302 is connected to an infusion portion the fluid path provides a fluid connection between the tube and the cannula as described previously. In other examples, instead of the needle 1318 the tube connector 1302 may include a tube connection point for fluidly connecting with a fluid port or needle of the infusion portion.

As shown, the tube connector 1302 includes a locking member, in this example a loop member 1324. The loop member 1324 is rotatably mounted to the connector body 1304 at hinges 1322a, 1322b. The loop member 1324 is rotatable relative to the connector body 1304 so that the loop member 1324 can be used as a pull ring and/or as a locking mechanism, as described below. The loop member 1324 includes a clip portion 1326 for clipping the loop member 1324 in a locked position, as described further below. The loop member 1324 also includes a finger grip 1328 for easier gripping of the loop member 1324 for lifting it from a locked position, as described further below.

As shown in FIG. 7, the tube connector 1302 includes a first arm 1306 and a second arm 1308. The first arm 1306 and the second arm 1308 extend from the connector body 1304 The first arm 1306 and the second arm 1308 are substantially parallel to each other. The needle 1318 is disposed between the first arm 1306 and the second arm 1308 and directed in substantially the same direction as the first arm 1306 and the second arm 1308. In examples where the needle 1318 includes a needle, the needle extends parallel to the first arm 1306 and the second arm 1308 and between the first arm 1306 and the second arm 1308. As described below, this allows the tube connector 1302 to the connected to the infusion portion by a sideways sliding movement that forms a fluid connection with the needle 1318 while the first arm 1306 and the second arm 1308 couple the tube connector 1302 to the infusion portion.

In the illustrated example the first arm 1306 includes a first latch 1310 and the second arm 1308 includes a second latch 1312. The first latch 1310 is directed inwardly, towards from the second arm 1308 and the second latch 1312 is directed inwardly, towards the first arm 1306.

The first arm 1306 is joined to the connector body 1304 at a first flexing point 1314. The second arm 1308 is joined to the connector body 304 at a second flexing point 1316. The first flexing point 1314 and the second flexing point 1316 are resiliently deformable such that the first arm 1306 and the second arm 1308 are resiliently deflectable, allowing the first latch 1310 and the second latch 1312 to be latched and unlatched. The first flexing point 1314 and the second flexing point 1316 may be formed by a thinner section of the connector body 1304, or by a different material (e.g., provided by co-moulding), or by an embedded or attached feature such as a spring feature.

In some alternative examples the first arm 1306 and the second arm 1308 may not include any latches. In such examples, the loop member 1324 may be relied on to hold the tube connector 1302 in connection with the infusion portion 1202.

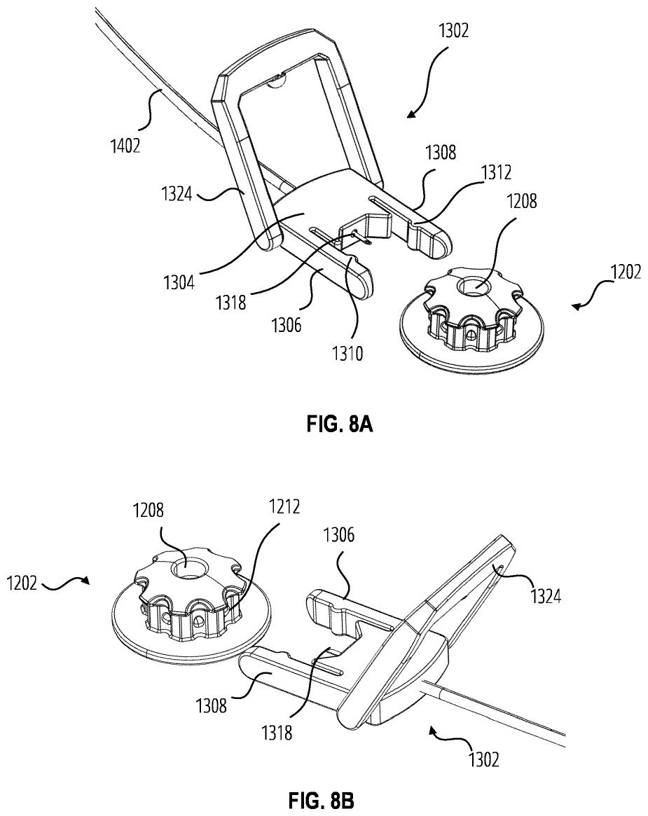

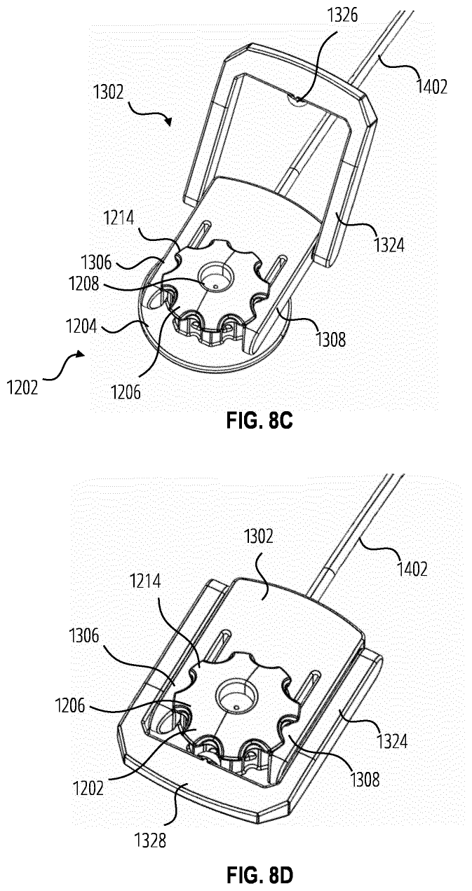

FIG. 8A to FIG. 8D illustrate a tube connector 1302 described with reference to FIG. 7 being connected to the infusion portion 1202 described with reference to FIG. 6A and FIG. 6B. In FIG. 8A to FIG. 8D the tube connector 1302 includes a needle 1318 and a connecting tube 1402. The needle 1318 extends from the connector body 1304 between the first arm 1306 and the second arm 1308. The connecting tube 1402 is connected on the opposite side of the connector body 1304 and is fluidly connected to the needle 1318 through a fluid path in the connector body 1304. When the tube connector 1302 is connected to the infusion portion 1202 the needle 1318 provides a fluid connection between the connecting tube 1402 and the cannula in the cannula opening 1208 as described previously.

As shown in FIG. 8A, before the tube connector 302 is connected to the infusion portion 1202 the loop member 1324 is rotated away from the infusion portion 1202, into an open position that allows the tube connector 1302 to be slid sideways onto the infusion portion 1202 as described below.

As shown in FIG. 8B, during connection of the tube connector 1302 and the infusion portion 1202 the needle 1318 is aligned with a fluid port 1212 of the infusion portion 1202. The needle 1318 passes into the fluid port 1212 to form a fluid connection between the connecting tube (1402, see FIG. 8A) and the cannula in the cannula opening 1208.

Referring to FIG. 8B and FIG. 8C, as the tube connector 1302 is pushed onto the infusion portion 1202, with the needle 1318 in a fluid port 1212, the first arm 1306 and the second arm 1308 are positioned either side of the spigot 1206 of the infusion portion 1202. The first arm 1306 and the second arm 1308 are positioned between the flange edge 1214 and the base portion (1204, see FIG. 6A).

In particular, the first arm 1306 and the second arm 1308 contact opposite sides of the spigot 1206. The first arm 1306 and the second arm 1308 are spaced to only allow the tube connector 1302 to be slid onto the infusion portion 1202 if the first arm 1306 and the second arm 1308 are parallel to two opposite sides of the spigot 1206 (e.g., side 1220a and side 1220c as shown in FIG. 6B). Due to the regular polygonal cross-section of the spigot 1206 having a number of sides 1220a-1220h that is exactly divisible by four (see description of FIG. 6B), and the location of the fluid ports 1224a-1224h in each side 1220a-1220h, the tube connector 1302 can only be connected to the infusion portion 1202 when the needle 1318 is aligned with a fluid port 1224a-1224h.

Accordingly, the shape of the infusion portion 1202, in particular the spigot 1206, allows the tube connector 1302 to be connected by a sideways sliding movement from a number of different directions. In the illustrated example, the tube connector 1302 can be connected to the infusion portion 1202 from eight different directions. The shape of the infusion portion 1202 also prevents the tube connector 1302 from being pushed onto the infusion portion 1202 unless there is correct alignment.

Additionally, the tube connector 1302 may include a protrusion (not illustrated) that aligns with, and engages, a recess (1216, see FIG. 6A) of the infusion portion 1202, particularly on the flange edge (1214, see FIG. 6A). This can provide further alignment between the needle 1318 and the fluid port (1212, see FIG. 8B) of the infusion portion 202.

As shown in FIG. 8C, when the tube connector 1302 is pushed onto the infusion portion 1202 the first arm 1306 and the second arm 1308 are positioned either side of the spigot 1206. In examples where the first arm 1306 includes a first latch 1310 and the second arm 1308 includes a second latch 1312, the first latch 1310 and the second latch 1312 may clip over the side wall (1210, see FIG. 6A) of the spigot 1206 to hold the tube connector 1302 on the infusion portion 1202. In particular, the first latch 1310 and the second latch 1312 may engage opposing recesses (recesses 1222a-1222h, see FIG. 6B) of the infusion portion 1202. However, in some examples the first latch 1310 and the second latch 1312 are omitted.

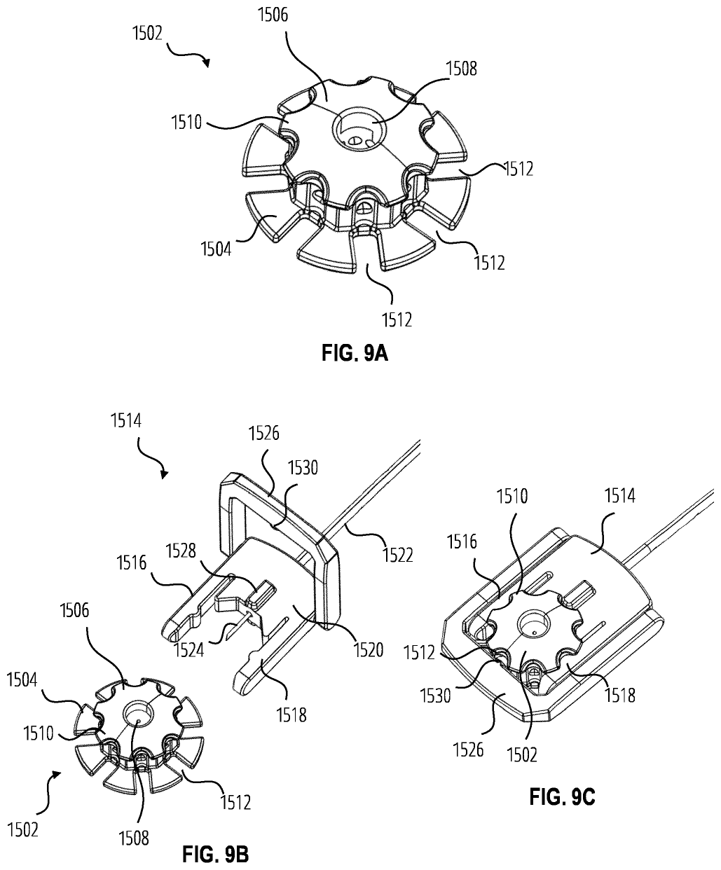

As shown in FIG. 8D, once the needle 1318 is connected with the fluid port (1212, see FIG. 9B), the loop member 1324 can be rotated down to surround the spigot 1206 of the infusion portion 1202. In this position the tube connector 1302 is locked onto the infusion portion 1202 by the loop member 1324.

As described above, the loop member 1324 may have some interference with the spigot 1206, in particular the flange edge 1214, as it is rotated over the spigot 1206. The loop member 1324 may include one or more clip features (1326, see FIG. 8C) that clip over the flange edge 1214 as the loop member 1324 is pushed down over the spigot 1206. This may improve locking by holding the loop member 1324 in the locked position shown in FIG. 7D.

A patient can unlock the tube connector 1302 from the infusion portion 1202 by rotating the loop member 1324 away from the infusion portion 1202. The finger grip 1328 may make it easier for the patient to grip the loop member 1324.

Advantageously, only a small force is required to connect or disconnect the tube connector 1302 from the infusion portion 1202, making it easier for user with impaired movement or dexterity.

FIG. 9A shows a further example infusion portion 1502 of an infusion set. The infusion portion 1502 is the similar to the infusion portion 1202 described with reference to FIG. 6A and FIG. 6B. In particular, the infusion portion 1502 includes a base portion 1504 and a spigot 1506. The spigot 1506 includes a cannula opening 1508 and there are a plurality of fluid ports (not shown in FIG. 9A) in a side wall of the spigot 1506. A flange edge 1510 of the spigot 1506 is provided. The spigot 1506, in particular the side wall, has a regular polygonal cross-section with a number of sides that is exactly divisible by four. The side wall includes a plurality of fluid ports, as described with reference to FIG. 6A and FIG. 6B.

As also shown in FIG. 9A, in this example the base portion 1504 includes a plurality of recesses 1512 spaced about the base portion 1504. The recesses 1512 of the base portion 1504 are aligned with the recesses (1216, see FIG. 6A) of the flange edge 1510, and the recesses (1222a-1222h, see FIG. 6B) of the spigot 1506. The recesses 1512 are thereby also aligned with the fluid ports in the spigot 1506.

As shown in FIG. 9B and FIG. 9C, a tube connector 1514 can be connected to the infusion portion 1502. The tube connector 1514 is similar to the tube connector 1302 described with reference to FIG. 7 above. The tube connector 1514 can be connected to the infusion portion 1502 in the same manner as described in detail with reference to FIG. 8A to FIG. 8D.

In particular, as illustrated, the tube connector 1514 comprises a connector body 1520 with a first arm 1516 and a second arm 1518 extending therefrom. The first arm 1516 and the second arm 1518 are positioned either side of the spigot 1506 of the infusion portion 1502, below the flange edge 1510, when the tube connector 1514 is connected to the infusion portion 1502. As also shown, the tube connector 1514 includes a connecting tube 1522 that is in communication with a needle 1524 that forms a fluid connection with a cannula in the cannula opening 1508 when the tube connector 1514 is connected to the infusion portion 1502. A rotatable locking member, in particular a loop member 1526, is provided for pulling and for locking the tube connector 1514 to the infusion portion 1502 as described above.

As illustrated in FIG. 9B and FIG. 9C, the tube connector 1514 also includes a guide 1528. In this example the guide 1528 is a protrusion. The guide 1528 is disposed on the connector body 1520 and is proximate to the needle 1524. As shown in FIG. 9B and FIG. 9C, when the tube connector 1514 is connected to the infusion portion 1502 the guide 1528 is aligned with one of the recesses 1512 of the flange edge 1510. In this way, the guide 1528 cooperates with a recess 1512 to ensure proper alignment when the tube connector 1514 is attached to the infusion portion 1502.

In a similar manner, the guide 1528 may extend from the opposite side of the connector body 1520 to that illustrated, so that the guide 1528 may additionally or alternative engage a recess 1512 of the base portion 1504 to ensure alignment in the same way.

Accordingly, the tube connector 1514 can be attached to the infusion portion 1502 from a number of different directions in which the needle 1524 aligns with a fluid port (1212, see FIG. 6A) in the infusion portion 1502.

As also shown in FIG. 9B and FIG. 9C, the loop member 1526 may include a clip 1530 that interacts with the infusion portion 1502 to clip the loop member 1526 into the closed position shown in FIG. 9C, as previously described. In this example the clip 1530 is located centrally in the loop member 1526 and arranged to clip into a recess 1512 of the spigot 1506 when the loop member 1526 is in the closed position shown in FIG. 9C.

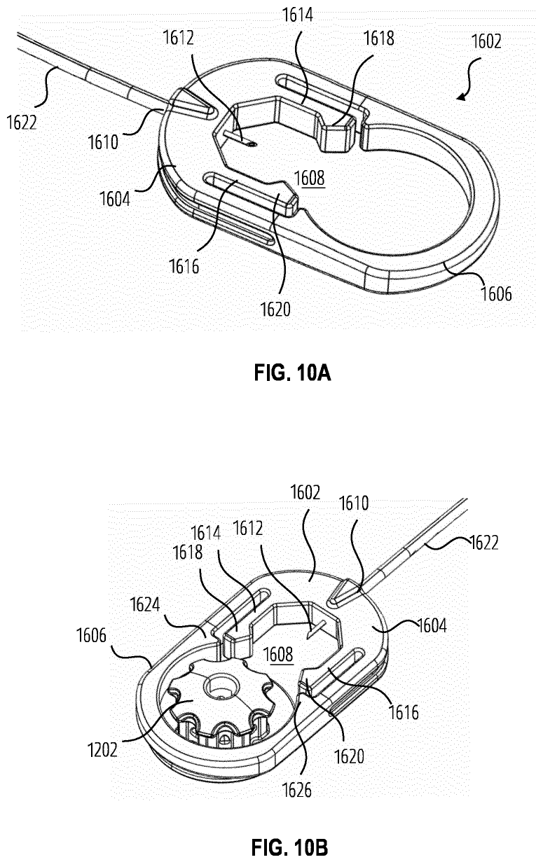

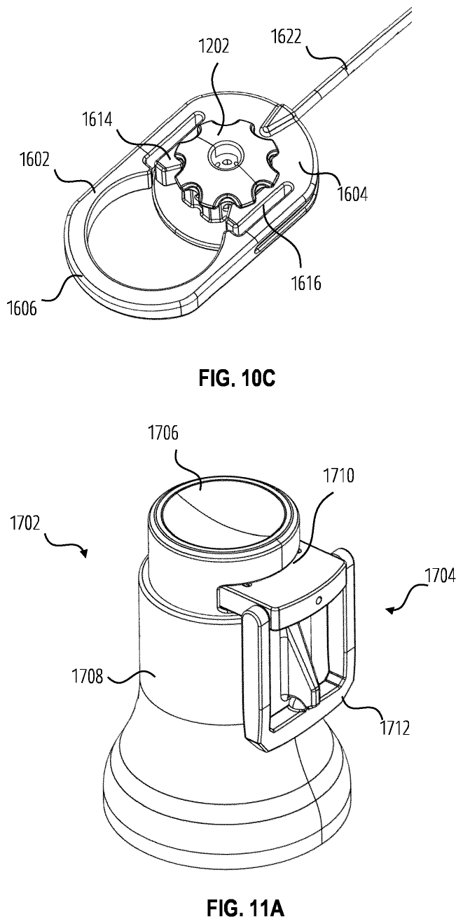

FIG. 10A illustrates a further example tube connector 1602 for use with the infusion portion 1202 illustrated in FIG. 6A and FIG. 6B and/or the infusion portion 1502 illustrated in FIG. 9A. In this example, the tube connector 1602 includes a first loop member 1604 and a second loop member 1606 joined to each other.

The first loop member 1604 and the second loop member 1606 are partial circles, with an opening 1608 formed between them so that the first loop member 1604 and the second loop member 1606 define a closed shape. The first loop member 1604 is shaped to match the infusion portion 1202, in particular the spigot (1206, see FIG. 6A and FIG. 6B). In particular, the first loop member 1604 includes a first arm 1614 and the second arm 1616 arranged on opposite sides that engage the infusion portion 1202. The first arm 1614 and the second arm 1616 are connected to the first loop member 1604 at flexing points so that the first arm 1614 and the second arm 1616 are resiliently deflectable. The first arm 1614 includes a first latch 1618 facing the second arm 1616, and the second arm 1616 includes a second latch 1620 facing the first arm 1614.

The second loop member 1606 is larger than the first arm 1614.

The tube connector 1602 also includes a tube connection point 1610 for attaching a connecting tube 1622 as shown in FIG. 10B. The tube connector 1602 also includes a needle 1612 that forms a fluid connection with the infusion portion 1202 when attached. The needle 1612 may include a needle 1612 as shown in FIG. 10B.

FIG. 10B shows the tube connector 1602 of FIG. 10A in an initial stage of being connected to the infusion portion 1202 described with reference to FIG. 6A and FIG. 6B. FIG. 10C shows the tube connector 1602 connected to the infusion portion 1202. It will be appreciated that the tube connector 1602 can be connected to the infusion portion 1502 of FIG. 9A in the same manner.

As shown in FIG. 10B, initially the second loop member 1606 is placed over the infusion portion 1202. The second loop member 1606 is large enough to be placed over the infusion portion 1202 without engaging the infusion portion 1202. As shown, in this position the needle 1612 is directed towards the infusion portion 1202. The first arm 1614 and the second arm 1616 are also directed towards the infusion portion 1202.

From this position the tube connector 1602 can be slid sideways, towards the infusion portion 1202, to connect the tube connector 1602 to the infusion portion 1202. As shown in FIG. 10B, the opening 1608 is narrowed at the intersect between the first loop member 1604 and the second loop member 1606 by protrusion 1624 and opposing protrusion 1626. This narrowing of the opening 1608 helps to guide the infusion portion 1202 into the first loop member 1604.

FIG. 10C shows the tube connector 1602 when connected to the infusion portion 1202. The tube connector 1602 has been slid sideways from the position shown in FIG. 10B. The first arm 1614 and the second arm 1616 have engaged the infusion portion 1202, in particular the side wall (1210, see FIG. 6A and FIG. 6B) of the spigot (1206, see FIG. 6A and FIG. 6B). The needle 1612 has also engaged a fluid port (1212, see FIG. 6A and FIG. 6B) to form a fluid connection between the connecting tube 1622 and the infusion portion 1202.

The first arm 1614 and the second arm 1616 are spaced such that the movement from the position shown in FIG. 10B to the position shown in FIG. 10C is only possible if the needle 1612 is in alignment with a fluid port of the infusion portion 1202. That is, the first arm 1614 and the second arm 1616 engage opposite sides of the side wall (1210, see FIG. 6B) and prevent movement of the tube connector 1602 towards the infusion portion 1202 unless the first arm 1614 and the second arm 1616 are parallel to opposite sides of the side wall (1210, see FIG. 6B). In this way, the tube connector 1602 can only be slid into connection with the infusion portion 1202 if the needle 1612 is aligned with a fluid port of the infusion portion 1202.

As the tube connector 1602 is slid into connection with the infusion portion 1202 the first latch 1618 and the second latch 1620 engage the infusion portion 1202 and deflect the first arm 1614 and the second arm 1616 outwards. The first latch 1618 and the second latch 1620 can then clip around the infusion portion 1202 to hold the tube connector 1602 in position on the infusion portion 1202.

Advantageously, the second loop member 1606 can be used by a patient to grip the tube connector 1602 for disconnecting the tube connector 1602 from the infusion portion 1202. In particular, a patient may grip any part of the second loop member 1606 and push it towards the infusion portion 1202 to disconnect the tube connector 1602 from the infusion portion 1202. Similarly, the second loop member 1606 may act as a pull ring to move the tube connector 1602 into connection with the infusion portion 1202. A patient with impaired dexterity may find it easier to grip and push/pull the second loop member 1606, making it easier for them to connect and disconnect the tube connector 1602.

As described above, the tube connector 118, 1302, 1514 is used to connect a connecting tube 116, 1402, 1522 to an infusion portion 104, 1202, 1502. The tube connector 118, 1302, 1514 has a connecting tube 116, 1402, 1522 attached to it and clips into the infusion portion 104, 1202, 1502.

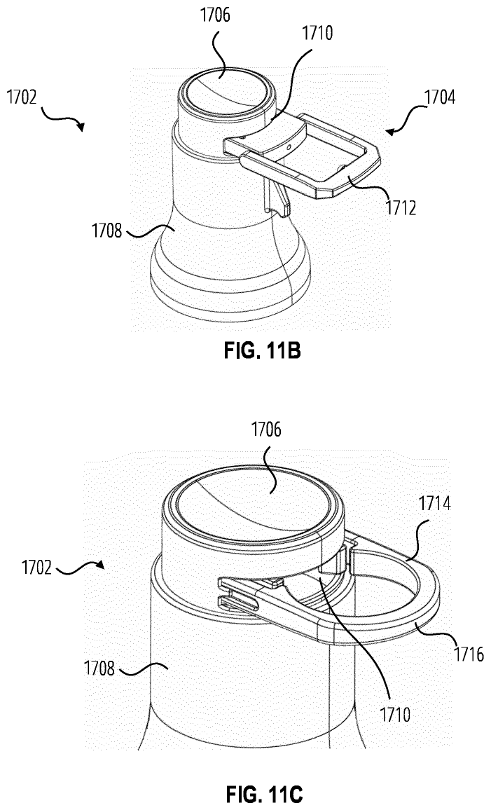

In other examples, as shown in FIG. 11A, a connector similar to the tube connector 118, 1302, 1514 described above may be provided as a safety connector 1704. The safety connector 1704 can be used with an insertion device 1702. The insertion device 1702 shown in FIG. 11A is for attaching an infusion portion (e.g., the infusion portion 104, 1202, 1502) to a user's skin 102 in the manner shown in FIG. 1A, so that the cannula 112 is inserted into a subcutaneous position. The insertion device 1702 includes an insertion device body 1708 that holds the infusion portion 104, 1202, 1502 and the mounting portion 108 (see FIG. 1A). The infusion portion 104, 1202, 1502 is held with the cannula directed towards an open end of the insertion device body 1708. A button 1706 is provided at the opposite end and pressing the button 1706 pushes the infusion portion 104, 1202, 1502 down through the insertion device body 1708, out of the open end, and into contact with the user's skin. In this way the cannula pierces the skin and the mounting portion attaches the infusion portion 104, 1202, 1502 to the skin.

Suitable insertion devices 1702 are known, for example, from EP2552513B1.

As shown in FIG. 11A, a safety connector 1704 is used in connection portion 1710 to lock the button 1706 in an extended position before use of the insertion device 1702. When the infusion system is provided to a user the infusion portion 104, 1202, 1502 is provided within the insertion device body 1708 of the insertion device 1702, and the safety connector 1704 is positioned to lock the button 1706. The safety connector 1704 is substantially the same as the tube connector 1302 described with reference to FIG. 7, or it may be substantially the same as the tube connector 1514 described with reference to FIG. 9B and FIG. 9C. However, the safety connector 1704 does not have a tube or a needle. In this position the loop member 1712 of the safety connector 1704 is folded against the insertion device body 1708.

Before use, the user removes the safety connector 1704 from the connection portion 1710 by rotating the loop member 1712 upwards and using the loop member 1712 as a pull ring to pull the safety connector 1704 from the connection portion 1710. The user can then position the insertion device 1702 on their skin, and push the button 1706 to insert the cannula and attach the infusion portion 104, 1202, 1502 to the skin.

In particular, as shown in FIG. 11B the loop member 1712 can be rotated away from the insertion device body 1808 so that it can be gripped for pulling the safety connector 1704 out of the connection portion 1710 and away from the insertion device 1702. After removal of the safety connector 1704 the button 1706 can be depressed to operate the insertion device 1702.

Therefore, the advantages of the tube connector 118, 1302, 1514 described above, in terms of case of handling for removal, apply to the safety connector 1704 also. In addition, it is advantageous to provide the same connector as the safety connector 1704 and as the tube connector 118, 1302, 1514 so that the patient does not have to use two different types of connector. In particular, a patient with impaired dexterity may find it easier to grip the loop member 1712, either by using it as a pull ring or by gripping opposite sides of the loop member 1712.

FIG. 11C shows insertion device 1702 with an alternative safety connector 1714. In particular, the safety connector 1714 is the same as the tube connector 1602 described with reference to FIG. 10A, but without a needle 1612 or connecting tube 1622. The safety connector 1714 includes a second loop member 1716 that is the same as the second loop member 1606 described with reference to FIG. 10A. As shown in FIG. 11C, the second loop member 1716 provides a pull ring for removing the safety connector 1714 from the insertion device 1702, allowing the button 1706 to be depressed for deploying the infusion portion as described above.

As mentioned above, the tube connector 118, 1302, 1514, 1602 can be coupled via a connecting tube 116, 1402, 1522, 1622 to a pump for administrating a therapeutic substance, for example. However, the tube connector 118, 1302, 1514, 1602 can be replaced with a dummy connector or dummy part, which dummy part is shaped to correspond to the tube connector 118, 1302, 1514, 1602 but without the connecting tube 116, 1402, 1522, 1622. In this way, a patient can disconnect the real tube connector 118, 1302, 1514, 1602 and replace the real tube connector 118, 1302, 1514, 1602 with the dummy part. In this way, the pump for administrating medicines or a therapeutic substances can be disconnected from the patient's infusion portion 104, 1202, 1502. When the patient is wearing the infusion portion 104, 1202, 1502 with the dummy part or dummy connector and is in need of medication or a therapeutically substance, the patient can e.g. inject the medication or a therapeutically substance through a part of the infusion portion 104, 1202, 1502 directly into the cannula, for example by using a syringe.

In this way, it is easier for the patient to leave the house for a shorter period of time without the inconvenience of having to bring unmanageable medical equipment such as the above mentioned pump for administrating medicines, but still be able to inject medicines or a therapeutic substance by use of e.g., a small syringe when needed.

Similarly, the safety connector 1704, 1714 illustrated in FIG. 11A, FIG. 11B or FIG. 11C may be used as the dummy part after being removed from the insertion device 1702.

In some examples, the safety connector 1704, 1714 may fluidly couple to the infusion portion 104, 1202, 1502 and may include a pierceable region, for example a septum, provided to allow a patient to administer medicament by injecting medicament through the pierceable region and into the infusion portion 104, 1202, 1502.

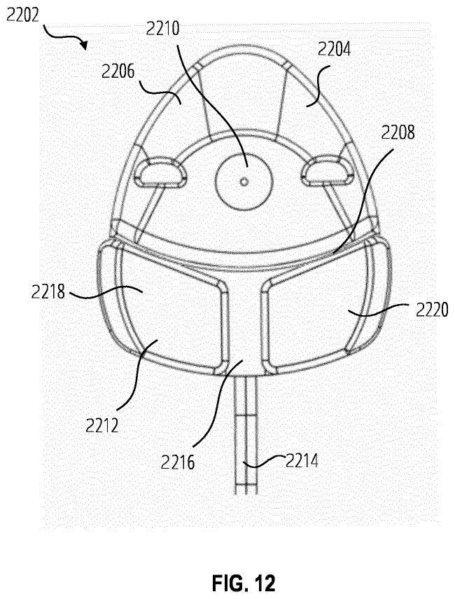

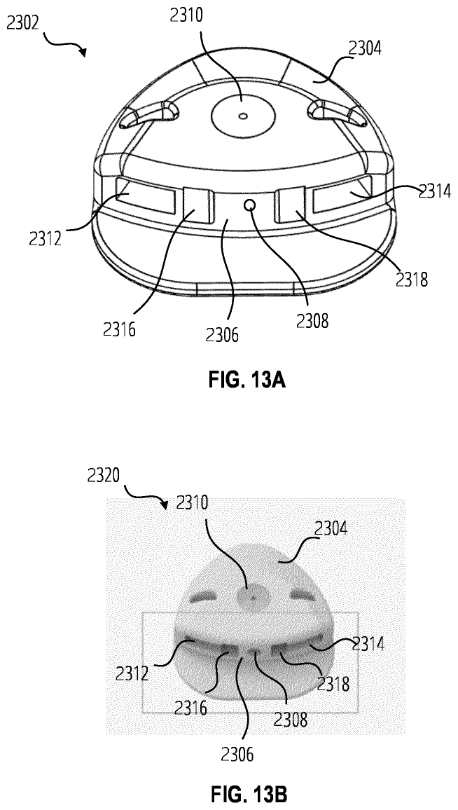

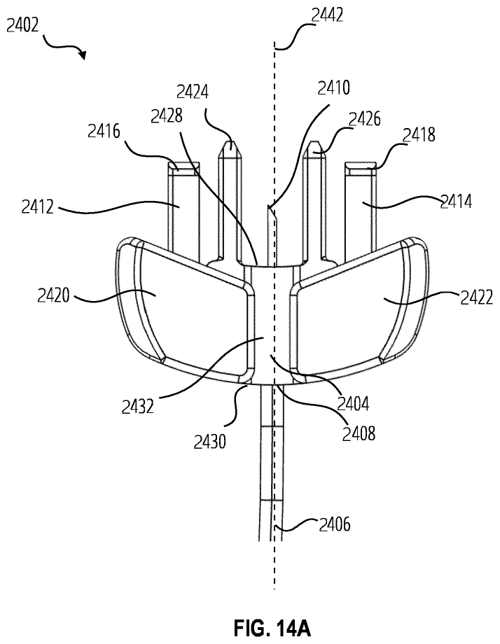

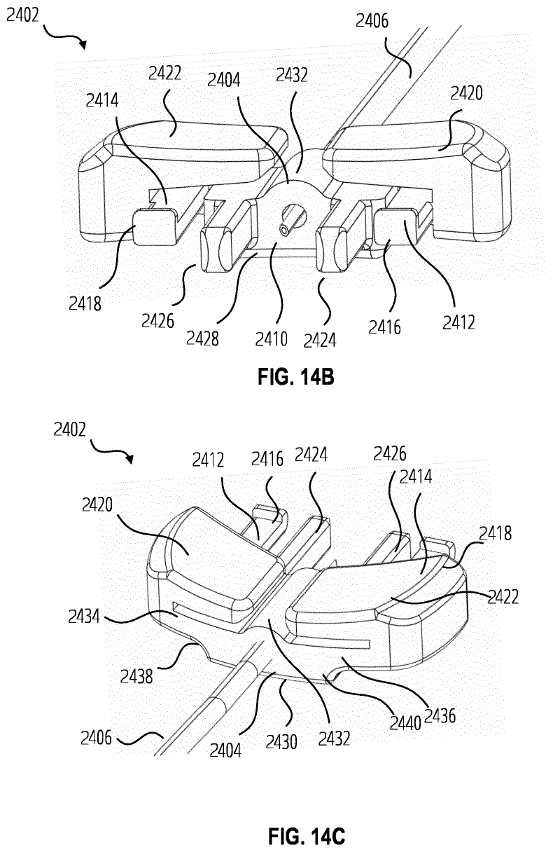

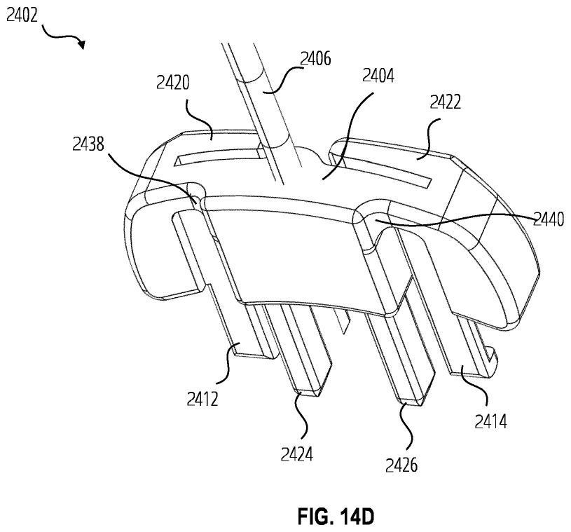

FIG. 12 illustrates an example infusion set 2202. The infusion set 2202 includes an infusion portion 2204. The infusion portion 2204 has a body portion 2206 that is attachable to a user as described with reference to FIG. 1A to FIG. 1C. A cannula 2210 is provided within the body portion 2206 and the cannula 2210 is subcutaneously positioned when the body portion 2206 is attached to the user. It will be appreciated that the cannula 2210 protrudes from the opposite side of the body portion 2206 to that illustrated, and the top of cannula 2210 is indicated in FIG. 12. The infusion portion 2204 also includes a tube connecting portion 2208 for connecting with a connecting tube 2214 via a tube connector 2212. The tube connecting portion 2208 and tube connector 2212 are described in more detail with reference to FIG. 13A to FIG. 14A.

As shown, the tube connector 2212 includes a body portion 2216, a first tab 2218 and a second tab 220. The first tab 2218 and the second tab 2220 overlie the body portion 2216 and, as described further below, the first tab 2218 and the second tab 2220 can be pushed towards the body portion 2216 to move latching arms and disconnect tube connector 2212 from the infusion portion 2204.

FIG. 13A illustrates a first example infusion portion 2302 that may be the infusion portion 2204 described with reference to FIG. 12. As shown, the infusion portion 2302 includes a body portion 2304 that is attachable to a user as described with reference to FIG. 1A to FIG. 1C. A cannula 2310 is provided within the body portion 2304 and the cannula 2310 is subcutaneously positioned when the body portion 2304 is attached to the user. It will be appreciated that the cannula 2310 protrudes from the opposite side of the body portion 2304 to that illustrated, and the top of cannula 2310 is indicated in FIG. 12. The infusion portion 2302 also includes a tube connecting portion 2306 for connecting with a connecting tube via a tube connector as described further with reference to FIG. 14A.

The tube connecting portion 2306 includes a fluid connector 2308 for forming a fluid connection with the tube connector and connecting tube shown in FIG. 14A. The fluid connector 2308 may include a septum or other seal that is pierced by the tube connector, and allows for resealing the fluid connector 2308 when the tube connector is disconnected from the infusion portion 2302.

As shown in FIG. 13A, the tube connecting portion 2306 includes a first latch opening 2312 and a second latch opening 2314 that receive first and second latch arms of the tube connector described further with reference to FIG. 14A. The first latch opening 2312 and the second latch opening 2314 include internal latch portions as described further below. The tube connecting portion 2306 also includes a first guide opening 2316 and a second guide opening 2318. The first guide opening 2316 and the second guide opening 2318 are arranged to receive first and second guide arms of the tube connector described further with reference to FIG. 14A.

FIG. 13B illustrates a further example infusion portion 2320 that may be the infusion portion 2204 described with reference to FIG. 12. The infusion portion 2320 of FIG. 13B is similar to that of FIG. 13A and the same reference numerals are used for alike features. The difference between the infusion portion 2302 of FIG. 13A and the infusion portion 2320 of FIG. 13B is that the first latch opening 2312 and the first guide opening 2316 are formed as separate parts of a single opening in the body portion 2304. Similarly, the second latch opening 2314 and the second guide opening 2318 are formed as separate parts of a single opening in the body portion 2304. In particular, the first latch opening 2312 is formed in a first part of the opening arranged generally outwardly from the fluid connector 2308, and the first guide opening 2316 is arranged in a second part of the opening arranged adjacent to the fluid connector 2308. In the same manner, the second latch opening 2314 is formed in a first part of the opening arranged generally outwardly from the fluid connector 2308, and the second guide opening 2318 is arranged in a second part of the opening arranged adjacent to the fluid connector 2308.

FIG. 14A to FIG. 14D illustrate a tube connector 2402 for use with the infusion portion 2302 of FIG. 13A and the infusion portion 2320 of FIG. 13B.

As shown in FIG. 14A, the tube connector 2402 includes a body portion 2404. The body portion 2404 includes a front side 2428 and a rear side 2430. A fluid connector is provided on the front side 2428. In this example, the fluid connector comprises a needle 2410 extending from the front side 2428. The tube connector 2402 also includes a connecting tube 2406 extending from a tube connection point on the rear side 2430 of the body portion 2404 and is connected to the needle 2410. The needle 2410 and connecting tube 2406 are connected by a fluid path 2408 extending through the body portion 2404. The body portion 2404 is symmetrical about a mid-plane 2442 extending parallel with the connecting tube 2406 and the needle 2410.

When the tube connector 2402 is connected to the infusion portion 2302 (FIG. 13A) or infusion portion 2320 (FIG. 13B) the needle 2410 engages the fluid connector 2308 to form a fluid connection between the connecting tube 2406 and the cannula 2310.

As shown in FIG. 14A to FIG. 14D, the body portion 2404 is generally planar and extends either side of the needle 2410 and the connecting tube 2406. The body portion 2404 includes a central part 2432 where the needle 2410 and the connecting tube 2406 are connected. The tube connector 2402 includes a first latch arm 2412 and a second latch arm 2414. The first latch arm 2412 and the second latch arm 2414 are joined to opposite sides of the body portion 2404. The first latch arm 2412 and the second latch arm 2414 protrude substantially parallel to each other, and substantially parallel to the needle 2410. The first latch arm 2412 and the second latch arm 2414 are arranged in the same plane as the body portion 2404, which is perpendicular to the mid-plane 2442 of the tube connector 2402.

The first latch arm 2412 includes a first latch 2416 at a distal end of the first latch arm 2412 (the end opposite to the body portion 2404). The second latch arm 2414 includes a second latch 2418 at a distal end of the second latch arm 2414 (the end opposite to the body portion 2404). The first latch 2416 and the second latch 2418 are directed upwards in the orientation illustrated in FIG. 14A, i.e., out of the plane of the paper. The first latch 2416 and the second latch 2418 are therefore parallel to each other and protrude in a direction normal to the plane of the body portion 2404 and parallel to the mid-plane 2442 (perpendicular to the needle 2410). As described below, the first latch 2416 and the second latch 2418 are configured to latch onto internal latch points of the first latch opening 2312 and the second latch opening 2314 of the infusion portion 2302, 2320, respectively, during use.

A first tab 2420 is joined to the first latch arm 2412. Similarly a second tab 2422 is joined to the second latch arm 2414. The first tab 2420 and the second tab 2422 extend to overlie over a part of the body portion 2404 and are arranged in a plane parallel to, and spaced from, the plane of the body portion 2404. As shown in FIG. 14B to FIG. 14D, the first tab 2420 is joined to a side of the first latch arm 2412 opposite to the central part 2432 and second latch arm 2414 and extends to partially overlie the body portion 2404. A space 2434 is defined between the first tab 2420 and the body portion 2404. Similarly, the second tab 2422 is joined to a side of the second latch arm 2414 opposite to the central part 2432 and first latch arm 2412 and extends to partially overlie the body portion 2404. A space 2436 is defined between the second tab 2422 and the body portion 2404. The ends of the first tab 2420 and the second tab 2422 are opposing and spaced apart proximate to the central part 2432 of the body portion 2404.

As apparent in FIG. 14B and FIG. 14D, pressing the first tab 2420 towards the body portion 2404 would deflect the first latch arm 2412 at a first flexing point 2438. That is, the first flexing point 2438 would flex or deform to permit movement of the first latch arm 2412. Similarly, pressing the second tab 2422 towards the body portion 2404 would deflect the second latch arm 2414 at a second flexing point 2440. That is, the second flexing point 2440 would flex or deform to permit movement of the second latch arm 2414.

The first flexing point 2438 and the second flexing point 2440 may be formed by a thinner section of the body portion 2404, as illustrated. In particular, the first flexing point 2438 and the second flexing point 2440 join the first latch arm 2412 and the second latch arm 2414 to the body portion 2404 and have a reduced material thickness so define a weak point in the tube connector 2402 that would flex when the first tab 2420 and the second tab 2422 are pressed.

Alternatively, the first flexing point 2438 and the second flexing point 2440 may comprise a different material (e.g., provided by co-moulding), or by an embedded or attached feature such as a spring feature.

Therefore, the first tab 2420 and the second tab 2422 can be pressed towards the body portion 2404 to deflect the first latch arm 2412 and the second latch arm 2414 in the direction opposite to the first latch 2416 and the second latch 2418. Accordingly, the first tab 2420 and the second tab 2422 can be pressed to connect and disconnect the tube connector 2402 and the infusion portion 2302, 2320 as described further below.

As shown in FIG. 14A to FIG. 14D, the tube connector 2402 also includes a first guide arm 2424 and a second guide arm 2426. The first guide arm 2424 and the second guide arm 2426 protrude from the body portion 2404, in particular from the front side 2428 of the body portion 2404. The first guide arm 2424 and the second guide arm 2426 extend parallel to the first latch 2416 and the second latch 2418, and parallel to the needle 2410. The first guide arm 2424 and the second guide arm 2426 are arranged inwardly of the first latch arm 2412 and the second latch arm 2414, closer to the central part 2432 of the body portion 2404. The first guide arm 2424 is adapted to be received in the first guide opening 2316 of the infusion portion 2302, 2320 (see FIG. 13A and FIG. 13B), and the second guide arm 2426 is adapted to be received in the second guide opening 2318 of the infusion portion 2302, 2320 (see FIG. 13A and FIG. 13B). The first guide arm 2424 and the second guide arm 2426, in cooperation with the first guide opening 2316 and the second guide opening 2318 respectively, act to guide connection of the tube connector 2402 and the infusion portion 2302, 2320 and particularly to align the needle 2410 with the fluid connector 2308.

Referring to FIG. 13A, FIG. 13B and FIG. 14A to FIG. 14D, during connection of the tube connector 2402 to the infusion portion 2302, 2320 the first latch arm 2412 is received in the first latch opening 2312 and the second latch arm 2414 is received in the second latch opening 2314. The first guide arm 2424 and the second guide arm 2426 are simultaneously received in the first guide opening 2316 and the second guide opening 2318, respectively. As the tube connector 2402 is pushed onto the infusion portion 2302, 2320 the first latch 2416 and the second latch 2418 will engage corresponding latch features in the first latch opening 2312 and the second latch opening 2314, respectively. Engagement of the first guide opening 2316 and the second guide opening 2318 with the latch features may deflect the first latch arm 2412 and the second latch arm 2414 and further movement will couple the first latch 2416 and the second latch 2418 with the corresponding latch features of the infusion portion 2302, 2320. Additionally or alternatively, the user may press the first tab 2420 and the second tab 2422 towards the body portion 2404 to deflect the first latch arm 2412 and the second latch arm 2414 and facilitate engagement of the first latch 2416 and the second latch 2418 with the corresponding latch features. Meanwhile, the needle 2410 has connected with the fluid connector 2308, for example by piercing a septum of the fluid connector 2308 and forming a fluid connection with the cannula 2310.

The tube connector 2402 can be disconnected from the infusion portion 2302, 2320 by pressing the first tab 2420 and the second tab 2422 towards the body portion 2404 to disengage the first latch 2416 and the second latch 2418 from the corresponding latch features, and pulling the tube connector 2402 away from the infusion portion 2302, 2320.

During connection and disconnection the first tab 2420 and the second tab 2422 advantageously provide improved contact area for a user to grip, and improved leverage for deflecting the first latch arm 2412 and the second latch arm 2414. In particular, the first tab 2420 and the second tab 2422 together define a pressing surface for a user to press on, in particular with a single thumb or finger. The user can therefore press down on the tube connector 2402 to depress the first tab 2420 and the second tab 2422 for connecting and disconnecting the tube connector 2402. This advantageously reduces the force required to deflect the first latch arm 2412 and the second latch arm 2414, and makes the force easier to apply, which may be advantageous for users with impaired movement.

As described above, the tube connector 118, 2212, 2402 is used to connect a connecting tube 116, 2214, 2406 to an infusion portion 104, 2204, 2302, 2320. The tube connector 118, 2212, 2402 has a connecting tube 116, 2214, 2406 attached to it and clips into the infusion portion 104, 2204, 2302, 2320.

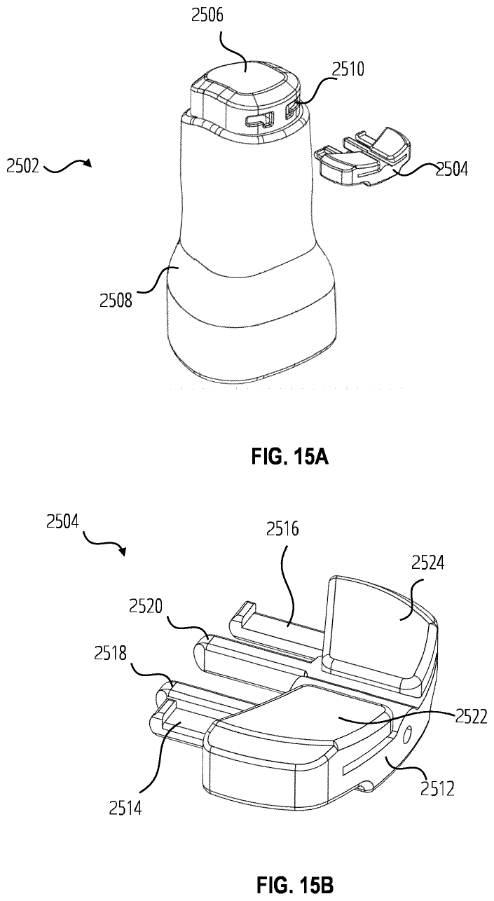

In other examples, as shown in FIG. 15A and FIG. 15B, a similar safety connector 2504 can be used with an insertion device 2502. The insertion device 2502 shown in FIG. 15A is for attaching an infusion portion (e.g., the infusion portion 104, 2204, 2302, 2320) to a user's skin 102 in the manner shown in FIG. 1A, so that the cannula 112 is inserted into a subcutaneous position. The insertion device 2502 includes an insertion device body 2508 that holds the infusion portion 104, 2204, 2302, 2320 and the mounting portion 108 (see FIG. 1A). The infusion portion 104, 2204, 2302, 2320 is held with the cannula directed towards an open end of the insertion device body 2508. A button 2506 is provided at the opposite end and pressing the button 2506 pushes the infusion portion 104, 2204, 2302, 2320 down through the insertion device body 2508, out of the open end, and into contact with the user's skin. In this way the cannula pierces the skin and the mounting portion attaches the infusion portion 104, 2204, 2302, 2320 to the skin.

Insertion devices 2502 are known, for example, from EP2552513B1.

As shown in FIG. 15A, a safety connector 2504 is used in connection portion 2510 to lock the button 2506 in an extended position before use of the insertion device 2502. When the infusion system is provided to a user the infusion portion 104, 2204, 2302, 2320 is provided within the insertion device body 2508 of the insertion device 2502, and the safety connector 2504 is positioned to lock the button 2506. Before use, the user removes the safety connector 2504 from the connection portion 2510, positions the insertion device 2502 on their skin, and pushes the button 2506 to insert the cannula and attach the infusion portion 104, 2204, 2302, 2320 to the skin.

As shown in FIG. 15B, the safety connector 2504 is similar to the tube connector 2402 described above with reference to FIG. 14A to FIG. 14D. The difference between the safety connector 2504 and the 2402 described with reference to FIG. 14A to FIG. 14D is that the safety connector 2504 does not include a tube connection point or a needle or fluid path. However, in order to simplify manufacturing and reduce the number of unique components, the safety connector 2504 may include a tube connection and fluid path which are not used.

The remainder of the safety connector 2504 is as described with reference to FIG. 14A to FIG. 14D above. In particular, the safety connector 2504 includes a body portion 2512 from which extend a first latch arm 2514 and a second latch arm 2516. A first tab 2522 is joined to the first latch arm 2514 and a second tab 2524 is joined to the second latch arm 2516. The first tab 2522 and the second tab 2524 are as described with reference to the tube connector 2402 of FIG. 14A to FIG. 14D and can be pressed to deflect the first latch arm 2514 and the second latch arm 2516. The safety connector 2504 also includes a first guide arm 2518 and a second guide arm 2520.

As shown in FIG. 15A, the insertion device 2502 includes a connection portion 2510 that receives the safety connector 2504. In particular, the connection portion 2510 includes an opening that receives the first latch arm 2514, the second latch arm 2516, the first guide arm 2518 and the second guide arm 2520. When the safety connector 2504 is connected to the connection portion 2510 the first latch arm 2514, second latch arm 2516, first guide arm 2518 and/or the second guide arm 2520 prevents downward movement of the button 2506, thereby preventing accidental or early actuation of the insertion device 2502.

As mentioned above, before using the insertion device 2502 the safety connector 2504 is removed, allowing the button 2506 to be depressed. Therefore, the advantages of the tube connector 2402 described above, in terms of case of handling for removal, apply to the safety connector 2504 also. In addition, it is advantageous to provide the same connector as the safety connector 2504 and as the tube connector 2402 so that the patient does not have to use two different types of connector.

As mentioned above, the tube connector 118, 2212, 2412 can be coupled via the connecting tube 2214 to a pump for administrating a therapeutic substance, for example. However, the tube connector 118, 2212, 2412 can be replaced with a dummy connector or dummy part, which dummy part is shaped to correspond to the tube connector 118, 2212, 2412 but without the connecting tube 2214. In this way, a patient can disconnect the real tube connector 118, 2212, 2412 and replace the real tube connector 118, 2212, 2412 with the dummy part. In this way, the pump for administrating medicines or a therapeutic substances can be disconnected from the patient's infusion set 2202. When the patient is wearing the infusion set 2202 with the dummy part or dummy connector and is in need of medication or a therapeutically substance, the patient can e.g. inject the medication or a therapeutically substance through a part of the 2202 directly into the cannula 2210, for example by using a syringe.

In this way, it is easier for the patient to leave the house for a shorter period of time without the inconvenience of having to bring unmanageable medical equipment such as the above mentioned pump for administrating medicines, but still be able to inject medicines or a therapeutic substance by use of e.g., a small syringe when needed.

Similarly, the safety connector 2504 illustrated in FIG. 15A and FIG. 15B may be used as the dummy part after being removed from the insertion device 2502.

In some examples, the safety connector 2504 may additionally include the fluid path 2408 and the needle 2410 shown in FIG. 14A to FIG. 14D, and a pierceable region, for example a septum, located at the opening of the fluid path 2408. In this way, the septum may allow a patient to administer medicament by injecting medicament through the fluid path 2408 and needle 2410 and into the infusion set 2202.

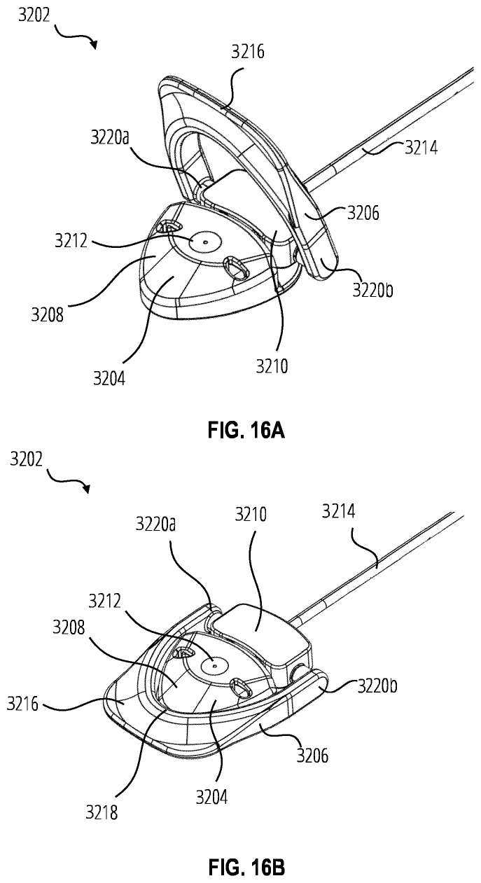

FIG. 16A and FIG. 16B illustrate an example infusion set 3202, with an infusion portion 3204 and a tube connector 3206. In the illustrated examples the tube connector 3206 is connected to the infusion portion 3204. In FIG. 16A the tube connector 3206 is connected to the infusion portion 3204 and in FIG. 16B the tube connector 3206 is connected and locked to the infusion portion 3204.

As illustrated in FIG. 16A and FIG. 16B the infusion portion 3204 has a body portion 3208 that is attachable to a user as described with reference to FIG. 1A to FIG. 1C. A cannula 3212 is provided within the body portion 3208 and the cannula 3212 is subcutaneously positioned when the body portion 3208 is attached to the user. It will be appreciated that the cannula 3212 protrudes from the opposite side of the body portion 3208 to that illustrated, and the top of cannula 3212 is indicated in FIG. 16A and FIG. 16B.

As illustrated, the infusion portion 3204 also includes a tube connecting portion 3210 for connecting with a connecting tube 3214 via the tube connector 3206. The tube connecting portion 3210 and the tube connector 3206 fluidly couple when the tube connector 3206 is connected to the infusion portion 3204. The tube connecting portion 3210 may include a fluid port and/or needle, and the tube connector 3206 may include a fluid port and/or a needle that fluidly connects with the tube connecting portion 3210 as described in further detail hereinafter.

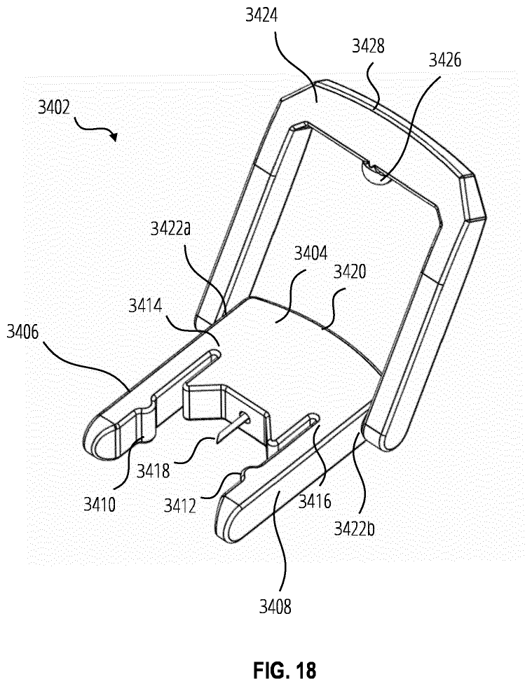

As illustrated, the tube connector 3206 also includes a locking member, in this example a loop member 3216 that is rotatable to lock the tube connector 3206 to the infusion portion 3204. The tube connector 3206 is described in more detail with reference to FIG. 18.

In FIG. 16A the loop member 3216 is shown in an open position, where it is rotated away from the infusion portion 3204. In this position the tube connector 3206 can be connected to the infusion portion 3204 and can be disconnected from the infusion portion 3204.

As shown in FIG. 16B, the loop member 3216 can be rotated towards the infusion portion 3204 so that it surrounds at least a part of the body portion 3208. In particular, the loop member 3216 can surround the body portion 3208 and extend to an opposite side of the body portion 3208 to the tube connecting portion 3210. In this way, the loop member 3216 prevents the tube connector 3206 from being disconnected from the infusion portion 3204. In this position, the loop member 3216 is in a locked position in which the tube connector 3206 is locked onto the infusion portion 3204.

Accordingly, to connect the tube connector 3206 to the infusion portion 3204 the tube connector 3206 is pushed onto the infusion portion 3204 with the loop member 3216 rotated away from the infusion portion 3204 as shown in FIG. 16A. When attached the tube connecting portion 3210 is connected to the connecting tube 3214 through the tube connector 3206. Then, the loop member 3216 is rotated down to surround the body portion 3208 as shown in FIG. 16B.

Advantageously, providing the loop member 3216 may obviate the need for any latches, catches, tines or detents or similar that may increase the insertion force to connect the tube connector 3206 to the infusion portion 3204.

As best shown in FIG. 16B, the loop member 3216 may include a clip portion 3218 that clips over a part of the body portion 3208 to hold the loop member 3216 in the locked position shown in FIG. 16B. In particular, the clip portion 3218 may contact the body portion 3208 in an interference fit, a push-fit, a press-fit or similar so that pushing the loop member 3216 down locks the loop member 3216 to the body portion 3208. A part of the body portion 3208 may be shaped to correspond to the loop member 3216 to facilitate the fit between the body portion 3208 and the loop member 3216. For disconnecting the tube connector 3206 the interference between the loop member 3216 and the body portion 3208 may be overcome by a user to move the loop member 3216 back to the position shown in FIG. 16A.

The clip portion 3218 is illustrated as being in a position on the body portion 3208 opposite to the tube connecting portion 3210, but it will be appreciated that one or more clip portions 3218 may be provided on the loop member 3216, for example on the sides of the body portion 3208.

As shown, the loop member 3216 is rotatably attached to the tube connector 3206 at a hinge 3220a, 3220b. The hinge of this example has two hinges 3220a, 3220b, but it will be appreciated that a single hinge may be provided. The hinge 3220a, 3220b may include a spring, for example a torsion spring or similar, to urge rotation of the loop member 3216 in a particular direction or to a particular position. In some examples, the hinge 3220a, 3220b may include an overcentre mechanism arranged to urge rotation of the loop member 3216 towards either the position shown in FIG. 16A or the position shown in FIG. 16B, or some other rotational position.

In some examples, the hinge(s) 3220a, 3220b may restrict or limit rotation of the loop member 3216 relative to the tube connector 3206. In some examples, the loop member 3216 may be rotatable to a position 180 degrees from the position illustrated in FIG. 16B, so that the loop member 3216 is substantially parallel to the connecting tube 3214. In this position the loop member 3216 may be gripped and pulled for removing the tube connector 3206 from the infusion portion 3204. The loop member 3216 can advantageously be gripped in different ways, and may be easier to grip for a patient with impaired dexterity, allowing the patient to more easily connect and disconnect the tube connector 3206 and the infusion portion 3204.

It will be appreciated that the loop member 3216 of this example is a locking member rotatable to lock the tube connector 3206 to the infusion portion 3204. In some examples, an alternative locking member may not be a loop, and may instead be an arm (e.g., a dog-legged or an L-shaped arm) or other shape that, when rotated, engages the infusion portion 3204 to lock the tube connector 3206 to the infusion portion 3204. In some examples, the infusion portion 3204 may include an opening, for example a notch, to receive a part of the locking member in the locked position. Therefore, the locking member may be a loop member 3216 as shown in FIG. 16A and FIG. 16B, or may be a different shape rotatable member.

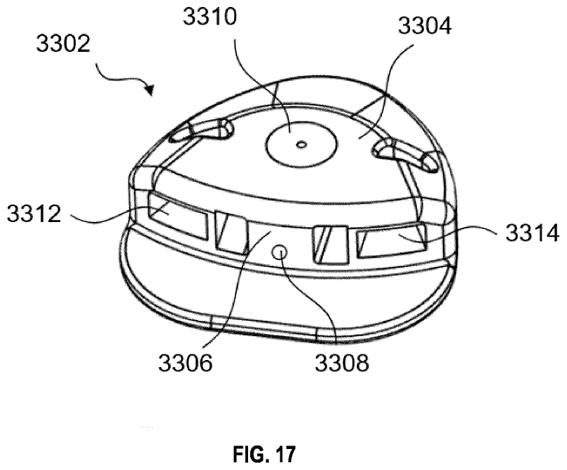

FIG. 17 illustrates an example infusion portion 3302 that may be the infusion portion 3204 described with reference to FIG. 16A. As shown, the infusion portion 3302 includes a body portion 3304 that is attachable to a user as described with reference to FIG. 1A to FIG. 1C, for example by an adhesive patch. A cannula 3310 is provided within the body portion 3304 and the cannula 3310 is subcutaneously positioned when the body portion 3304 is attached to the user. It will be appreciated that the cannula 3310 protrudes from the opposite side of the body portion 3304 to that illustrated, and the top of cannula 3310 is indicated in FIG. 16A. The infusion portion 3302 also includes a tube connecting portion 3306 for connecting with a connecting tube via a tube connector as described with reference to FIG. 18.

The tube connecting portion 3306 includes a fluid connector 3308 for forming a fluid connection with the tube connector and connecting tube shown in FIG. 18. The fluid connector 3308 may include a septum or other seal that is pierced by the tube connector, and allows for resealing the fluid connector 3308 when the tube connector is disconnected from the infusion portion 3302. In other examples the fluid connector 3308 may include a needle protruding from the infusion portion 3302 for forming a fluid connection with a fluid port of the tube connector.

As shown in FIG. 17, the tube connecting portion 3306 includes a first opening 3312 and a second opening 3314 that receive first and second arms of the tube connector described further with reference to FIG. 18. The first opening 3312 and the second opening 3314 may include internal latch portions as described further below.

FIG. 18 illustrates a tube connector 3402 for use with the infusion portion 3302 of FIG. 17. The tube connector 3402 may be the tube connector 3206 described with reference to FIG. 16A and FIG. 16B.

As shown in FIG. 18, the tube connector 3402 includes a connector body 3404. The connector body 3404 includes a tube connection point 3420 where a tube (not shown) is connected. The connector body 3404 also includes a needle 3418 and a fluid path extending between the needle 3418 and the tube connection point 3420. When the tube connector 3402 is connected to an infusion portion the fluid path provides a fluid connection between the tube and the cannula as described previously. In other examples, instead of a needle 3418 the tube connector 3402 may include a connection point for connecting to a needle of the infusion portion.

As shown, the tube connector 3402 includes a locking member, in this example a loop member 3424. The loop member 3424 is rotatably mounted to the connector body 3404 at hinges 3422a, 3422b in a similar manner to as described with reference to FIG. 16A and FIG. 16B. The loop member 3424 is rotatable relative to the connector body 3404 so that the loop member 3424 can be used as a pull ring and/or as a locking mechanism, as described below. The loop member 3424 includes a clip portion 3426 for clipping the loop member 3424 in a locked position, as described further below. The loop member 3424 also includes a finger grip 3428 for easier gripping of the loop member 3424 for lifting it from a locked position, as described further below.

As shown in FIG. 18, the tube connector 3402 includes a first arm 3406 and a second arm 3408. The first arm 3406 and the second arm 3408 extend from the connector body 3404 The first arm 3406 and the second arm 3408 are substantially parallel to each other. The needle 3418 is disposed between the first arm 3406 and the second arm 3408 and directed in substantially the same direction as the first arm 3406 and the second arm 3408. In examples where the needle 3418 includes a needle, the needle extends parallel to the first arm 3406 and the second arm 3408 and between the first arm 3406 and the second arm 3408. As described below, this allows the tube connector 3402 to be connected to the infusion portion by a sideways sliding movement that forms a fluid connection with the needle 3418 while the first arm 3406 and the second arm 3408 couple the tube connector 3402 to the infusion portion.

In the illustrated example the first arm 3406 includes a first latch 3410 and the second arm 3408 includes a second latch 3412. The first latch 3410 is directed inwardly, towards from the second arm 3408 and the second latch 3412 is directed inwardly, towards the first arm 3406. As described below, the first latch 3410 and the second latch 3412 may be configured to latch onto internal latch points of the first opening 3312 and the second opening 3314 of the infusion portion 3302, respectively, during use to connect the tube connector 3402 to the infusion portion 3302.

The first arm 3406 is joined to the connector body 3404 at a first flexing point 3414. The second arm 3408 is joined to the connector body 3404 at a second flexing point 3416. The first flexing point 3414 and the second flexing point 3416 are resiliently deformable such that the first arm 3406 and the second arm 3408 are resiliently deflectable, allowing the first latch 3410 and the second latch 3412 to be latched and unlatched from the infusion portion. The first flexing point 3414 and the second flexing point 3416 may be formed by a thinner section of the connector body 3404, or by a different material (e.g., provided by co-moulding), or by an embedded or attached feature such as a spring feature.

In some alternative examples the first arm 3406 and the second arm 3408 may not include any latches. In such examples, the loop member 3424 may be relied on to hold the tube connector 3402 in connection with the infusion portion 3302.

Referring to FIG. 17 and FIG. 18, during connection of the tube connector 3402 to the infusion portion 3302 the first arm 3406 is received in the first opening 3312 and the second arm 3408 is received in the second opening 3314. During connection the loop member 3424 is rotated away from the infusion portion 3302 as shown in FIG. 18. As the tube connector 3402 is pushed onto the infusion portion 3302 the first latch 3410 (if provided) and the second latch 3412 (if provided) will engage corresponding latch features in the first opening 3312 and the second opening 3314, respectively. Meanwhile, the needle 3418 is connected with the fluid connector 3308. In examples, one of the needle 3418 and the fluid connector 3308 may include a needle, and the other may include a fluid port (e.g., a septum) that is punctured by the needle to form a fluid connection. In this way, the needle 3418 forms a fluid connection with the cannula 3310.

To lock the tube connector 3402 to the infusion portion 3302 the loop member 3424 can be rotated over to surround the body portion 3304, as shown in FIG. 16B. As described with reference to FIG. 16A and FIG. 16B, the loop member 3424 may include a clip portion 3426 for contacting/interfering with the body portion 3304 to clip the loop member 3424 in the locked position.

The tube connector 3402 can be disconnected from the infusion portion 3302 by rotating the loop member 3424 away from the infusion portion 3302 using the finger grip 3428 to overcome any restraining force provided by the clip portion 3426. Then the tube connector 3402 can be pulled away from the infusion portion 3302. If the first latch 3410 and the second latch 3412 are provided then pulling the tube connector 3402 would overcome any latching force, otherwise the tube connector 3402 will slide away from the infusion portion 3302. In some examples the loop member 3424 can be rotated back and provide a pull ring for disconnecting the tube connector 3402 from the infusion portion 3302. Advantageously, only a small force is required to connect or disconnect the tube connector 3402 from the infusion portion 3302, making it easier for user with impaired movement. The loop member 3424 may also be easier to grip, and can be gripped in different ways, making it easier for a patient with impaired dexterity to connect and disconnect the tube connector 3402 and the infusion portion 3302.

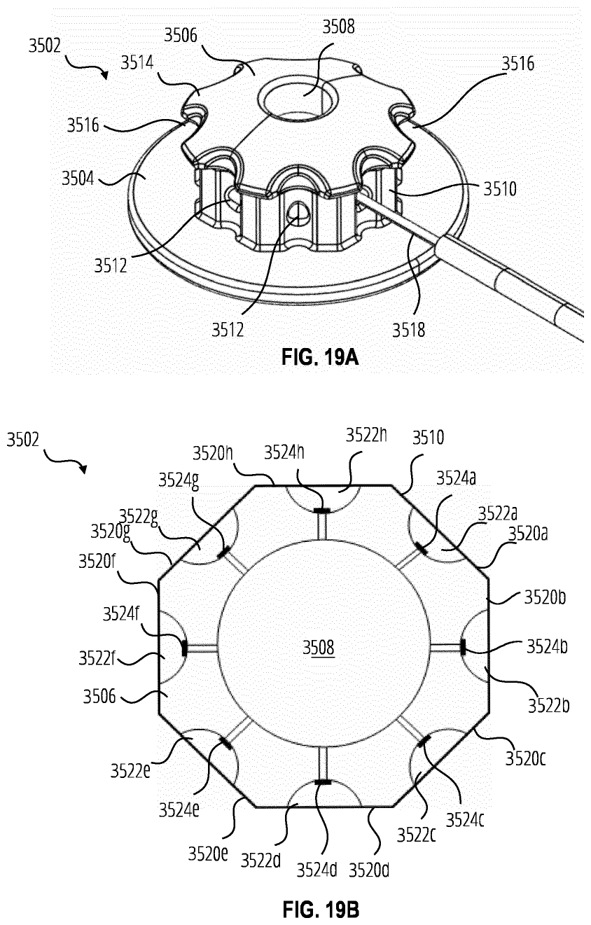

FIG. 19A shows a further example infusion portion 3502 of an infusion set. The infusion portion 3502 includes a base portion 3504 and a spigot 3506 protruding from the base portion 3504. In use, the base portion 3504 is attached to a patient's skin, for example using an adhesive patch as described with reference to FIG. 1A. The infusion portion 3502 includes a cannula in cannula opening 3508. A side wall 3510 of the spigot 3506 includes a plurality of fluid ports 3512. Each fluid port 3512 is in fluid communication with the cannula through the cannula opening 3508, and may include a seal, for example a septum. Accordingly, a needle 3518 of a tube connector may be connected to any of the fluid ports 3512 for fluidly connecting with the cannula through the cannula opening 3508.

The spigot 3506 also includes a flange edge 3514. The flange edge 3514 has a plurality of recesses 3516 formed about its peripheral edge. In this way, the flange edge 3514 has an undulating form. Each fluid port 3512 is in alignment with a recess 3516 of the flange edge 3514. As illustrated, the recesses 3516 may extend through the side wall 3510 as well, as described further below with reference to FIG. 19B.

Accordingly, a tube connector can be attached to the infusion portion 3502 from any of a number of different directions in which the needle 3518 aligns with a fluid port 3512 in the infusion portion 3502. As described below, the shape of the side wall 3510 and/or the flange edge 3514 ensures correct alignment between the tube connector and one of the fluid ports 3512.

FIG. 19B shows a cross-section through the infusion portion 3502, in particular through the spigot 3506 below the flange edge 3514. As shown, the side wall 3510 of the spigot 3506 has a polygonal form. That is, the cross-section of the side wall 3510 is polygonal. In particular, the cross-section of the side wall 3510 has a regular polygonal cross-section with an even number of sides. Specifically, the side wall 3510 has a number of sides 3520a-3520h that is exactly divisible by four. The cross-section of the side wall 3510 is therefore regular and symmetrical, and the sides are arranged in parallel opposing pairs of sides with identical lengths.

In the illustrated example the side wall 3510 has eight sides 3520a-3520h. Each side 3520a-3520h has a recess 3522a-3522h, and a fluid port 3524a-3524h. In other examples, the side wall 3510 may have four sides, or twelve sides, or sixteen sides, each with a fluid port. Each side 3520a-3520h may or may not have a recess as shown in FIG. 19B.

Each fluid port 3524a-3524h is connected to the cannula opening 3508, which is connected to the cannula. In this way, any of the fluid ports 3524a-3524h can be used to connect to the cannula. Each fluid port 3524a-3524h comprises a seal, for example a septum, that can be punctured by a needle as described below.

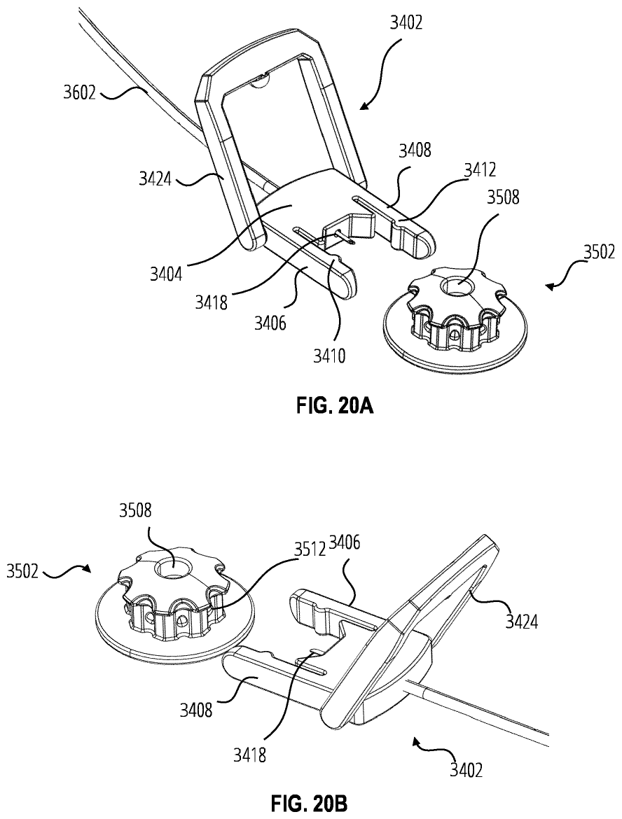

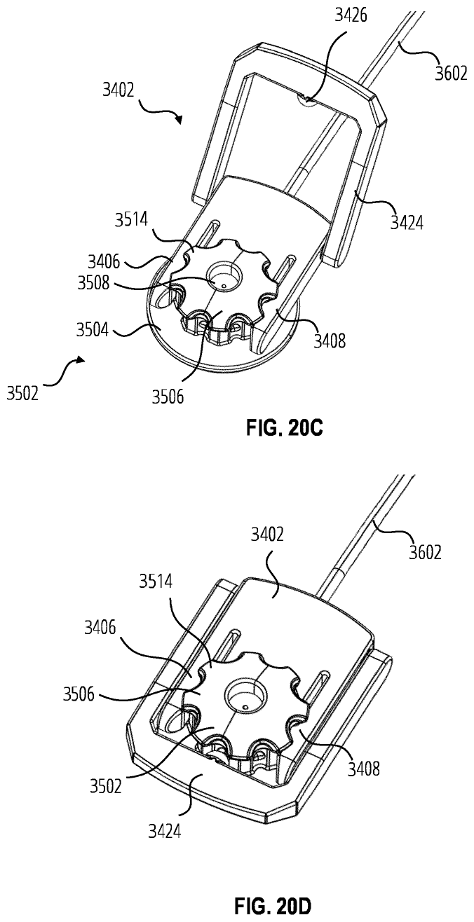

FIG. 20A to FIG. 20D illustrate a tube connector 3402 described with reference to FIG. 18 being connected to the infusion portion 3502 described with reference to FIG. 19A and FIG. 19B. In FIG. 20A to FIG. 20D the tube connector 3402 includes a needle 3418 and a connecting tube 3602. The needle 3418 extends from the connector body 3404 between the first arm 3406 and the second arm 3408. The connecting tube 3602 is connected on the opposite side of the connector body 3404 and is fluidly connected to the needle 3418 through a fluid path in the connector body 3404. When the tube connector 3402 is connected to the infusion portion 3502 the needle 3418 provides a fluid connection between the connecting tube 3602 and the cannula in the cannula opening 3508 as described previously.

As shown in FIG. 20A, before the tube connector 3402 is connected to the infusion portion 3502 the loop member 3424 is rotated away from the infusion portion 3502, into an open position that allows the tube connector 3402 to be slid sideways onto the infusion portion 3502 as described below.

As shown in FIG. 20B, during connection of the tube connector 3402 and the infusion portion 3502 the needle 3418 is aligned with a fluid port 3512 of the infusion portion 3502. The needle 3418 passes into the fluid port 3512 to form a fluid connection between the connecting tube (3602, see FIG. 20A) and the cannula in the cannula opening 3508.

Referring to FIG. 20B and FIG. 20C, as the tube connector 3402 is pushed onto the infusion portion 3502, with the needle 3418 in a fluid port 3512, the first arm 3406 and the second arm 3408 are positioned either side of the spigot 3506 of the infusion portion 3502. The first arm 3406 and the second arm 3408 are positioned between the flange edge 3514 and the base portion (3504, see FIG. 19A).

In particular, the first arm 3406 and the second arm 3408 contact opposite sides of the spigot 3506. The first arm 3406 and the second arm 3408 are spaced to only allow the tube connector 3402 to be slid onto the infusion portion 3502 if the first arm 3406 and the second arm 3408 are parallel to two opposite sides of the spigot 3506 (e.g., side 3520a and side 3520c as shown in FIG. 19B). Due to the regular polygonal cross-section of the spigot 3506 having a number of sides side 3520a-3520h that is exactly divisible by four (see description of FIG. 19B), and the location of the fluid ports 3524a-3524h in each side 3520a-3520h, the tube connector 3402 can only be connected to the infusion portion 3502 when the needle 3418 is aligned with a fluid port 3524a-3524h.

Accordingly, the shape of the infusion portion 3502, in particular the spigot 3506, allows the tube connector 3402 to be connected by a sideways sliding movement from a number of different directions. In the illustrated example, the tube connector 3402 can be connected to the infusion portion 3502 from eight different directions. The shape of the infusion portion 3502 also prevents the tube connector 3402 from being pushed onto the infusion portion 3502 unless there is correct alignment.

Additionally, the tube connector 3402 may include a protrusion (not illustrated) that aligns with, and engages, a recess (3516, see FIG. 19A) of the infusion portion 3502, particularly on the flange edge (3514, see FIG. 19A). This can provide further alignment between the needle 3418 and the fluid port (3512, see FIG. 20B) of the infusion portion 3502.

As shown in FIG. 20C, when the tube connector 3402 is pushed onto the infusion portion 3502 the first arm 3406 and the second arm 3408 are positioned either side of the spigot 3506. In examples where the first arm 3406 includes a first latch 3410 and the second arm 3408 includes a second latch 3412, the first latch 3410 and the second latch 3412 may clip over the side wall (3510, see FIG. 19A) of the spigot 3506 to hold the tube connector 3402 on the infusion portion 3502. In particular, the first latch 3410 and the second latch 3412 may engage opposing recesses (recesses 3522a-3522h, see FIG. 19B) of the infusion portion 3502. However, in some examples the first latch 3410 and the second latch 3412 are omitted.

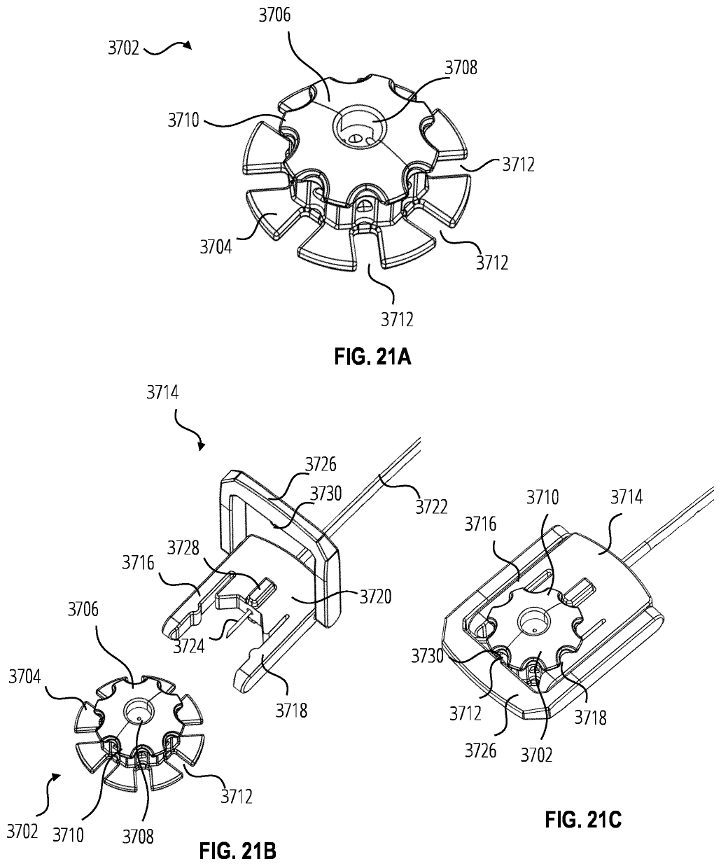

As shown in FIG. 20D, once the needle 3418 is connected with the fluid port (3512, see FIG. 21B), the loop member 3424 can be rotated down to surround the spigot 3506 of the infusion portion 3502. In this position the tube connector 3402 is locked onto the infusion portion 3502 by the loop member 3424.

As described above, the loop member 3424 may have some interference with the spigot 3506, in particular the flange edge 3514, as it is rotated over the spigot 3506. The loop member 3424 may include one or more clip features (3426, see FIG. 20C) that clip over the flange edge 3514 as the loop member 3424 is pushed down over the spigot 3506. This may improve locking by holding the loop member 3424 in the locked position shown in FIG. 7D.

A patient can unlock the tube connector 3402 from the infusion portion 3502 by rotating the loop member 3424 away from the infusion portion 3502. The finger grip 3428 may make it easier for the patient to grip the loop member 3424.

Advantageously, only a small force is required to connect or disconnect the tube connector 3402 from the infusion portion 3502, making it easier for user with impaired movement or dexterity.

FIG. 21A shows a further example infusion portion 3702 of an infusion set. The infusion portion 3702 is the similar to the infusion portion 3502 described with reference to FIG. 19A and FIG. 19B. In particular, the infusion portion 3702 includes a base portion 3704 and a spigot 3706. The spigot 3706 includes a cannula opening 3708 and there are a plurality of fluid ports (not shown in FIG. 21A) in a side wall of the spigot 3706. A flange edge 3710 of the spigot 3706 is provided. The spigot 3706, in particular the side wall, has a regular polygonal cross-section with a number of sides that is exactly divisible by four. The side wall includes a plurality of fluid ports, as described with reference to FIG. 19A and FIG. 19B.

As also shown in FIG. 21A, in this example the base portion 3704 includes a plurality of recesses 3712 spaced about the base portion 3704. The recesses 3712 of the base portion 3704 are aligned with the recesses (3516, see FIG. 19A) of the flange edge 3710, and the recesses (3522a-3522h, see FIG. 19B) of the spigot 3706. The recesses 3712 are thereby also aligned with the fluid ports in the spigot 3706.

As shown in FIG. 21B and FIG. 21C, a tube connector 3714 can be connected to the infusion portion 3702. The tube connector 3714 is similar to the tube connector 3402 described with reference to FIG. 18 above. The tube connector 3714 can be connected to the infusion portion 3702 in the same manner as described in detail with reference to FIG. 20A to FIG. 20D.

In particular, as illustrated, the tube connector 3714 comprises a connector body 3720 with a first arm 3716 and a second arm 3718 extending therefrom. The first arm 3716 and the second arm 3718 are positioned either side of the spigot 3706 of the infusion portion 3702, below the flange edge 3710, when the tube connector 3714 is connected to the infusion portion 3702. As also shown, the tube connector 3714 includes a connecting tube 3722 that is in communication with a needle 3724 that forms a fluid connection with a cannula in the cannula opening 3708 when the tube connector 3714 is connected to the infusion portion 3702. A rotatable locking member, in particular a loop member 3726, is provided for pulling and for locking the tube connector 3714 to the infusion portion 3702 as described above.

As illustrated in FIG. 21B and FIG. 21C, the tube connector 3714 also includes a guide 3728. In this example the guide 3728 is a protrusion. The guide 3728 is disposed on the connector body 3720 and is proximate to the needle 3724. As shown in FIG. 21B and FIG. 21C, when the tube connector 3714 is connected to the infusion portion 3702 the guide 3728 is aligned with one of the recesses 3712 of the flange edge 3710. In this way, the guide 3728 cooperates with a recess 3712 to ensure proper alignment when the tube connector 3714 is attached to the infusion portion 3702.

In a similar manner, the guide 3728 may extend from the opposite side of the connector body 3720 to that illustrated, so that the guide 3728 may additionally or alternative engage a recess 3712 of the base portion 3704 to ensure alignment in the same way.

Accordingly, the tube connector 3714 can be attached to the infusion portion 3702 from a number of different directions in which the needle 3724 aligns with a fluid port (3512, see FIG. 19A) in the infusion portion 3702.

As also shown in FIG. 21B and FIG. 21C, the loop member 3726 may include a clip 3730 that interacts with the infusion portion 3702 to clip the loop member 3726 into the closed position shown in FIG. 21C, as previously described. In this example the clip 3730 is located centrally in the loop member 3726 and arranged to clip into a recess 3712 of the spigot 3706 when the loop member 3726 is in the closed position shown in FIG. 21C.

As described above, the tube connector 118, 3206, 3402, 3714 is used to connect a connecting tube 116, 3214, 3602, 3722 to an infusion portion 104, 3204, 3302, 3502, 3702. The tube connector 118, 3206, 3402, 3714 has a connecting tube 116, 3214, 3602, 3722 attached to it and clips into the infusion portion 104, 3204, 3302, 3502, 3702.

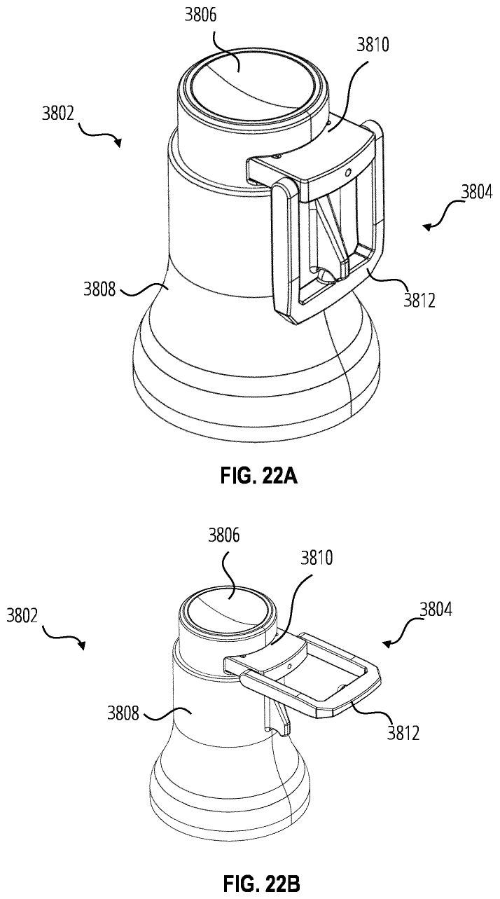

In other examples, as shown in FIG. 22A, a connector similar to the tube connector 118, 3206, 3402, 3714 described above may be provided as a safety connector 3804. The safety connector 3804 can be used with an insertion device 3802. The insertion device 3802 shown in FIG. 22A is for attaching an infusion portion (e.g., the infusion portion 104, 3204, 3302, 3502, 3702) to a user's skin 102 in the manner shown in FIG. 1A, so that the cannula 112 is inserted into a subcutaneous position. The insertion device 3802 includes an insertion device body 3808 that holds the infusion portion 104, 3204, 3302, 3502, 3702 and the mounting portion 108 (see FIG. 1A). The infusion portion 104, 3204, 3302, 3502, 3702 is held with the cannula directed towards an open end of the insertion device body 3808. A button 3806 is provided at the opposite end and pressing the button 3806 pushes the infusion portion 104, 3204, 3302, 3502, 3702 down through the insertion device body 3808, out of the open end, and into contact with the user's skin. In this way the cannula pierces the skin and the mounting portion attaches the infusion portion 104, 3204, 3302, 3502, 3702 to the skin.

Suitable insertion devices 3802 are known, for example, from EP2552513B1.

As shown in FIG. 22A, a safety connector 3804 is used in connection portion 3810 to lock the button 3806 in an extended position before use of the insertion device 3802. When the infusion system is provided to a user the infusion portion 104, 3204, 3302, 3502, 3702 is provided within the insertion device body 3808 of the insertion device 3802, and the safety connector 3804 is positioned to lock the button 3806. The safety connector 3804 is substantially the same as the tube connector 3402 described with reference to FIG. 20A to FIG. 20D, or it may be substantially the same as the tube connector 3714 described with reference to FIG. 21B and FIG. 21C. However, the safety connector 3804 does not have a tube connected thereto. In this position the loop member 3812 of the safety connector 3804 is folded against the insertion device body 3808.

Before use, the user removes the safety connector 3804 from the connection portion 3810 by rotating the loop member 3812 upwards and using the loop member 3812 as a pull ring to pull the safety connector 3804 from the connection portion 3810. The user can then position the insertion device 3802 on their skin, and push the button 3806 to insert the cannula and attach the infusion portion 104, 3204, 3302, 3502, 3702 to the skin.

In particular, as shown in FIG. 22B the loop member 3812 can be rotated away from the insertion device body 3808 so that it can be gripped for pulling the safety connector 3804 out of the connection portion 3810 and away from the insertion device 3802. After removal of the safety connector 3804 the button 3806 can be depressed to operate the insertion device 3802.

Therefore, the advantages of the tube connector 118, 3206, 3402, 3402, 3714 described above, in terms of case of handling for removal, apply to the safety connector 3804 also. In addition, it is advantageous to provide the same connector as the safety connector 3804 and as the tube connector 118, 3206, 3402, 3402, 3714 so that the patient does not have to use two different types of connector. In particular, a patient with impaired dexterity may find it easier to grip the loop member 3812, either by using it as a pull ring or by gripping opposite sides of the loop member 3812.

As mentioned above, the tube connector 118, 3206, 3402, 3714 can be coupled via a connecting tube 116, 3214, 3602, 3722 to a pump for administrating a therapeutic substance, for example. However, the tube connector 118, 3206, 3402, 3714 can be replaced with a dummy connector or dummy part, which dummy part is shaped to correspond to the tube connector 118, 3206, 3402, 3714 but without the connecting tube 116, 3214, 3602, 3722. In this way, a patient can disconnect the real tube connector 118, 3206, 3402, 3714 and replace the real tube connector 118, 3206, 3402, 3714 with the dummy part. In this way, the pump for administrating medicines or a therapeutic substances can be disconnected from the patient's infusion set 3202. When the patient is wearing the infusion set 3202 with the dummy part or dummy connector and is in need of medication or a therapeutically substance, the patient can e.g. inject the medication or a therapeutically substance through a part of the infusion set 3202 directly into the cannula 3212, for example by using a syringe.

In this way, it is easier for the patient to leave the house for a shorter period of time without the inconvenience of having to bring unmanageable medical equipment such as the above mentioned pump for administrating medicines, but still be able to inject medicines or a therapeutic substance by use of e.g., a small syringe when needed.

The safety connector 3804 illustrated in FIG. 22A may be used as the dummy part after being removed from the insertion device 3802.

In some examples, the safety connector 3804 may fluidly couple to the infusion portion 104, 3204, 3302, 3502, 3702 and may include a pierceable region, for example a septum, provided to allow a patient to administer medicament by injecting medicament through the pierceable region and into the infusion set 3202.

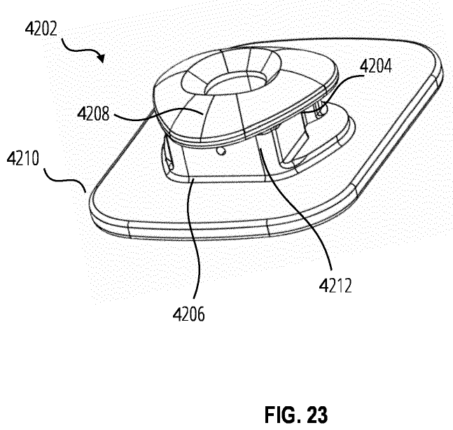

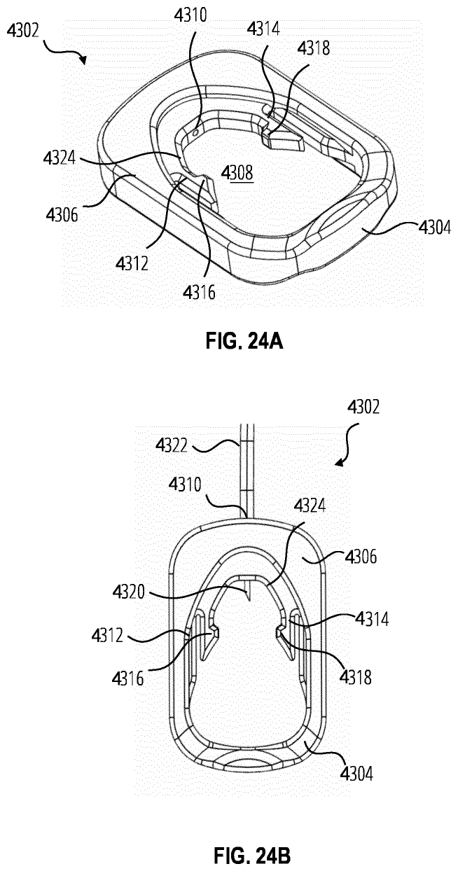

FIG. 23 illustrates an example infusion portion 4202. As shown, the infusion portion 4202 includes a spigot 4204 and a base portion 4210 that is attachable to a user as described with reference to FIG. 1A to FIG. 1C, for example by an adhesive patch. The spigot 4204 protrudes from the base portion 4210. A cannula 4208 is provided within the spigot 4204 and the cannula 4208 is subcutaneously positioned when the spigot 4204 is attached to the user. It will be appreciated that the cannula 4208 protrudes from the opposite side of the spigot 4204 to that illustrated, and the top of cannula 4208 is indicated in FIG. 23. The infusion portion 4202 also includes a fluid port 4206 for connecting with a connecting tube via a tube connector as described with reference to FIG. 24B.

The infusion portion 4202 includes a fluid port 4206 for connecting with the tube connector and connecting tube as described further hereinafter. The fluid port 4206 may include a septum or other seal that is pierced by the tube connector, and allows for resealing the fluid port 4206 when the tube connector is disconnected from the infusion portion 4202. In other examples the fluid port 4206 may include a needle protruding from the infusion portion 4202 for forming a fluid connection with a fluid port of the tube connector.

As shown in FIG. 23, the spigot 4204 has a generally triangular shape. The fluid port 4206 is positioned at a point of the triangular spigot 4204. The spigot 4204 includes a flange edge 4212 that overhangs from a side wall of the base portion 4504.

FIG. 24A and FIG. 24B show a tube connector 4302 for connecting with the infusion portion 4202 illustrated in FIG. 23. The tube connector 4302 is shaped as a loop and includes a second loop portion 4306 and a first loop portion 4304. The second loop portion 4306 and the first loop portion 4304 together define a closed loop. An opening 4308 is defined between the second loop portion 4306 and the first loop portion 4304. The first loop portion 4304 is larger than the second loop portion 4306. The second loop portion 4306 includes a side with a profile 4324. The profile 4324 is opposite to the second loop portion 4306 and is shaped to match one side of the spigot 4204 of the infusion portion 4202 as described further below.

A tube connection 4310 is formed at one end, in this example in the second loop portion 4306. The tube connection 4310 forms a fluid connection with the fluid port 4206 of the infusion portion 4202 shown in FIG. 23. The tube connection 4310 is also connected (or connectable) to a connecting tube 4322, as shown in FIG. 24B. As also shown in FIG. 24B, the tube connector 4302 may include a needle 4320 protruding from the tube connection 4310 into the second loop portion 4306. The needle 4320 connects with the fluid port 4206 of the infusion portion 4202 shown in FIG. 23 as described further below.

As shown, a first arm 4312 and a second arm 4314 are provided within the second loop portion 4306. The first arm 4312 and the second arm 4314 are arranged to engage the infusion portion 4202 when the tube connector 4302 is connected to the infusion portion 4202 as described further below. The first arm 4312 and the second arm 4314 are resiliently attached to the second loop portion 4306 so they can be deflected outwardly as the tube connector 4302 is connected to the infusion portion 4202.

The first arm 4312 may include a first latch 4316 and the second arm 4314 may include a second latch 4318. The first latch 4316 and the second latch 4318 may latch onto the infusion portion 4202 when the tube connector 4302 is connected to the infusion portion 4202.

The first loop portion 4304 is larger than the second loop portion 4306. In particular, the opening within the first loop portion 4304 is larger than the opening within the second loop portion 4306. This is due to the profiles of the second loop portion 4306 and the first loop portion 4304 and also due to the first arm 4312 and the second arm 4314 in the second loop portion 4306.

As shown in FIG. 24B, the tube connector 4302 includes a needle 4320 protruding from the tube connection 4310 into the second loop portion 4306. A connecting tube 4322 is connected to an opposite side of the tube connection 4310, extending away from the second loop portion 4306.

In other examples, the tube connection 4310 may include an opening, optionally with a seal or septum, that is engaged by a needle or similar from the infusion portion 4202.

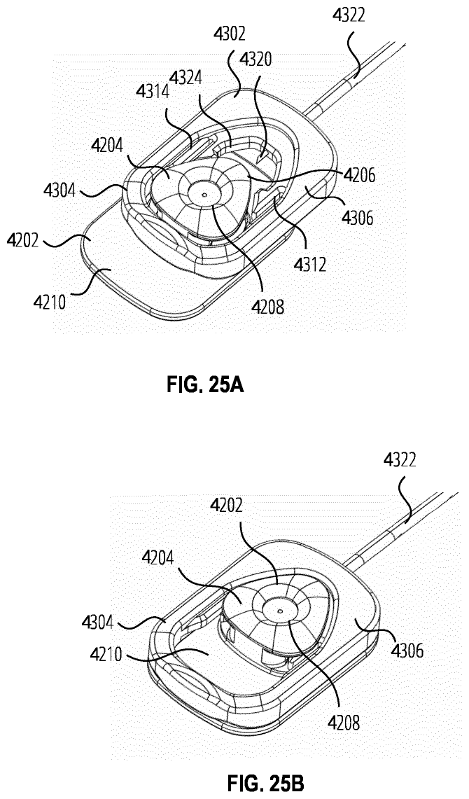

FIG. 25A and FIG. 25B illustrate connection of the tube connector 4302 of FIG. 24B to the infusion portion 4202 of FIG. 23. As shown in FIG. 25A, in an initial stage of connection the tube connector 4302 is placed over the infusion portion 4202 so that the spigot 4204 is positioned within the first loop portion 4304. As described above, the first loop portion 4304 is larger than the second loop portion 4306. The first loop portion 4304 is sized so that the tube connector 4302 can be positioned with the spigot 4204 in alignment with the first loop portion 4304. The second loop portion 4306 is smaller and sized so that the spigot 4204 can not be positioned within the second loop portion 4306 in the same way. In particular, the size of the opening of the second loop portion 4306, and the first arm 4312 and the second arm 4314, would prevent the spigot 4204 from being pushed axially into the second loop portion 4306.

In the position shown in FIG. 25A the tube connector 4302, in particular the needle 4320, faces the fluid port 4206. The first arm 4312 and the second arm 4314 are spaced from the spigot 4204.

At least a part of the first loop portion 4304, particularly the part opposite the second loop portion 4306, is shaped to match the spigot 4204. In particular, the part of the first loop portion 4304 opposite to the second loop portion 4306 is shaped to match a profile of the side wall of the spigot 4204. This can help to ensure that the infusion portion 4202 (particularly the fluid port 4206) and the tube connector 4302 (particularly the tube connection 4310 and needle 4320) are in alignment in the position shown in FIG. 25A.

As shown in FIG. 25B, from the position shown in FIG. 25A the tube connector 4302 is slidable so that the spigot 4204 moves from the first loop portion 4304 into the second loop portion 4306 (through the opening 4308, see FIG. 24A) to the position shown in FIG. 25B. At the same time, the needle 4320 engages the fluid port 4206 to form a fluid connection between the connecting tube 4322 and the cannula 4208.

As the tube connector 4302 is slid, the first arm 4312 and the second arm 4314 engage the spigot 4204 to ensure alignment of the tube connector 4302 and the fluid port 4206. The first arm 4312 and the second arm 4314 may additionally grip or clip onto the spigot 4204 to form a connection between the tube connector 4302 and the infusion portion 4202. In some examples, the first latch 4316 and the second latch 4318 latch onto the spigot 4204 so that the tube connector 4302 is latched onto the infusion portion 4202. In particular, the first arm 4312 and the second arm 4314 can be deflected outwardly by contact with the spigot 4204 and the first latch 4316 and the second latch 4318 can clip into recesses in the spigot 4204, or can clip around an opposing surface of the spigot 4204.

Advantageously, the tube connector 4302 can be easily gripped, particularly by someone with impaired dexterity. In addition, a patient can use the spigot 4204 to lever the tube connector 4302 through the sliding motion described above, meaning that lower overall force is needed to connect the tube connector 4302 to the infusion portion 4202. Similarly, to disconnect the tube connector 4302 from the infusion portion 4202 a patient can grip/push the first loop portion 4304 and may lever against the spigot 4204, making it easier for someone with impaired dexterity to disconnect the tube connector 4302.

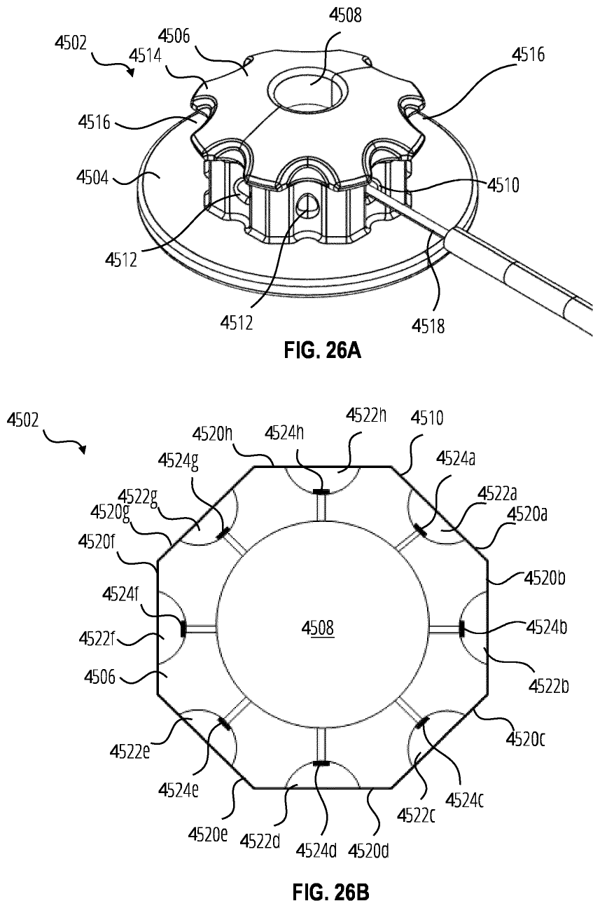

FIG. 26A shows another example infusion portion 4502 of an infusion set. The infusion portion 4502 includes a base portion 4504 and a spigot 4506 protruding from the base portion 4504. In use, the base portion 4504 is attached to a patient's skin, for example using an adhesive patch as described with reference to FIG. 1A. The infusion portion 4502 includes a cannula 4508. A side wall 4510 of the spigot 4506 includes a plurality of fluid ports 4512. Each fluid port 4512 is in fluid communication with the cannula 4508, and may include a seal, for example a septum. Accordingly, a needle 4518 of a tube connector may be connected to any of the fluid ports 4512 for fluidly connecting with the cannula 4508.

The spigot 4506 also includes a flange edge 4514. The flange edge 4514 overhangs the side wall 4510 opposite to the base portion 4504. The flange edge 4514 has a plurality of recesses 4516 formed about its peripheral edge. In this way, the flange edge 4514 has an undulating form. Each fluid port 4512 is in alignment with a recess 4516 of the flange edge 4514. As illustrated, the recesses 4516 may extend through the side wall 4510 as well, as described further below.

Accordingly, a tube connector can be attached to the infusion portion 4502 from any of a number of different directions in which the needle 4518 aligns with a fluid port 4512 in the infusion portion 4502. As described below, the shape of the side wall 4510 and/or the flange edge 4514 can ensure correct alignment between the tube connector and one of the fluid ports 4512.

FIG. 26B shows a cross-section through the infusion portion 4502, in particular through the spigot 4506 below the flange edge 4514. As shown, the side wall 4510 of the spigot 4506 has a polygonal form. That is, the cross-section of the side wall 4510 is polygonal. In particular, the cross-section of the side wall 4510 has a regular polygonal cross-section with an even number of sides. Specifically, the side wall 4510 has a number of sides 4520a-4520h that is exactly divisible by four. The cross-section of the side wall 4510 is therefore regular and symmetrical, and the sides are arranged in parallel opposing pairs of sides with identical lengths.

In the illustrated example the side wall 4510 has eight sides 4520a-4520h. Each side 4520a-4520h has a recess 4522a-4522h, and a fluid port 4524a-4524h. In other examples, the side wall 4510 may have four sides, or twelve sides, or sixteen sides, each with a fluid port. Each side 4520a-4520h may or may not have a recess 4522a-4522h as shown in FIG. 26B.

Each fluid port 4524a-4524h is connected to the cannula 4508. In this way, any of the fluid ports 4524a-4524h can be used to connect to the cannula 4508. Each fluid port 4524a-4524h comprises a seal, for example a septum, that can be punctured by a needle as described below.

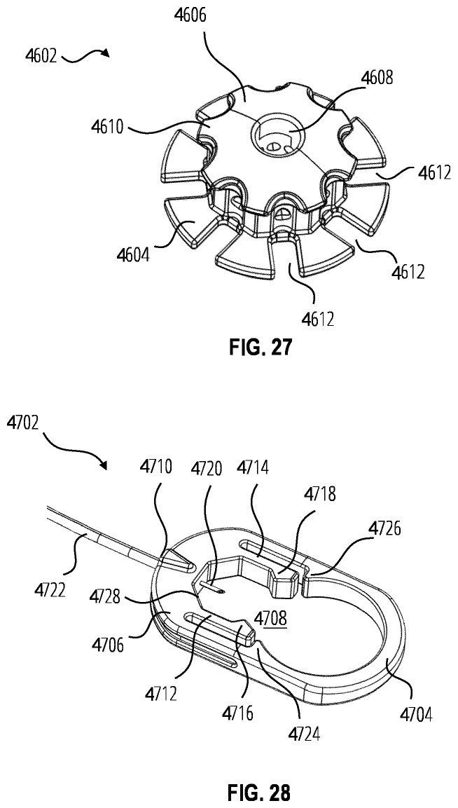

FIG. 27 shows a further example infusion portion 4602 of an infusion set. The infusion portion 4602 is the similar to the infusion portion 4502 described with reference to FIG. 26A and FIG. 26B. In particular, the infusion portion 4602 includes a base portion 4604 and a spigot 4606. The spigot 4606 includes a cannula 4608 and there are a plurality of fluid ports (not shown in FIG. 27) in a side wall of the spigot 4606. A flange edge 4610 of the spigot 4606 is provided. The spigot 4606, in particular the side wall, has a regular polygonal cross-section with a number of sides that is exactly divisible by four as shown in FIG. 26B.

As also shown in FIG. 27, in this example the base portion 4604 includes a plurality of recesses 4612 spaced about the base portion 4604. The recesses 4612 of the base portion 4604 are aligned with the recesses (4516, see FIG. 26A) of the flange edge 4610, and the recesses (4522a-4522h, see FIG. 26B) of the spigot 4606. The recesses 4612 are thereby also aligned with the fluid ports in the spigot 4606.

FIG. 28 illustrates a further example tube connector 4702 for use with the infusion portion 4502 illustrated in FIG. 26A and FIG. 26B and/or the infusion portion 4602 illustrated in FIG. 27.

In this example, the tube connector 4702 is shaped as a loop and includes a first loop portion 4704 and a second loop portion 4706. The second loop portion 4706 and the first loop portion 4704 together define a closed loop. An opening 4708 is defined between the second loop portion 4706 and the first loop portion 4704. The first loop portion 4704 is larger than the second loop portion 4706. The second loop portion 4706 includes a side with a profile 4728. The profile 4728 is opposite to the first loop portion 4704 and is shaped to match the spigot 4506, 4606 of the infusion portion 4502, 4602 as described further below.

As shown, a first arm 4712 and a second arm 4714 are arranged within the second loop portion 4706, on opposite sides. The first arm 4712 and the second arm 4714 are arranged to engage the infusion portion 4502, 4602 as described further below. The first arm 4712 and the second arm 4714 are connected to the second loop portion 4706 at flexing points so that the first arm 4712 and the second arm 4714 are resiliently deflectable. The first arm 4712 includes a first latch 4716 facing the second arm 4714, and the second arm 4714 includes a second latch 4718 facing the first arm 4712.

The tube connector 4702 also includes a fluid connection 4710 for attaching a connecting tube 4722. The tube connector 4702 also includes a needle 4720 extending from the fluid connection 4710 into the second loop portion 4706. The needle 4720 forms a fluid connection with the infusion portion 4502, 4602 when attached, as described further below. The connecting tube 4722 is fluidly connected to the needle 4720 via the fluid connection 4710.

The first loop portion 4704 is larger than the second loop portion 4706. In particular, the opening within the first loop portion 4704 is larger than the opening within the second loop portion 4706. This is due to the profiles of the second loop portion 4706 and the first loop portion 4704 and also due to the first arm 4712 and the second arm 4714 in the second loop portion 4706.

In some examples, the tube connector 4702 may include a protrusion arranged to engage one or more of the recesses (4516, see FIG. 26A) in the flange edge (4514, see FIG. 26A) of the infusion portion 4502. This can ensure alignment between the needle 4720 and a fluid port (4512, see FIG. 26A). Similarly, the tube connector 4702 may include a protrusion arranged to engage one or more of the recesses (4612, see FIG. 27) in the base portion (4604, see FIG. 27) of the infusion portion 4602. This can ensure alignment between the needle 4720 and a fluid port of the infusion portion 4602.

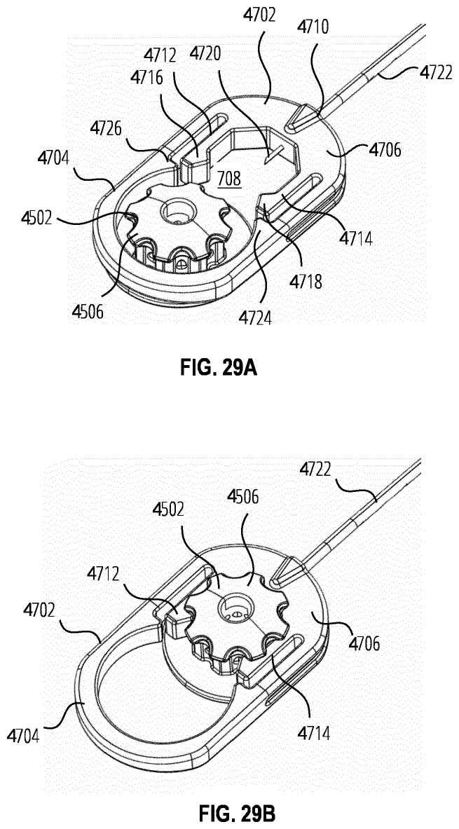

FIG. 29A and FIG. 29B illustrate connection of the tube connector 4702 of FIG. 28 to the infusion portion 4502 of FIG. 26A and FIG. 26B. It will be understood that the tube connector 4702 can be connected to the infusion portion 4602 of FIG. 27 in the same manner as described below with reference to FIG. 29A and FIG. 29B.

As shown in FIG. 29A, in an initial stage of connection the tube connector 4702 is placed over the infusion portion 4502 so that the spigot 4506 is positioned within the first loop portion 4704. The first loop portion 4704 is larger than the second loop portion 4706 and is sized so that the infusion portion 4502 can be positioned with the spigot 4506 within the opening of the first loop portion 4704. The second loop portion 4706 is smaller and is sized so that the spigot 4506 cannot be moved axially into the second loop portion 4706. In particular, the size of the second loop portion 4706, and the first arm 4712 and the second arm 4714, prevent the spigot 4506 from moving axially into the second loop portion 4706.

In the position shown in FIG. 29A the needle 4720 faces the fluid connecting portion spigot 4506 and one of the fluid ports (4512, see FIG. 26A). The first arm 4712 and the second arm 4714 are spaced from the spigot 4506. The protrusion 4724 and the protrusion 4726 are also spaced from the spigot 4506.

At least a part of the first loop portion 4704, particularly the part opposite the second loop portion 4706, is shaped to match the spigot 4506. In particular, the first loop portion 4704 is arcuate (partial circle) that matches the generally cylindrical form of the spigot 4506. This can help to ensure that the infusion portion 4502 and the tube connector 4702 are in alignment, particularly that the needle 4720 is aligned with a fluid port (4512, see FIG. 26A).

As shown in FIG. 29B, from the position shown in FIG. 29A the tube connector 4702 is slidable so that the spigot 4506 moves from the first loop portion 4704 into the second loop portion 4706 (through the opening 4708, see FIG. 28). At the same time, the needle 4720 engages one of the fluid ports (4512, see FIG. 26A) to form a fluid connection between the connecting tube 4722 and the cannula (4508, see FIG. 26A). It will be appreciated that the tube connector 4702 can be connected to any of the fluid ports (4512, see FIG. 26A) of the infusion portion 4502, so the tube connector 4702 can be connected to the infusion portion 4502 in a number of different orientations. This advantageously allows the patient to choose which orientation would be easiest for them based on the position of the infusion portion 4502 on their body and their ability to move their arm/hand to push the tube connector 4702 onto the infusion portion 4502.

As the tube connector 4702 is slid, the protrusion 4724 and the protrusion 4726 guide the spigot 4506 through the opening 4708 and into the second loop portion 4706. A spacing between the protrusion 4724 and the protrusion 4726 may only permit passage of the spigot 4506 through the opening 4708 if the direction of movement is parallel with two opposing sides (e.g., sides 4520a and 4520c, see FIG. 26B). This can ensure alignment of the needle 4720 with a fluid port (4512, see FIG. 26A) of the infusion portion 4502.

As the tube connector 4702 is slid onto the infusion portion 4502 the first arm 4712 and the second arm 4714 engage the spigot 4506. The first arm 4712 and the second arm 4714 may also ensure alignment of the tube connector 4702 and the fluid port (4512, see FIG. 26A). In particular, the first arm 4712 and the second arm 4714 engage opposite sides (e.g., sides 4520a, 4520c, see FIG. 26B) of the side wall (4510, see FIG. 26B) and prevent movement of the tube connector 4702 towards the infusion portion 4502 unless the first arm 4712 and the second arm 4714 are parallel to opposite sides (e.g., sides 4520a, 4520e) of the side wall (4510, see FIG. 26B). In this way, the tube connector 4702 can only be slid into connection with the infusion portion 4502 if the needle 4720 is aligned with a fluid port (4512, see FIG. 26A) of the infusion portion 4502.

The first arm 4712 and the second arm 4714 may additionally grip or clip onto the spigot 4506 to form a connection between the tube connector 4702 and the infusion portion 4502. In some examples, the first latch 4716 and the second latch 4718 latch onto the spigot 4506 so that the tube connector 4702 is latched onto the infusion portion 4502. In particular, the first arm 4712 and the second arm 4714 can be deflected outwardly by contact with the spigot 4506 and the first latch 4716 and the second latch 4718 can clip into recesses (recess 4522a-4522h, see FIG. 26B) in the spigot 4506, or can clip around an opposing side of the spigot 4506.

Advantageously, the tube connector 4702 can be easily gripped, particularly for someone with impaired dexterity. In addition, a patient can use the spigot 4506 to lever the tube connector 4702 through the sliding motion described above, meaning that lower overall force is needed to connect the tube connector 4702 to the infusion portion 4502. Similarly, to disconnect the tube connector 4702 from the infusion portion 4502 a patient can grip/push the first loop portion 4704 and may lever against the spigot 4506, making it easier for someone with impaired dexterity to disconnect the tube connector 4702.

As described above, the tube connector 118, 4302, 4702 is used to connect a connecting tube 116, 4322, 4722 to an infusion portion 104, 4202, 4502, 4602. The tube connector 118, 4302, 4702 has a connecting tube 116, 4322, 4722 attached to it and clips into the infusion portion 104, 4202, 4502, 4602.

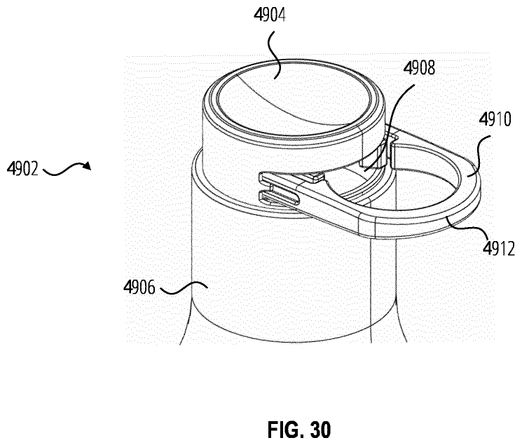

In other examples, as shown in FIG. 30, a safety connector 4910 similar to the tube connector 118, 4302, 4702 described above may be provided for an insertion device 4902. The insertion device 4902 shown in FIG. 30 is for attaching an infusion portion (e.g., the infusion portion 104, 4202, 4502, 4602) to a user's skin 102 in the manner shown in FIG. 1A, so that the cannula 112 is inserted into a subcutaneous position. The insertion device 4902 includes an insertion device body 4906 that holds the infusion portion 104, 4202, 4502, 4602 and the mounting portion 108 (see FIG. 1A). The infusion portion 104, 4202, 4502, 4602 is held with the cannula directed towards an open end of the insertion device body 4906. A button 4904 is provided at the opposite end and pressing the button 4904 pushes the infusion portion 104, 4202, 4502, 4602 down through the insertion device body 4906, out of the open end, and into contact with the user's skin. In this way the cannula pierces the skin and the mounting portion attaches the infusion portion 104, 4202, 4502, 4602 to the skin.

Suitable insertion devices 4902 are known, for example, from EP2552513B1.

As shown in FIG. 30, a safety connector 4910 is used in connection opening 4908 to lock the button 4904 in an extended position before use of the insertion device 4902. When the infusion system is provided to a user the infusion portion 104, 4202, 4502, 4602 is provided within the insertion device body 4906 of the insertion device 4902, and the safety connector 4910 is positioned to lock the button 4904. The safety connector 4910 is substantially the same as the tube connector 4302 described with reference to FIG. 24A and FIG. 24B, or it may be substantially the same as the tube connector 4702 described with reference to FIG. 28. However, the safety connector 4910 does not have a connecting tube or a needle. The safety connector 4910 includes a first loop portion 4912 and a second loop portion (not shown in FIG. 30). When the safety connector 4910 is received in the connection opening 4908, as illustrated, the second loop portion is received within the connection opening 4908 and the first loop portion 4912 protrudes from the connection opening 4908.

Before use, the user removes the safety connector 4910 from the connection opening 4908 by pulling the first loop portion 4912. The first loop portion 4912 advantageously provides a pull ring to pull the safety connector 4910 from the connection opening 4908. The user can then position the insertion device 4902 on their skin, and push the button 4904 to insert the cannula and attach the infusion portion 104, 4202, 4502, 4602 to the skin.

Advantageously, the first loop portion 4912 can be easily gripped, either between finger and thumb or by placing a finger or thumb through the first loop portion 4912, making it easier for a patient with impaired dexterity to remove the safety connector 4910 from the insertion device 4902.

As mentioned above, the tube connector 118, 4302, 4702 can be coupled via a connecting tube 116, 4322, 4722 to a pump for administrating a therapeutic substance, for example. However, the tube connector 118, 4302, 4702 can be replaced with a dummy connector or dummy part, which dummy part is shaped to correspond to the tube connector 118, 4302, 4702 but without the connecting tube 116, 4322, 4722 and needle 4320, 4720. In this way, a patient can disconnect the real tube connector 118, 4302, 4702 and replace the real tube connector 118, 4302, 4702 with the dummy part. In this way, the pump for administrating medicines or a therapeutic substances can be disconnected from the patient's infusion portion 104, 4202, 4502, 4602. The dummy part can seal the infusion portion 104, 4202, 4502, 4602, for example during washing. When the patient is wearing the infusion portion 104, 4202, 4502, 4602 with the dummy part or dummy connector and is in need of medication or a therapeutically substance, the patient can e.g. inject the medication or a therapeutically substance through a part of the infusion portion 104, 4202, 4502, 4602 directly into the cannula, for example by using a syringe.

In this way, it is easier for the patient to leave the house for a shorter period of time without the inconvenience of having to bring unmanageable medical equipment such as the above mentioned pump for administrating medicines, but still be able to inject medicines or a therapeutic substance by use of e.g., a small syringe when needed.

Similarly, the safety connector 4910 illustrated in FIG. 30 may be used as the dummy part after being removed from the insertion device 4902.

In some examples, the safety connector 4910 may fluidly couple to the infusion portion 104, 4202, 4502, 4602 and may include a pierceable region, for example a septum, provided to allow a patient to administer medicament by injecting medicament through the pierceable region and into the infusion portion 104, 4202, 4502, 4602.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. A tube connector for an infusion set, the tube connector comprising:
a connector body adapted for connection with a tube,
opposing first and second latch arms resiliently joined to the connector body at flexing points, and
a first lever attached to the first latch arm and a second lever attached to the second latch arm, the first and second levers extending alongside the first and second latch arms, respectively, such that pressing the first and second levers towards each other deflects the first and second latch arms relative to the connector body about the flexing points.

2. The tube connector of claim 1, wherein the connector body comprises a tube connection point and wherein the first latch arm and the first lever are arranged on a first side of the tube connection point, and the second latch arm and the second lever are arranged on a second side of the tube connector point, opposite to the first side.

3. The tube connector of claim 2, wherein the first latch arm is disposed between the first lever and the tube connection point, and wherein the second latch arm is disposed between the second lever and the tube connection point.

4. The tube connector of claim 3, wherein the connector body is substantially planar in a plane, and wherein the first and second latch arms and the first and second levers are arranged in substantially the same plane as the plane of the connector body.

5. The tube connector of claim 4, wherein the tube connection point is substantially level with the plane of the connector body.

6. The tube connector of any of claims 2 to 5, wherein the first and second latch arms, first and second levers, and the tube connection point are aligned in the plane of the connector body.

7. The tube connector of any of claims 1 to 6, wherein the first and second latch arms each comprise a latch.

8. The tube connector of claim 7, wherein the latch of the first latch arm extends towards the first lever, and wherein the latch of the second latch arm extends towards the second lever.

9. The tube connector of any of claims 1 to 8, wherein the first lever is joined to the first latch arm along a portion of the first latch arm, and wherein the second lever is joined to the second latch arm along a portion of the second latch arm.

10. The tube connector of claim 9, wherein the first and second levers extend further from the connector body than the first and second latch arms, respectively.

11. The tube connector of any of claims 1 to 10, wherein the first and second levers each comprise a gripping surface.

12. The tube connector of any of claims 1 to 11, further comprising at least one guide arm extending from the connector body.

13. The tube connector of claim 12, wherein the at least one guide arm is substantially parallel with the first and second latch arms.

14. The tube connector of claim 12 or 13, wherein the at least one guide arm is arranged in substantially the same plane as the first and second latch arms.

15. The tube connector of any of claims 1 to 14, wherein the tube connector comprises a front portion having a fluid connector for fluidly coupling to an infusion part of the infusion set, and a rear portion comprising a tube connection for the tube, and wherein the flexing points are arranged at the rear portion.

16. The tube connector of claim 15, wherein the first and second latch arms and the first and second levers extend from the rear portion towards the front portion.

17. The tube connector of claim 16, wherein the first and second latch arms and the first and second levers extend beyond the front portion of the tube connector.

18. An infusion set comprising an infusion portion having a cannula and a mounting portion for mounting the infusion portion to a patient's skin with the cannula in a subcutaneous position, and a tube connector according to any one of claims 1 to 17.

19. The infusion set of claim 18, comprising at least one opening for the first and/or second latch arm, the opening comprising a latch portion for engagement by the first and/or second latch arm.

20. The infusion set of claim 19, comprising a first opening for the first latch arm and a second opening for the second latch arm, each of the first and second openings comprising a latch portion for engagement by the first and second latch arms, respectively.

21. The infusion set of any of claims 18 to 20, wherein the infusion portion further comprises a guide opening for a guide arm of the tube connector.

22. The infusion set of any of claims 18 to 21, wherein the infusion portion comprises a body portion, and wherein when the tube connector is connected to the infusion part, the first and second levers are arranged alongside the body portion and spaced therefrom.

23. An infusion system comprising the infusion set of any one of claims 18 to 22, and a source of medicament for delivery to the patient via the tube connector and the cannula.

24. The infusion system of claim 23, further comprising a pump for continuous or intermittent delivery of medicament to the patient.

25. An infusion set insertion device for attaching an infusion set to a patient, the infusion set insertion device comprising an insertion device body and a button operable to move an infusion set within the insertion device body, and wherein the button and/or the insertion device body comprises an opening adapted to receive a safety connector such that the safety connector prevents movement of the button relative to the insertion device body, and wherein the safety connector comprises:
a connector body,
opposing first and second latch arms resiliently joined to the connector body at flexing points, and
a first lever attached to the first latch arm and a second lever attached to the second latch arm, the first and second levers extending alongside the first and second latch arms, respectively, such that pressing the first and second levers towards each other deflects the first and second latch arms relative to the body portion about the flexing points.

26. An infusion portion of an infusion set, the infusion portion comprising:
a cannula for subcutaneous delivery of a medicament when the infusion portion is attached to a patient's skin, and
a spigot having a side wall with a regular polygonal cross-section having a number of sides exactly divisible by four, and
a plurality of fluid ports disposed in the side wall of the spigot, each of the plurality of fluid ports being connected with the cannula.

27. The infusion portion of claim 26, wherein the side wall has at least four sides, for example four sides, or eight sides, or twelve sides, or sixteen sides.

28. The infusion portion of claim 26 or claim 27, wherein each side of the side wall comprises one of the plurality of fluid ports.

29. The infusion portion of claim 28, wherein each side comprises a recess, and wherein on each side the fluid port is located in the recess.

30. The infusion portion of any of claims 26 to 29, wherein the spigot further comprises a flange edge overhanging the side wall.

31. The infusion portion of claim 30, wherein the flange edge comprises a plurality of guides.

32. The infusion portion of claim 30, wherein each of the plurality of guides comprises a recess aligned with one of the plurality of fluid ports.

33. The infusion portion of any of claims 26 to 32, further comprising a base portion, wherein the spigot protrudes from the base portion.

34. The infusion portion of any of claims 26 to 33, wherein the base portion comprises a plurality of guides.

35. The infusion portion of claim 34, wherein each of the plurality of guides comprises a recess aligned with one of the plurality of fluid ports.

36. The infusion portion of any of claims 26 to 35, further comprising a cannular chamber in fluid communication with the cannular, and wherein each of the plurality of fluid ports is in communication with the cannular chamber.

37. The infusion portion of any of claims 26 to 36, wherein each of the plurality of fluid ports comprises a seal, for example a septum seal.

38. A tube connector for connecting to an infusion portion having a side wall with a regular polygonal cross section with a number of sides exactly divisible by four, and a plurality of fluid ports, the tube connector comprising:
a first arm and a second arm spaced from and parallel to the first arm, and
a needle extending between the first and second arms,
wherein the first arm is spaced from the second arm such that the first and second arms engage opposite sides of the side wall of the infusion portion when the tube connector is connected to the infusion portion to align the needle with a fluid port between the opposite sides.

39. The tube connector of claim 38, further comprising a guide arranged to engage a corresponding guide of the infusion portion when the tube connector is connected to the infusion portion.

40. The tube connector of claim 39, wherein the guide comprises a protrusion arranged to engage a recess of the infusion portion when the tube connector is connected to the infusion portion.

41. The tube connector of any of claims 38 to 40, further comprising a connecting tube and a fluid path connecting the connecting tube to the needle.

42. The tube connector of any of claims 38 to 41, further comprising a rotatably mounted locking member, and wherein when the tube connector is connected to the infusion portion the locking member is rotatable to engage the infusion portion and hold the tube connector to the infusion portion.

43. The tube connector of claim 42, wherein the locking member comprises a loop member, and wherein when the tube connector is connected to the infusion portion the loop member is rotatable to at least partly surround a part of the infusion portion.

44. The tube connector of claim 42 or claim 43, wherein the locking member is rotatably mounted to the tube connector about a rotational axis perpendicular to the needle.

45. The tube connector of any of claims 42 to 44, wherein the locking member comprises a catch configured to retain the locking member in the locked position.

46. The tube connector of any of claims 38 to 45, wherein the first arm comprises a first latch and the second arm comprises a second latch, and wherein the first latch and the second latch are arranged to clip onto the spigot of the infusion portion when the tube connector is connected to the infusion portion.

47. The tube connector of claim 46, wherein the first latch and the second latch are arranged to engage recesses on opposite sides of the spigot when the tube connector is connected to the infusion portion.

48. The tube connector of claim 46, comprising a first loop member and a second loop member joined together via an opening, wherein the second loop member is sized such that the infusion portion can protrude through the second loop member, and wherein the first loop member comprises the first arm, the second arm, and the needle.

49. An infusion set comprising the infusion portion of any one of claim 26 to claim 37 and the tube connector of any one of claim 38 to claim 48.

50. An infusion system comprising the infusion set of claim 49 and a source of medicament, for a medicament pump such as an insulin pump.

51. A tube connector for an infusion set, the tube connector comprising:
a body portion comprising a tube connection point for connection with a tube and a fluid connector for fluidly coupling to an infusion portion of the infusion set,
a first latch arm and a second latch arm extending from the body portion on opposite sides of the fluid connector, and
a first tab attached to the first latch arm and a second tab attached to the second latch arm,
wherein the first and second tabs are attached to the first and second latch arms, respectively, on sides opposite to the fluid connector and extend over the body portion and spaced from the body portion such that pressing the first tab and the second tab towards the body portion deflects the first and second latch arms, respectively.

52. The tube connector of claim 51, wherein the first latch arm and the second latch are joined to the body portion at respective first and second flexing points.

53. The tube connector of claim 52, wherein the first and second flexing points are adapted to flex when the first tab and the second tab are pressed towards the body portion.

54. The tube connector of claim 52 or 53, wherein the body portion comprises a front side and a rear side, the tube connection point is disposed on the rear side, the fluid connector is disposed on the front side, and the first and second flexing points are arranged on the rear side.

55. The tube connector of any one of claims 51 to 54, wherein the first latch arm comprises a first latch and the second latch arm comprises a second latch, and wherein the first and second latches extend from the first and second latch arms in a direction towards the first and second tabs.

56. The tube connector of any one of claims 51 to 55, wherein the first tab and the second tab are spaced apart from each other proximate to a central part of the body portion.

57. The tube connector of claim 56, wherein the fluid connector is disposed in the central part of the body portion.

58. The tube connector of any one of claims 51 to 57, wherein the fluid connector comprises a needle.

59. The tube connector of claim 58, wherein the first latch arm and the second latch arm are parallel to the needle.

510. The tube connector of claim 58 or 59, wherein the first latch arm and the second latch arm are arranged in the same plane as the needle.

61. The tube connector of any one of claims 51 to 60, wherein the first latch arm and the second latch arm protrude beyond a front side of the body portion.

62. The tube connector of any one of claims 51 to 61, further comprising at least one guide arm extending from the body portion.

63. The tube connector of claim 62, wherein the at least one guide arm extends parallel to the first latch arm and the second latch arm.

64. The tube connector of claim 62 or 63, wherein the at least one guide arm is disposed between of the first and second latch arms.

65. The tube connector of any one of claims 62 to 64, wherein the at least one guide arm is in the same plane as the first and second latch arms.

66. The tube connector of any one of claims 62 to 65, wherein the at least one latch arm does not deflect when the first and second tabs are pressed towards the body portion.

67. An infusion set comprising an infusion portion having a cannula and a mounting portion for mounting the infusion portion to a patient's skin with the cannula in a subcutaneous position, and a tube connector according to any one of claims 51 to 66.

68. The infusion set of claim 67, comprising at least one opening for the first and/or second latch arm, the opening comprising a latch portion for engagement by the first and/or second latch arm.

69. The infusion set of claim 68, comprising a first opening for the first latch arm and a second opening for the second latch arm, each of the first and second openings comprising a latch portion for engagement by the first and second latch arms, respectively.

70. The infusion set of claim 67, wherein the infusion portion further comprises a guide opening for at least one guide arm of the tube connector.

71. An infusion system comprising the infusion set of any one of claims 67 to 70, and a source of medicament for delivery to the patient via the tube connector and the cannula.

72. The infusion system of claim 71, further comprising a pump for continuous or intermittent delivery of medicament to the patient.

73. An infusion set insertion device for attaching an infusion set to a patient, the infusion set insertion device comprising an insertion device body and a button operable to move an infusion set within the insertion device body, and wherein the button and/or the insertion device body comprises an opening adapted to receive a safety connector such that the safety connector prevents movement of the button relative to the insertion device body, and wherein the safety connector comprises:
a body portion having a central part,
a first latch arm and a second latch arm extending from the body portion on opposite sides of the central part, and
a first tab attached to the first latch arm and a second tab attached to the second latch arm,
wherein the first and second tabs are attached to the first and second latch arms, respectively, on sides opposite to the central part and extend over the body portion and spaced from the body portion such that pressing the first tab and the second tab towards the body portion deflects the first and second latch arms, respectively.

74. A tube connector for an infusion set, the tube connector comprising:
a connector body having a tube connection point for connection with a tube and a fluid path for connecting the tube with the infusion set during use,
an arm arranged to engage the infusion set when the tube connector is connected to the infusion set, and
a loop portion extending from the connector body such that a user can pull the loop portion to disconnect the tube connector from the infusion set.

75. The tube connector of claim 74, comprising a first arm and a second arm, the second arm being parallel to and spaced from the first arm.

76. The tube connector of claim 75, wherein the first arm and the second arm each comprise a latch for connecting the tube connector to the infusion set.

77. The tube connector of any one of claims 74 to 76, further comprising a needle extending from the connector body and connected to the fluid path, the needle being arranged to engage a fluid port of the infusion set when the tube connector is connected to the infusion set.

78. The tube connector of claim 77, wherein the needle is parallel with the latch arm.

79. The tube connector of any one of claims 74 to 78, wherein the loop portion is rotatably connected to the connector body.

80. The tube connector of claim 79, wherein the arm extends from the connector body in an axial direction, and wherein the loop portion is rotatably connected to the connector body about a rotational axis perpendicular to the axial direction.

81. The tube connector of claim 79 or 80, wherein, when the tube connector is connected to the infusion set, the loop portion is rotatable to engage the infusion set to retain the tube connector on the infusion set.

82. The tube connector of claim 81, wherein the loop portion is configured to clip over a part of the infusion set.

83. The tube connector of claim 81 or 82, wherein the infusion set comprises a spigot having a side wall with a fluid port.

84. The tube connector of claim 81 or 82, wherein the side wall of the infusion set comprises a plurality of fluid ports, and wherein the tube connector is configured to be connected to the infusion set at any one of the plurality of fluid ports.

85. An infusion set comprising an infusion portion having a cannula and a mounting portion for mounting the infusion portion to a patient's skin with the cannula in a subcutaneous position, and a tube connector according to any one of claim 74 to claim 84.

86. The infusion set of claim 85, wherein one of the tube connector and the infusion portion comprises a needle.

87. An infusion system comprising the infusion set of any one of claim 85 to claim 86, and a source of medicament for delivery to the patient via the tube connector and the cannula.

88. The infusion system of claim 87, further comprising a pump for continuous or intermittent delivery of medicament to the patient.

89. An infusion set insertion device for attaching an infusion set to a patient, the infusion set insertion device comprising an insertion device body and a button operable to move an infusion set within the insertion device body, and wherein the button and/or the insertion device body comprises a connector opening adapted to receive a safety connector such that the safety connector prevents movement of the button relative to the insertion device body, and wherein the safety connector comprises:
an arm for engaging the infusion set, and
a loop portion extending from the connector body such that a user can pull the loop portion to disconnect the tube connector from the infusion set.

90. A tube connector for an infusion set, the tube connector comprising:
a connector body having a tube connection point for connection with a tube and a fluid path for connecting the tube with the infusion set during use,
an arm arranged to engage the infusion set when the tube connector is connected to the infusion set, and
a locking member rotatably attached to the connector body and being rotatable to engage the infusion portion when the tube connector is connected to the infusion portion to retain the tube connector on the infusion set.

91. The tube connector of claim 90, wherein the locking member is shaped to at least partly surround a part of the infusion set when in the locked position.

92. The tube connector of claim 90 or 91, wherein the locking member comprises a loop portion.

93. A tube connector for connecting to an infusion portion of an infusion set, the tube connector comprising:
a first loop portion and a second loop portion that together define a closed loop,
wherein the first loop portion is larger than the second loop portion and sized such that the first loop portion can be positioned over a spigot of the infusion portion,
wherein the second loop portion is sized to receive the spigot when the tube connector is moved sideways and the spigot of the infusion portion moves from the first loop portion into the second loop portion, and
wherein the tube connector further comprises a fluid connection disposed in the second loop portion for forming a fluid connection between the tube connector and the infusion portion.

94. The tube connector of claim 93, wherein the second loop portion comprises a resiliently deflectable arm arranged to engage the spigot of the infusion portion.

95. The tube connector of claim 94, wherein the second loop portion comprises a first resiliently deflectable arm and an opposing second resiliently deflectable arm, the first and second resiliently deflectable arms being arranged to engage opposite sides of the spigot of the infusion portion.

96. The tube connector of claim 94 or 95, wherein the or each resiliently deflectable arm comprises a latch arranged to latch onto the spigot of the infusion portion.

97. The tube connector of any one of claims 93 to 96, wherein the fluid connection of the tube connector comprises a needle protruding into the second loop portion.

98. The tube connector of any one of claims 93 to 97, wherein the spigot of the infusion portion comprises a polygonal cross-section having opposing parallel sides, and wherein the tube connector further comprises an opening between the first loop portion and the second loop portion, the opening being sized such that the spigot can only pass through the opening when the direction of movement is parallel to the opposing parallel sides of the spigot.

99. The tube connector of claim 98, further comprising at least one protrusion extending into the closed loop, and wherein the opening is defined between the at least one protrusion and an opposite side of the tube connector.

100. The tube connector of any one of claims 93 to 99, wherein the second loop portion is shaped to match a cross-sectional shape of the spigot of the infusion portion.

101. The tube connector of any one of claims 93 to 100, further comprising a connecting tube in fluid communication with the fluid connection.

102. The tube connector of any one of claims 93 to 101, wherein a part of the first loop portion opposite to the second loop portion is shaped to match a portion of the spigot.

103. An infusion portion for an infusion set, the infusion portion comprising a base portion for attachment to a patient's skin and a cannula for subcutaneously delivering a medicament during use, wherein the infusion portion comprises a spigot extending from the base portion, the spigot comprising a fluid port for fluid connection with the tube connector of any one of claim 93 to claim 102.

104. The infusion portion of claim 103, wherein the spigot comprises a polygonal cross-sectional shape having a plurality of sides.

105. The infusion portion of claim 104, wherein the spigot comprises a generally triangular cross-sectional shape with the fluid port arranged at one of the tips of the spigot, and wherein the second loop portion of the tube connector comprises a partial triangular profile to receive the spigot and align the fluid port and the fluid connection.

106. The infusion portion of claim 104, wherein the spigot comprises a regular polygonal cross-sectional shape, the fluid port being arranged on one side of the spigot, and wherein the second loop portion of the tube connector comprises a partial regular polygonal profile to receive the spigot and align the fluid port and the fluid connection.

107. The infusion portion of claim 106, wherein the spigot comprises a plurality of fluid ports disposed in different sides of the spigot.

108. The infusion portion of claim 106 or 107, wherein each side of the spigot comprises a recess.

109. The infusion portion of any one of claims 103 to 108, wherein the spigot comprises a flange edge overhanging a side wall of the spigot opposite to the base portion, and wherein when the spigot is received in the second loop portion of the tube connector the second loop portion is disposed between the base portion and the flange edge.

110. An infusion set comprising the tube connector of any one of claim 93 to claim 102 and the infusion portion of any one of claim 103 to claim 109.

111. An infusion system comprising the infusion set of claim 110 and a source of medicament, for example a medicament pump such as an insulin pump.
